SEQUENCE LISTING (51 sequences)

SEQ ID NO: 1 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 194 aa, Homo sapiens
MKWKALFTAAILQAQLPITASSLPHPTQQSPEKKVLGPGGCTCRHNRFCNEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 195 aa, Homo sapiens
MKWKALFTAAILQAQLPITASSLPHPTQQSPEKKVLGPGGCTCRHNRFCNEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 6 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 7 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 8 - protein, 127 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 9 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 10 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 11 - protein, 102 aa, Homo sapiens
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 12 - protein, 101 aa, Homo sapiens
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 13 - protein, 112 aa, Homo sapiens
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 14 - protein, 188 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 15 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 16 - protein, 125 aa, Homo sapiens
MARLALSPVPSHWMVALLLLLSAEPVPAARSEDRYRNPKGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 17 - protein, 126 aa, Homo sapiens
MARLALSPVPSHWMVALLLLLSAAEPVPAARSEDRYRNPKGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 18 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 19 - protein, 230 aa, Homo sapiens
MARLALSPVPSHWMVALLLLLSAAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 20 - protein, 164 aa, Homo sapiens; identical to SEQ ID NO: 1

SEQ ID NO: 21 - protein, 164 aa, Artificial Sequence
  Synthetic
MKWKALFTAAILQAQLPITEAQSFGLLDPKLCYLLDGILFIYGVILTALFLRVKFSRSADAPAYQQGQNQLFNELNLGRREEFDVLDKRRGRDPEMGGKPQRRKNPQEGLFNELQKDKMAEAFSEIGMKGERRRGKGHDGLFQGLSTATKDTFDALHMQALPPR

SEQ ID NO: 22 - protein, 164 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 23 - protein, 164 aa, Artificial Sequence
  Synthetic
MKWKVSVLACILHVRFPGAEAQSFGLLDPKLCYLLDGILFIYGVIITALYLRAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPR

SEQ ID NO: 24 - protein, 164 aa, Artificial Sequence
  Synthetic
MKWKALFTAAILQAQLPITEAQSFGLLDPKLCYLLDGILFIYGVILTALFLRVKFSRSADAPAYQQGQNQLANELNLGRREEADVLDKRRGRDPEMGGKPQRRKNPQEGLANELQKDKMAEAASEIGMKGERRRGKGHDGLAQGLSTATKDTADALHMQALPPR

SEQ ID NO: 25 - protein, 164 aa, Artificial Sequence
  Synthetic
MKWKVSVLACILHVRFPGAEAQSFGLLDPKLCYLLDGILFIYGVIITALYLRAKFSRSAETAANLQDPNQLANELNLGRREEADVLEKKRARDPEMGGKQQRRRNPQEGVANALQKDKMAEAASEIGTKGERRRGKGHDGLAQGLSTATKDTADALHMQTLAPR

SEQ ID NO: 26 - protein, 8 aa, Artificial Sequence
  Synthetic
SIINFEKL

SEQ ID NO: 27 - protein, 8 aa, Artificial Sequence
  Synthetic
SAINFEKL

SEQ ID NO: 28 - protein, 8 aa, Artificial Sequence
  Synthetic
SIYNFEKL

SEQ ID NO: 29 - protein, 8 aa, Artificial Sequence
  Synthetic
SIIQFEKL

SEQ ID NO: 30 - protein, 8 aa, Artificial Sequence
  Synthetic
SIITFEKL

SEQ ID NO: 31 - protein, 8 aa, Artificial Sequence
  Synthetic
SIIVFEKL

SEQ ID NO: 32 - protein, 8 aa, Artificial Sequence
  Synthetic
SIIGFEKL

SEQ ID NO: 33 - protein, 8 aa, Artificial Sequence
  Synthetic
EIINFEKL

SEQ ID NO: 34 - protein, 71 aa, Mus musculus
MKWKVSVLACILHVRFPGAEAQSFGLLDPKLCYLLDGILFIYGVIITALYLRAKFSRSAETAANLQDPNQL

SEQ ID NO: 35 - protein, 51 aa, Mus musculus
MKWKVSVLACILHVRFPGAEAQSFGLLDPKLCYLLDGILFIYGVIITALYL

SEQ ID NO: 36 - protein, 41 aa, Mus musculus
NSRRNRLLQSDYMNMTPRRPGLTRKPYQPYAPARDFAAYRP

SEQ ID NO: 37 - protein, 92 aa, Artificial Sequence
  Synthetic
MKWKVSVLACILHVRFPGAEAQSFGLLDPKLCYLLDGILFIYGVIITALYLNSRRNRLLQSDYMNMTPRRPGLTRKPYQPYAPARDFAAYRP

SEQ ID NO: 38 - protein, 48 aa, Mus musculus
SVLKWIRKKFPHIFKQPFKKTTGAAQEEDACSCRCPQEEEGGGGGYEL

SEQ ID NO: 39 - protein, 99 aa, Artificial Sequence
  Synthetic
MKWKVSVLACILHVRFPGAEAQSFGLLDPKLCYLLDGILFIYGVIITALYLSVLKWIRKKFPHIFKQPFKKTTGAAQEEDACSCRCPQEEEGGGGGYEL

SEQ ID NO: 40 - protein, 24 aa, Mus musculus
YRRTRPHRSYTGPKTVQLELTDHA

SEQ ID NO: 41 - protein, 75 aa, Artificial Sequence
  Synthetic
MKWKVSVLACILHVRFPGAEAQSFGLLDPKLCYLLDGILFIYGVIITALYLYRRTRPHRSYTGPKTVQLELTDHA

SEQ ID NO: 42 - protein, 36 aa, Mus musculus
RKAWRLPNTPKPCWGNSFRTPIQEEHTDAHFTLAKI

SEQ ID NO: 43 - protein, 87 aa, Artificial Sequence
  Synthetic
MKWKVSVLACILHVRFPGAEAQSFGLLDPKLCYLLDGILFIYGVIITALYLRKAWRLPNTPKPCWGNSFRTPIQEEHTDAHFTLAKI

SEQ ID NO: 44 - protein, 15 aa, Mus musculus
YQPLKDREYDQYSHL

SEQ ID NO: 45 - protein, 66 aa, Artificial Sequence
  Synthetic
MKWKVSVLACILHVRFPGAEAQSFGLLDPKLCYLLDGILFIYGVIITALYLYQPLKDREYDQYSHL

SEQ ID NO: 46 - protein, 15 aa, Mus musculus
YQPLRDREDTQYSRL

SEQ ID NO: 47 - protein, 66 aa, Artificial Sequence
  Synthetic
MKWKVSVLACILHVRFPGAEAQSFGLLDPKLCYLLDGILFIYGVIITALYLYQPLRDREDTQYSRL

SEQ ID NO: 48 - protein, 15 aa, Mus musculus
YEPIRKGQRDLYSGL

SEQ ID NO: 49 - protein, 66 aa, Artificial Sequence
  Synthetic
MKWKVSVLACILHVRFPGAEAQSFGLLDPKLCYLLDGILFIYGVIITALYLYEPIRKGQRDLYSGL

SEQ ID NO: 50 - protein, 55 aa, Mus musculus
KNRKAKAKPVTRGTGAGSRPRGQNKERPPPVPNPDYEPIRKGQRDLYSGLNQRAV

SEQ ID NO: 51 - protein, 106 aa, Artificial Sequence
  Synthetic
MKWKVSVLACILHVRFPGAEAQSFGLLDPKLCYLLDGILFIYGVIITALYLKNRKAKAKPVTRGTGAGSRPRGQNKERPPPVPNPDYEPIRKGQRDLYSGLNQRAV

(19) United States
(12) Patent Application Publication
(10) Pub. No.: US 2024/0018210 A1
(43) Pub. Date: Jan. 18, 2024

(54) ENHANCED ANTIGEN REACTIVITY OF IMMUNE CELLS EXPRESSING A MUTANT NON-SIGNALING CD3 ZETA CHAIN

(71) Applicant: THE UNITED STATES OF AMERICA, AS REPRESENTED BY THE SECRETARY, DEPARTMENT OF HEALTH AND HUMAN SERVIC, Bethesda, MD (US)

(72) Inventors: Paul E. Love, Olney, MD (US); Guillaume Gaud, Bethesda, MD (US); Christian S. Hinrichs, Princeton, NJ (US); John S. Davies, Bethesda, MD (US)

(21) Appl. No.: 18/036,112

(22) PCT Filed: Nov. 12, 2021
(86) PCT No.: PCT/US2021/059109

§ 371 Date: May 9, 2023

Related U.S. Application Data
(60) Provisional application No. 63/113,428, filed on Nov. 13, 2020.

(51) Int. Cl. C07K 14/725; A61K 39/00

(52) U.S. Cl. CPC C07K 14/7051; A61K 39/4611; A61K 39/4632; A61K 39/4631; A61K 2239/22

(57) ABSTRACT

Disclosed is a cell expressing a modified CD3 subunit chain or a cell expressing a modified non-CD3 subunit chain comprising one or more of: (a) at least one Immuno-receptor Tyrosine-based Activation Motif (ITAM) deletion; or (b) at least one exogenous intracellular hematopoietic cell signaling domain; and (c) at least one modified ITAM comprising an amino acid sequence of Formula I. Related populations of cells, pharmaceutical compositions, methods of making the cells, methods of treating or preventing a condition in a subject, and methods of enhancing an antigen-specific immune response in a subject are also disclosed.

### CROSS-REFERENCE TO RELATED APPLICATION

This patent application claims the benefit of U.S. Provisional Patent Application No. 63/113,428, filed Nov. 13, 2020, which is incorporated by reference in its entirety herein.

### STATEMENT REGARDING FEDERALLY SPONSORED RESEARCH OR DEVELOPMENT

This invention was made with Government support under project number 1ZIAHD001803-23 by the National Institutes of Health, National Institute of Child Health and Human Development and project number 1ZIABC011479 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 48,349 Byte ASCII (Text) file named “757735_ST25.TXT,” dated Nov. 9, 2021.

### BACKGROUND OF THE INVENTION

Immunotherapy can be an effective treatment for a variety of conditions in some patients. However, obstacles to the overall success of immunotherapy still exist. For example, reactivity against a target antigen can be attenuated. Despite considerable research in the field of immunotherapy, there still exists a need for improved methods and products for immunotherapy.

### BRIEF SUMMARY OF THE INVENTION

An aspect of the invention provides a cell expressing a modified CD3 subunit chain comprising one or more of:
(a) at least one Immuno-receptor Tyrosine-based Activation Motif (ITAM) deletion;
(b) at least one exogenous intracellular hematopoietic cell signaling domain; and
(c) at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈ and X₉, is, independently, any amino acid residue;
each one of X₅ is, independently, any amino acid residue;
m is 6, 7, 8, 9, 10, 11, or 12; and
   wherein the cell expresses an antigen-specific receptor, wherein the antigen is a cancer antigen, autoimmune disease self-antigen, or infectious disease antigen,
   wherein the cell is not an immortalized cell line, and
   wherein the modified CD3 subunit chain is not comprised in a chimeric antigen receptor (CAR).

Another aspect of the invention provides a T cell receptor (TCR) negative cell expressing one or more of:
(a) a modified CD3 subunit chain or non-CD3 subunit chain comprising at least one ITAM deletion;
(b) a modified CD3 subunit chain or non-CD3 subunit chain comprising at least one exogenous intracellular hematopoietic cell signaling domain; and
(c) a modified CD3 subunit chain or a non-CD3 modified ITAM containing subunit chain comprising at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈ and X₉ is, independently, any amino acid residue;
each one of X₅ is, independently, any amino acid residue; and
m is 6, 7, 8, 9, 10, 11, or 12.

Another aspect of the invention provides a method of treating or preventing a condition in a subject, the method comprising administering a cell, or a population thereof, to the subject, in an amount effective to treat or prevent the condition in the subject, wherein the cell expresses one or more of:
(a) a modified CD3 subunit chain or non-CD3 subunit chain comprising at least one ITAM deletion;
(b) a modified CD3 subunit chain or non-CD3 subunit chain comprising at least one exogenous intracellular hematopoietic cell signaling domain; and
(c) a modified CD3 subunit chain or non-CD3 modified ITAM containing subunit chain comprising at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈, and X₉ is, independently, any amino acid residue;
each one of X₅ is, independently, any amino acid residue;
m is 6, 7, 8, 9, 10, 11, or 12; and
wherein the modified CD3 subunit chain is not comprised in a CAR.

Still another aspect of the invention provides a method of enhancing an antigen-specific immune response in a subject, the method comprising administering a cell, or a population thereof, to the subject, in an amount effective to enhance the antigen-specific immune response in the subject, wherein the cell expresses one or more of:
(a) a modified CD3 subunit chain or non-CD3 subunit chain comprising at least one ITAM deletion;
(b) a modified CD3 subunit chain or non-CD3 subunit chain comprising at least one exogenous intracellular hematopoietic cell signaling domain; and
(c) a modified CD3 subunit chain or non-CD3 modified ITAM containing subunit chain comprising at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈ and X₉ is, independently, any amino acid residue;
each one of X₅ is, independently, any amino acid residue;
m is 6, 7, 8, 9, 10, 11, or 12; and
   wherein the modified CD3 subunit chain is not comprised in a CAR.

Additional aspects of the invention provide populations of cells comprising at least one inventive cell, pharmaceutical compositions comprising the inventive cells or populations of cells, and methods of making the inventive cells.

### BRIEF DESCRIPTION OF THE SEVERAL VIEWS OF THE DRAWING(S)

FIG. 1A is a schematic illustrating an outline of the targeting strategy for making a mouse model expressing 6F CD3zeta. The schematic illustrates the TCR/CD3zeta locus, the 6Y/6F targeting construct, the 6Y knock-in allele generated by homologous recombination in embryonic stem (ES) cells, and the 6F knock-in allele generated after Cre-mediated removal of the 6Y-FLAG-NEO cassette.

FIG. 1B is a schematic of the TCR complexes expressed in the 6Y CD3zeta (6Y/6Y) mouse and the 6F CD3zeta (6F/6F) mouse, according to aspects of the invention.

FIGS. 2A-2B are graphs showing the tumor area (mm²) measured in C57BL/6 mice at the indicated number of days following treatment with OT-1 TCR transgenic CD8⁺ T cells expressing TCRs containing 6Y CD3zeta (“Ctrl;” diamonds) or 6F CD3zeta (triangles). The OT-1 TCR transgene encodes TCRalpha and TCRbeta chains that together confer recognition of OVA (or OVA-variant) peptides. Expression of the transgene results in expression of OVA-reactive TCRs on all T cells. Tumor cells expressed the high affinity ovalbumin peptide N4 (FIG. 2A) or the low affinity ovalbumin altered peptide V4 (FIG. 2B). Untreated tumor-bearing mice (circles) served as a control.

FIGS. 2C-2D are graphs showing the tumor area (mm²) measured in T/B cell deficient Rag1^{-/-} mice at the indicated number of days following treatment with OT-1 TCR transgenic CD8⁺ T cells expressing TCRs containing 6Y CD3zeta (“Ctrl;” diamonds) or 6F CD3zeta (triangles). Tumors expressed the high affinity ovalbumin peptide N4 (FIG. 2C) or the low affinity ovalbumin altered peptide V4 (FIG. 2D). Untreated tumor-bearing mice (circles) served as a control.

FIGS. 3A-3B are graphs showing T-cell mediated cytolysis (cell index) of a B16F10 melanoma tumor cell line at the indicated number of hours following co-culture with the indicated numbers of CD8⁺ T cells from OT-1 TCR transgenic 6Y/6Y mice (Ctrl), solid lines, or OT-1 TCR transgenic 6F/6F (6F, dotted lines) mice. The tumor cell line expressed the high affinity ovalbumin peptide N4 (FIG. 3A) or the low affinity ovalbumin altered peptide V4 (FIG. 3B). TRITON x-100 surfactant was used as a positive control for lysis. Tumor cells cultured alone (no effector T cells) served as a negative control.

FIG. 4 is a photographic image of an SDS-PAGE gel showing the results of a study of the signaling intensity of 6F CD3zeta TCRs compared to 6Y CD3zeta TCRs. CD8⁺ T cells from OT-1 TCR transgenic 6Y/6Y CD3zeta mice (CRE−; OT-1 6Y/6Y) and ERT2CRE+OT-1 TCR transgenic 6Y/6Y mice (CRP; OT-1 6Y/6Y) were treated with tamoxifen in vitro to induce 6Y to 6F switch in the CRE+ but not in the CRE⁻ cells (efficient switching was verified by intracellular staining for the Flag (6Y) or Myc (6F) epitope-tagged CD3zeta proteins; not shown). T cells were then stimulated with MHC-1 tetramers that contained the indicated peptides (OVA, T4, G4) or left unstimulated (NS). Cells were lysed and run on SDS-PAGE gels then blotted onto nitrocellulose membranes. Membranes were blotted with the indicated blotting antibody. 4G10 (pan pTyr) recognizes all tyrosine phosphorylated proteins. Actin blot was done as a loading control.

FIGS. 5A-5F are graphs showing the results of an experiment testing the TCR-mediated activation of acutely 6Y-6F switched OT-1 TCR transgenic CD8⁺ T cells. CD8⁺ T cells were purified from ERT2-Cre; OT-1 TCR transgenic 6Y/6Y (Cre⁺) and ERT2-Cre⁻; OT-1 TCR transgenic 6Y/6Y (Cre⁻) mice then treated with tamoxifen to induce switch of CD3zeta from 6Y to 6F in the ERT2Cre⁺ (circles) but not in the ERT2Cre⁻ cells (squares). T cells were then stimulated with antigen presenting cells pulsed with high affinity (OVA) (FIGS. 5A-5B), medium affinity (Q4) (FIGS. 5E-5F) or low affinity (T4) (FIGS. 5C-5D) peptides at the concentrations (M) shown. T cells were analyzed for expression of the activation markers CD25 (FIGS. 5A, 5C, and 5E) or CD69 (FIGS. 5B, 5D, and 5F) by flow cytometry (mean fluorescence intensity (MFI)) after 24 hrs.

FIG. 6 is a heat map showing IL-2 production by 6Y to 6F switched OT-1 TCR transgenic CD8⁺ T cells and non-switched ERT2Cre⁻ cells after stimulation with antigen presenting cells pulsed with the ovalbumin peptide (N4) or the weaker partial agonist peptides (in order: Q4, Q7, V4 or G4) at the concentrations and for the times shown. IL-2 was measured at the indicated times. A=genotype. B=peptide. C=concentration. Lighter shaded boxes indicate higher IL-2 concentration.

FIGS. 7A-7B are graphs (from the results depicted in FIG. 6) showing the concentration of IL-2 produced by 6Y to 6F switched OT-1 TCR transgenic CD8⁺ T cells (Cre⁺ (6F/6F)) (squares) and non-switched ERT2Cre⁻ cells (Cre− (6Y/6Y)) (circles) after stimulation with antigen presenting cells pulsed with the ovalbumin peptide (N4) (FIG. 7A) or the weaker partial agonist peptide V4 (FIG. 7B) at the concentrations (nM) and for the times (hours) shown.

FIGS. 8A-8C are graphs showing the proliferation of naïve CD8 T cells stimulated with increasing amounts of anti-CD3 (ng/ml) alone (8A and 8B) or in combination with anti-CD28 (1 mg/ml) (8A and 8C) assessed by Cell Trace Violet (CTV) dilution (8A). Graphs (8B-8C) show combined data from three separate experiments. Data were analyzed by unpaired t-test (two tailed) and are represented as mean±SEM. *P<0.05, **P<0.01.

FIGS. 9A-9D are graphs showing the percentage of CD5 (9A and 9B) and CD69 (9C and 9D) activation marker expressed by peripheral naive CD8 T cells from mice of the indicated genotype stimulated with increasing amounts of anti-CD3 (ng/ml) alone (9A and 9C) or in combination with anti-CD28 (1 μg/ml) (9B and 9D). Statistical comparisons are 6Y/6Y to 6Y/6Y Ert2Cre (+T) (top) or 6F/6F (bottom). Data were analyzed by unpaired t-test (two tailed) and are represented as mean±SEM. *P<0.05, **P<0.01.

FIG. 10 shows an image of a gel showing the activation of signaling effectors in naive CD8 T cells of the indicated genotype stimulated with 10 μg/ml of anti-CD3 antibody.

FIGS. 11A-11E are graphs showing the results of experiments in which mice of the indicated genotype were infected with LCMV Armstrong and spleen T-cells were analyzed for cytokine expression 8 days later after re-stimulation with peptide NP396 (11A), NP205 (11B), GP276 (11C), GP33 (11D), or GP61 (11E). N=4. Data were analyzed by unpaired t-test (two tailed) and are represented as mean±SEM. *P<0.05, **P<0.01, ***P<0.001. Results shown are representative of at least three experiments.

FIG. 12A shows a heatmap of IL-2 production by OTI CD8 T cells of the indicated genotype co-cultured with APC pulsed with the indicated peptides.

FIGS. 12B-12C show graphs of the time-course of IL-2 production following stimulation with N4 (12B) or G4 (12C) peptides. Statistical significance determined by unpaired t-test (two-tailed) analysis. ***P<0.001, ns not significant.

FIGS. 13A-13D are graphs showing IL-2 (13A), IFN-gamma (13B), IL-6 (13C), and TNF-alpha (13D) concentrations in supernatant of OTI-stimulated cell cultures as detailed for FIGS. 12A-12C. NP: No peptide. Statistical significance determined by unpaired t-test (two-tailed) analysis. *P<0.05, **P<0.01, ***P<0.001, ns not significant.

FIGS. 14A-14E are graphs showing in vivo proliferation of CTV-loaded Thy1.2 CD8 T cells co-injected into Thy1.1/1.2 hosts at 1:1 ratio followed by injection of N4 (14A and 14C), Q4 (14A and 14D), T4 (14A and 14E), peptide-pulsed APCs (or APCs pulsed with no peptide (14A and 14B)) 24 h later. Spleen and lymph node T cells were analyzed 6 days after injection of APCs. Data shown are representative of two experiments.

FIGS. 15A-15C and 16 are graphs showing IL-6 (15A), IFN-gamma (15B), and TNFalpha (15C) response or CD69 expression (FIG. 16) of OTI CD8 T cells from mice of the indicated genotype stimulated with APC pulsed with b-catenin self-peptide (Catnb) for 72 h or 24 h, respectively. ns not significant.

FIG. 17 is a graph showing the results of an experiment in which 6Y/6Y or 6Y/6Y Ert2-cre (Thy1.2) naïve T cells were treated in vitro with 4-OH tamoxifen [6Y/6Y Ert2-cre (+T)] and then co-injected with Thy1.1 (WT) naive T cells at 1:1 ratio into Rag2^{-/-} mice. Thy1.2/Thy1.1 ratio of T cells from lymph nodes was analyzed 6 days after injection. Data representative of three experiments.

FIGS. 18A-18B are graphs showing the results of an experiment in which bone marrow chimeras were generated with a 1:1 mix of WT (CD45.1) and either 6Y/6Y Ert2Cre (CD45.2) or +/+Ert2Cre (CD45.2) T-depleted bone marrow cells. Mice received tamoxifen by oral gavage once daily for 5 days and were then sacrificed 2 weeks after the last gavage. Graphs show the percentage of CD4+(18A) and CD8+(18B) naïve and memory CD45. 2 T cells from lymph nodes. Results shown are representative of two experiments.

FIGS. 19A-19G, 20A-20G, 21A-21G, and 22A-22G show heatmaps (19A, 20A, 21A, 22A) and graphs (19B-19G, 20B-20G, 21B-21G, and 22B-22G) showing the results of experiments in which OTI CD8 T Cells from 6Y/6Y and 6Y/6Y dLck-Cre mice were co-cultured with APC pulsed with 1 mM of the indicated peptide N4 (19B, 20B, 21B, 22B), Q4 (19C, 20C, 21C, 22C), T4 (19D, 20D, 21D, 22D), V4 (19E, 20E, 21E, 22E), G4 (19F, 20F, 21F, 22F), or E1 (19G, 20G, 21G, 22G) and expression of IL-2 (19A-19G), IFNγ (20A-20G), IL-6 (21A-21G) or TNFα (22A-22G) was assessed in supernatant at the indicated times. Graphs of cytokine measurements are shown as Log 10(cytokine/LOD). Statistical significance determined by two-tailed ANOVA test analysis 1. *P<0.05, **P<0.01, ***P<0.001, ****P<0.0001, ns, not significant.

FIGS. 23A-23G, 24A-24G, 25A-25G, and 26A-26G show heatmaps (23A, 24A, 26A) and graphs (23B-23G, 24B-24G, 25B-25G, and 26B-26G) showing the results of experiments in which OTI CD8 T cells of the indicated genotypes were treated in vitro with 4-OH tamoxifen before experiments. OTI CD8 T cells from 6Y/6Y and 6Y/6Y Ert2-Cre (+T) mice were co-cultured with APC pulsed with 10-9 M of the indicated peptide N4 (23B, 24B, 26B), Q4 (23C, 24C, 25C, 26C), T4 (23D, 24D, 25D, 26D), V4 (23E, 24E, 25E, 26E), or G4 (23F, 24F, 25F, 26F) and expression of IL-2 (23A-23F), IFNγ (24A-24F), IL-6 (25A- or TNFα (26A-26F) was assessed in supernatant at the indicated times. Graphs of cytokine measurements are shown as Log 10(cytokine/LOD). Statistical significance determined by two-tailed ANOVA test analysis. *P<0.05, **P<0.01, ns, not significant.

FIGS. 27A-27D are graphs showing the mean of logarithm of concentrations over time normalized by the lower limit of detection (LOD) of IL-2 (27A), IFNγ (27B), TNFα (27C) and IL-6 (27D) in supernatant of 6Y/6Y and 6Y/6Y Ert2-Cre (+T) OTI-stimulated cells. Statistical significance determined by two-tailed ANOVA multiple comparison test analysis. *P<0.05, **P<0.01, ***P<0.001 ns. Graph shows mean±SEM of three independent experiments.

FIGS. 28 and 29A-29D present graphs showing the results of experiments in which OTI 6Y/6Y and OTI 6Y/6Y Ert2-Cre (+T) CD8 T cells (FIG. 28) or OTI 6Y/6Y and OTI 6Y/6Y dLck-Cre CD8 T cells (FIGS. 29A-29D) were stimulated with APC pulsed with the indicated concentration of peptides and analyzed for cytokine production in the supernatant (FIG. 28) and CD69 surface expression (FIGS. 29A (N4), 29B (Q4), 29C (T4), and 29D (G4)). Statistical significance determined by unpaired t-test analysis (FIG. 28) or two-tailed ANOVA test analysis (FIGS. 29A-29D). *P<0.05, **P<0.01, ***P<0.001, ****P<0.0001, ns not significant. Data representative of three experiments.

FIG. 30 is a graph showing the results of an experiment in which OTI 6Y/6Y or OTI 6Y/6Y Ert2-Cre (+T) CD8 T cells were stimulated with peptide-pulsed APCs for 48 h. Graph shows the MFI of IRF4 expression from intracellular staining. Representative of two experiments. Statistical significance determined by unpaired t-test analysis **P<0.01, ns, not significant.

FIG. 31 is a graph showing the results of an experiment in which OTI CD8 T cells from mice of the indicated genotype were treated in vitro with 4-OH tamoxifen and stimulated for 2 minutes with peptide-pulsed APCs. Graph shows the percentage of pLAT (Y171) positive cells from intracellular staining. Representative of two experiments.

FIG. 32 shows an image of gel showing the results of an experiment in which OTI CD8 T cells from mice of the indicated genotype were stimulated with K^{b} peptide-tetramers and analyzed by PAGE and immunoblot. Representative of three experiments.

FIGS. 33-36 show images of gels showing the results of experiments in which OTI 6Y/6Y and OTI 6F/6F (FIG. 33) or OTI 6Y/6Y dLck-Cre (FIGS. 34-36) CD8 T cells were left unstimulated or stimulated for 2 minutes with the indicated K^{b} peptide-tetramers N4, Q4, and T4 (FIG. 34), Q4 and T4 (FIG. 35), N4 and G4 (FIG. 36) and analyzed by immunoblot after immunoprecipitation with the indicated antibody and PAGE. Representative of three experiments. IP-immunoprecipitated, CL-cell lysate.

FIGS. 37A-37C are graphs showing the results of experiments in which OTI CD8 T cells from mice of the indicated genotype were stimulated for 2 minutes with peptide-pulsed APCs. Graph represents the percentage of pZAP-70 (Y319) (37A), pLAT (Y171) (37B) or pErk1/2 (T202/Y204) (37C) positive cells from intracellular staining. Representative of two experiments. Statistical significance determined by unpaired t-test analysis *P<0.05, **P<0.01, ***P<0.001, ns, not significant.

FIG. 38 shows an image of gel resulting from an experiment in which 6Y/6Y and 6Y/6Y Ert2-Cre OTI CD8 T cells were treated in vitro with 4-OH tamoxifen [6Y/6Y Ert2-Cre (+T)] and stimulated with the indicated k^{b} peptide-tetramers and analyzed by PAGE and immunoblot. Representative of three experiments.

FIGS. 39-40 show images of gels resulting from experiments in which OTI 6Y/6Y and 6F/6F (FIG. 40) or OTI 6Y/6Y dLck-Cre (FIG. 39) CD8 T cells were stimulated with the indicated k^{b-}tetramers and analyzed by immunoblot with the indicated antibodies after immunoprecipitation and PAGE. Representative of 2 (FIG. 40) or three (FIG. 39) experiments, respectively. IP-immunoprecipitated, CL-cell lysate.

FIGS. 41-42 show the results of a FACS analysis showing CD62L vs CD44 surface staining (FIG. 41), or CD5 and TCRb surface staining (Mean Fluorescence Intensity; MFI) of OTI CD8 T cells of the indicated genotypes after in vitro activation and expansion (FIG. 42).

FIGS. 43A-43B are graphs showing the results of experiments in which expanded OTI CTL were stimulated with APC pulsed with the indicated concentration of peptides and analyzed for CD25 surface expression (A) or IFNγ expression (B) by FACS. Bar graph plots show MFI of CD25 (A) or % IFNγ⁺ cells (B). Statistical significance determined by unpaired t-test analysis. *P<0.05, **P<0.01, ***P<0.001, ****P<0.0001, ns, not significant. Data are representative of at least two experiments.

FIGS. 44A-44H are graphs showing the results in vitro tumor killing assays of OTI CD8 T cells from mice of the indicated genotypes against B16F10-N4 or B16F10-APL expressing target cells at various effector-to-target ratios. FIGS. 44A (N4), 44C (Q4), 44E (T4), and 44G (V4) are curves showing the cell index plotted against time after adding T cells. Arrow represents the time of OTI CTL addition to the melanoma cell cultures. FIGS. 44B (N4), 44D (Q4), 44F (T4), and 44H (V4) are graphs showing the related killing index. Data are represented as mean±S.E.M. Statistical significance determined by ratio paired t-test analysis 1. *P<0.05, **P<0.01, ***P<0.001, ****P<0.0001, ns, not significant.

FIGS. 45A-45B are graphs showing the measurement of the size of B16F10-N4 (A) or -V4 (B) tumors implanted into C57BL/6 mice that were injected with OTI CD8 T cells 7 days after B16F10 melanoma implantation. Data are represented as mean±S.E.M. Statistical significance determined by ratio paired t-test analysis 1. *P<0.05, **P<0.01, ***P<0.001, ****P<0.0001, ns, not significant.

FIG. 45C-45D are survival plots from the experiment of FIGS. 45A-45B with respect to B16F10-N4 (C) or -V4 (D) tumors. Data are represented as mean±S.E.M.

FIG. 46 is a graph showing the measurement of the size of B16F10-V4 tumors implanted into T/B cell deficient Rag1^{-/-} mice that were injected with OTI CD8 T cells 7 days after B16F10 melanoma implantation. Data are represented as mean±S.E.M.

FIG. 47 is a survival plot from experiment shown in FIG. 46. Data are represented as mean±S.E.M.

FIGS. 48A-48B are graphs showing the results of experiments in which OTI CD8 T cells of the indicated genotypes were treated in vitro with 4-OH tamoxifen for 48 h and activated with 0.5 mM N4 peptide for 6 days before experiments. Shown are in vitro killing assay of OTI CD8 T cells from the indicated mice against B16F10-N4 (A) or B16F10-V4 (B) target cells at the indicated effector-to-target ratios.

FIG. 49 shows an image of a gel showing the results of an experiment in which WT (+/+) OTI CD8 CTL were transduced with retroviruses expressing 2A-epitope tagged 6Y-CD3z (6Y) or 6F-CD3z (6F) before addition to tumor cell cultures. Transduced OTI CTLs were analyzed by immunoblot with the indicated antibodies after immunoprecipitation with anti-TCRb antibody and PAGE. IP-immunoprecipitated, CL-cell lysate.

FIGS. 50A-50B are graphs showing the results of in vitro B16F10-N4 (A) or B16F10-V4 (B) tumor killing assays of transduced OTI CTLs of indicated genotypes at 10:1 or 2.5:1 effector-to-target ratios. UT, untransduced.

FIG. 51 is a graph showing the results of an experiment in which OTI CD8 T cells of the indicated genotype were activated in vitro with 0.5 mM N4 peptide for 6 days before experiment. Graph shows an in vitro killing assay experiment of OTI CD8 T cells against B16F10-N4 target cells at various effector-to-target ratios. Data are representative of three experiments and are represented as mean±S.E.M.

FIGS. 52A-52D are graphs showing the results of experiments in which OTI CD8 T cells of the indicated genotype were activated in vitro with 0.5 mM N4 peptide for 6 days before experiment. FIGS. 52A-52B show tumor growth curves of C57BL/6J mice injected with B16F10-N4 (A) or -V4 (B) expressing tumors that received OTI CD8 T cells from mice of the indicated genotype 7 days after B16F10 melanoma cell implantation. FIGS. 52C-52D show survival curves corresponding to FIGS. 52A-52B, respectively. Data are representative of three experiments and are represented as mean±S.E.M. Statistical significance determined by ratio paired t-test analysis. *P<0.05.

FIGS. 53A-53C are schematics. Without being bound to a particular theory or mechanism, these schematics represent models depicting the effect of 6F-CD3z on TCR signaling when initiated by low or high affinity TCR-pMHC interactions. FIG. 53A shows that unstimulated ‘resting’ peripheral T cells receive homeostatic self-pMHC TCR interactions that result in partial phosphorylation of CD3z ITAMs. Mono-phosphorylation of CD3 ITAM(s) in 6Y-CD3z (wild-type) TCRs and 6F-CD3z TCRs leads to recruitment of the tyrosine phosphatase SHP1. FIG. 53B shows that engagement of 6Y-CD3z TCRs by high affinity ligands with a long TCR/pMHC dwell time results in dual phosphorylation of most TCR ITAMs and strong signaling. Some ITAMs are mono-phosphorylated resulting in recruitment of SHP1. Engagement of 6F-CD3z TCRs by high affinity ligands with a long TCR/pMHC dwell time results in dual phosphorylation of all 4 (CD3e,g,d) ITAMs and moderate/strong signaling. SHP1 is not recruited. FIG. 53C shows that engagement of 6Y-CD3z TCRs by low affinity ligands with a short pMHC dwell time results in mono-phosphorylation of some CD3z ITAMs and recruitment of SHP1, which results in a weak/inhibited signal. Engagement of 6F-CD3z TCRs by low affinity ligands with a short pMHC dwell time results in dual phosphorylation of all 4 (CD3e,g,d) ITAMs. Mono-phosphorylation of CD3z ITAMs does not occur in 6F-CD3z TCRs and SHP1 is not recruited to the TCR, resulting in the generation of a moderate signal. P indicates phosphorylated ITAM. Black bar indicates ITAM where the tyrosines are mutated to phenylalanine.

FIG. 54A is a schematic of a TCR complex comprising a modified CD3zeta chain comprising a truncated intracellular domain lacking any intracellular T-cell signaling domains, according to an aspect of the invention.

FIG. 54B is a schematic of a TCR complex comprising a modified CD3zeta chain in which the 6 tyrosines (Y) within the ITAMs are mutated to alanine (A), according to an aspect of the invention.

FIGS. 55A-55G are schematics of a TCR complex comprising a modified CD3zeta chain comprising a CD28 (A), Icos (B), 4-1BB (C), OX40 (D), CD40 (E), CD40L (F), or CD27 (G) intracellular T-cell signaling domain and a deletion of all 3 ITAMs, according to an aspect of the invention.

FIG. 55H is a schematic of a TCR complex comprising a modified CD3zeta chain comprising a CD27 intracellular T-cell signaling domain, a CD28 intracellular T-cell signaling domain, and a deletion of all 3 ITAMs, according to an aspect of the invention.

FIGS. 56A-56C are schematics of a TCR complex comprising a modified CD3zeta chain comprising an intracellular T-cell signaling domain of a CD3gamma chain (A), CD3delta chain (B), or CD3epsilon chain (C) and a deletion of all 3 ITAMs, according to an aspect of the invention.

FIGS. 57A-57B are graphs showing T-cell mediated cytolysis (cell index) of a B16F10 melanoma tumor cell line at the indicated number of hours following co-culture with the indicated CD8⁺ T cells from OT-1 TCR transgenic mice. The CD8⁺ OT-1 T cells were transduced with a vector expressing a CD3zeta chain with all of the ITAM tyrosines (Y) (6Y), a CD3zeta chain where all three ITAMs were deleted (truncated), a CD3zeta chain in which all of the ITAM tyrosines (Y) were mutated to alanine (6A), or a CD3zeta chain in which all of the ITAM tyrosines (Y) were mutated to phenylalanine (6F). The tumor cell line expressed the high affinity ovalbumin peptide N4 (FIG. 57A) or the low affinity ovalbumin altered peptide V4 (FIG. 57B).

FIGS. 58A-58B are graphs showing T-cell mediated cytolysis (cell index) of a B16F10 melanoma tumor cell line at the indicated number of hours following co-culture with the indicated CD8⁺ T cells from OT-1 TCR transgenic mice. The CD8⁺ OT-1 T cells were transduced with a vector expressing a CD3zeta chain with all of the ITAM tyrosines (Y) (6Y), a CD3zeta chain where all three ITAMs were deleted and replaced by an intracellular T-cell signaling domain of 41BB, a CD3zeta chain where all three ITAMs were deleted and replaced by an intracellular T-cell signaling domain of ICOS, or a CD3zeta chain in which all of the ITAM tyrosines (Y) were mutated to phenylalanine (6F). The tumor cell line expressed the high affinity ovalbumin peptide N4 (FIG. 58A) or the low affinity ovalbumin altered peptide V4 (FIG. 58B).

FIGS. 59A-59B are graphs showing T-cell mediated cytolysis (cell index) of a B16F10 melanoma tumor cell line at the indicated number of hours following co-culture with the indicated CD8⁺ T cells from OT-1 TCR transgenic mice. The CD8⁺ OT-1 T cells were were transduced with a vector expressing a CD3zeta chain with all of the ITAM tyrosines (Y) (6Y), a CD3zeta chain where all three ITAMs were deleted and replaced by an intracellular T-cell signaling domain of CD28, a CD3zeta chain where all three ITAMs were deleted and replaced by an intracellular T-cell signaling domain of OX40, or a CD3zeta chain in which all of the ITAM tyrosines (Y) were mutated to phenylalanine (6F). The tumor cell line expressed the high affinity ovalbumin peptide N4 (FIG. 59A) or the low affinity ovalbumin altered peptide V4 (FIG. 59B).

### DETAILED DESCRIPTION OF THE INVENTION

The T cell Receptor (TCR) is the main signaling structure used by T cells to recognize and respond to target antigen, e.g., foreign antigen and tumor (neo) antigen. The TCR contains two subunits (TCRalpha and TCRbeta) that confer antigen binding and recognition. The TCRalpha and TCRbeta subunits assemble with six other (CD3) subunits that confer signal transducing capability on the TCR. The CD3 subunits assemble as dimers: a CD3gamma/CD3epsilon dimer, a CD3delta/CD3epsilon dimer, and a CD3zeta/CD3zeta dimer. A complete TCR complex is composed of eight subunits: a TCRalpha/TCRbeta dimer, a CD3gamma/CD3epsilon dimer, a CD3delta/CD3epsilon dimer, and a CD3zeta/CD3zeta dimer.

Each of the CD3 subunits (as well as some other non-CD3 signal transducing subunits) contain(s) one or more copies of a semi-conserved sequence designated the Immuno-receptor Tyrosine-based Activation Motif (ITAM). The wild-type (unsubstituted) consensus sequence for ITAMs is provided by Formula II:

YX₁X₂L/I—(X₃)ₚ-YX₄X₅L/I (Formula II)

wherein p is 6, 7, 8, 9, 10, 11, or 12; each of X₁, X₂, X₄, and X₅ is, independently, any amino acid residue; and each one of X₃ is, independently, any amino acid residue,
Y=Tyrosine, L=Leucine, I=Isoleucine. L/I indicates that the amino acid can be either Isoleucine or Leucine.
   See Reth, Nature, 338: 383-384 (1989). Although p is most commonly 6, 7, or 8 in Formula II, longer wild-type (unsubstituted) ITAM consensus sequences also exist, wherein p can be as high as 12 in Formula II. The CD3gamma, CD3delta and CD3epsilon subunits each contain a single ITAM, whereas CD3zeta contains three tandem ITAMs. Thus, the TCR complex contains a total of ten (10) ITAMs.

The tyrosine residues in each ITAM are involved in TCR signal transduction. Antigen binding by the TCR results in phosphorylation of the ITAM tyrosine residues by the Src kinase, Lck. The two tyrosine residues in each ITAM are appropriately spaced to bind (once phosphorylated) to the two SH2 domains in the T cell specific tyrosine kinase, ZAP-70. Binding of ZAP-70 to phosphorylated ITAM tyrosine residues results in activation of ZAP-70. Activation of ZAP-70 leads to activation of downstream signaling pathways that result in proliferation, cytokine production and, in the case of CD8⁺ T cells, cytolytic activity. ITAM phosphorylation is believed to be essential for TCR signaling activity. Mutation of ITAM tyrosines (Y) to phenylalanine (F), which cannot be phosphorylated, results in the complete loss of ITAM signaling function. Without being bound to a particular theory or mechanism, the prevailing view has been that multiple ITAMs act together to amplify TCR signals.

It has been discovered that a cell expressing a CD3zeta (CD3z or CD3ζ) subunit chain comprising modified ITAMs in which both of the native tyrosine residues have been substituted as described herein provides enhanced target antigen reactivity as compared to a cell expressing the CD3zeta subunit chain comprising only wild-type (unsubstituted) ITAMs. It has also been discovered that a CD3zeta subunit chain comprising three ITAM deletions as described herein provides enhanced target antigen reactivity as compared to a cell expressing the wild-type, unmodified CD3zeta subunit chain. It has also been discovered that a CD3zeta subunit chain comprising three ITAM deletions and at least one exogenous (non-ITAM) intracellular T-cell signaling domain as described herein provides enhanced target antigen reactivity as compared to a cell expressing the wild-type, unmodified CD3zeta subunit chain. These discoveries are contrary to the prevailing view of ITAM function in TCR signaling and are, therefore, surprising.

In an aspect of the invention, the inventive modified CD3 subunit chains or non-CD3 subunit chains described herein may enhance reactivity against a “low affinity” target antigen. Affinity is the strength with which one molecule (e.g., a receptor) binds to its ligand (e.g. an antigen) (Campillo-Davo et al., Cells, 9(7):1720 (2020)). Affinity may be measured, for example, by SPR Biacore assays, which provide a method for determining the binding kinetics of a ligand for its receptor. The technique measures the real-time binding association and dissociation rates using Surface Plasmon Resonance (SPR). Binding is measured as a change in resonance units (RUs) on the biosensor surface to which the receptor is attached. Affinity may be assessed by the association (kₒₙ) and dissociation (k_{off}) kinetics of the receptor-ligand complex. The affinities of TCRs against non-mutated cancer antigens (e.g., self-cancer peptides) may be lower than TCR affinities for foreign antigens (Stone et al., Curr. Opin. Immunol., 33: 16-22 (2015)). In an aspect of the invention, the inventive modified CD3 subunit chains or non-CD3 subunit chains described herein may enhance reactivity against non-mutated cancer antigens (e.g., self-cancer peptides) or cancer neo-antigens, wherein the non-mutated cancer antigens (e.g., self-cancer peptides) and cancer neo-antigens have a lower TCR affinity as compared to a foreign antigen. Cancer neo-antigens are derived from self-proteins but contain cancer specific mutations (usually single amino acid mutations) and usually differ from self only by the single amino acid difference. Accordingly, cancer neo-antigens also usually have a lower TCR affinity since they are comprised mostly of self amino acids. This differs from foreign antigens in which most or all of the amino acids are non-self configurations.

Although the above measures of affinity may be accurate, they may fail to translate well to biological systems, where a particular biological response may be of interest (biological responses may include, for example, expression of activation markers and cytokines and/or killing efficiency of T cells towards tumor target cells). Also, affinity alone may not account for ligand concentration and the contribution of other molecules such as co-receptors to the strength of a receptor-ligand interaction when these molecules are expressed on living cells. The term “avidity” includes both the affinity (strength of one receptor-ligand binding pair) plus ligand concentration (effect of having a few or many receptor-ligand interactions) and the contribution of co-receptors (Campillo-Davo et al., Cells, 9(7):1720 (2020)).

In an aspect of the invention, the inventive modified CD3 subunit chains or non-CD3 subunit chains described herein may enhance “functional avidity” against a target antigen, e.g., a “low affinity” target antigen. The term “functional avidity” describes cell-based receptor-ligand interactions (Campillo-Davo et al., Cells, 9(7):1720 (2020)). Functional avidity uses a biological response such as those described above and may record the response over a range of ligand concentrations. Functional avidity may be expressed as EC50 (Effective concentration-50), which is the ligand concentration that produces a half-maximal biological response. In the Examples, ligand affinity is used to describe the known Biacore binding strength peptides since this may be useful when comparing different ligands. However, the assays described in the Examples are based on functional avidity (how well the T cells are activated, how much they proliferate, and/or how much cytokine they produce or how well they kill tumor target cells) in response to different ligand concentrations to evaluate the T cell responses.

### Cells, Populations of Cells, and Pharmaceutical Compositions

An aspect of the invention provides a cell expressing a modified CD3 subunit chain comprising one or more of:
(a) at least one ITAM deletion;
(b) at least one exogenous intracellular hematopoietic cell signaling domain; and
(c) at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈ and X₉, is, independently, any amino acid residue;
each one of X₅ is, independently, any amino acid residue;
m is 6, 7, 8, 9, 10, 11, or 12; and
   wherein the cell expresses an antigen-specific receptor, wherein the antigen is a cancer antigen, autoimmune disease self-antigen, or infectious disease antigen,
   wherein the cell is not an immortalized cell line, and
   wherein the modified CD3 subunit chain is not comprised in a CAR.

In an aspect of the invention, the cell expresses a modified CD3 subunit chain comprising at least one ITAM deletion. In aspects of the invention, the modified CD3 subunit chain comprises at least two ITAM deletions or three ITAM deletions. In another aspect of the invention, all ITAMs are deleted from the modified CD3 subunit chain. As an example, the CD3 subunit chain may be truncated such that it contains at least one ITAM deletion, at least two ITAM deletions, three ITAM deletions, or no ITAMs at all. In an aspect of the invention, the modified CD3 subunit chain comprising at least one ITAM deletion has a truncated intracellular domain lacking any intracellular T-cell signaling domains, including the lack of any modified or unmodified ITAMS.

In an aspect of the invention, the cell expressing a modified CD3 subunit chain comprises at least one exogenous intracellular hematopoietic cell (e.g., T-cell) signaling domain. By “exogenous,” with respect to the intracellular hematopoietic cell (e.g., T-cell) signaling domain, is meant that the intracellular hematopoietic cell signaling domain is not native to (naturally-occurring on) the wild-type CD3 subunit chain corresponding to the modified CD3 subunit chain. The modified CD3 subunit chain may comprise at least one exogenous intracellular hematopoietic cell signaling domain in addition to, or instead of, one or both of the at least one ITAM deletion and/or the at least one modified ITAM, both as described herein with respect to other aspects of the invention. For example, the modified CD3 subunit chain may comprise at least one ITAM deletion and an exogenous intracellular hematopoietic cell signaling domain. For example, the modified CD3 subunit chain may comprise at least one exogenous intracellular hematopoietic cell signaling domain, none of the ITAM deletions described herein with respect to other aspects of the invention, none of the modified ITAMs described herein with respect to other aspects of the invention, and one or more endogenous, wild-type ITAMs. By “endogenous,” with respect to an ITAM, is meant that the ITAM is native to (naturally-occurring on) the wild-type CD3 subunit chain corresponding to the modified CD3 subunit chain. For example, the modified CD3 subunit chain may comprise no modifications as compared to the endogenous, wild-type CD3 subunit chain corresponding to the modified CD3 subunit chain with the exception that the modified CD3 subunit chain comprises at least one exogenous intracellular hematopoietic cell signaling domain.

The exogenous intracellular (i.e., cytoplasmic) hematopoietic cell (e.g., T-cell) signaling domain may be an intracellular T-cell signaling domain or other hematopoietic cell signaling domain that originates from any T-cell signaling protein or other hematopoietic cell protein known in the art other than the CD3 subunit chain being modified. In an aspect of the invention, exogenous intracellular hematopoietic cell signaling domain may be a non-ITAM-containing exogenous intracellular hematopoietic cell signaling domain. Examples of intracellular T-cell signaling domains may include, but are not limited to, the intracellular T-cell signaling domain of any one of the following proteins: a 4-1BB protein, a CD27 protein, a CD28 protein, a CD8-alpha protein, a CD40 protein, a CD40L protein, an Icos protein, an OX40 protein, or any combination of the foregoing.

In another aspect of the invention, the exogenous intracellular T-cell signaling domain may be an intracellular T-cell signaling domain that originates from any of the other CD3 subunit chains of the T cell receptor complex other than the CD3 subunit chain being modified. For example, the modified CD3 subunit chain may be:
(i) a CD3zeta chain comprising an ITAM deletion and an intracellular T-cell signaling domain of any one of the following proteins: a CD3gamma chain, CD3delta chain, CD3epsilon chain, or any combination of the foregoing;
(ii) a CD3gamma chain comprising an ITAM deletion and an intracellular T-cell signaling domain of any one of the following proteins: a CD3zeta chain, CD3delta chain, CD3epsilon chain, or any combination of the foregoing;
(iii) a CD3delta chain comprising an ITAM deletion and an intracellular T-cell signaling domain of any one of the following proteins: a CD3gamma chain, CD3zeta chain, CD3epsilon chain, or any combination of the foregoing;
(iv) a CD3epsilon chain comprising an ITAM deletion and an intracellular T-cell signaling domain of any one of the following proteins: a CD3gamma chain, CD3delta chain, CD3zeta chain, or any combination of the foregoing;
(v) a CD3zeta chain comprising an intracellular T-cell signaling domain of any one of the following proteins: a CD3gamma chain, CD3delta chain, CD3epsilon chain, or any combination of the foregoing;
(vi) a CD3gamma chain comprising an intracellular T-cell signaling domain of any one of the following proteins: a CD3zeta chain, CD3delta chain, CD3epsilon chain, or any combination of the foregoing;
(vii) a CD3delta chain comprising an intracellular T-cell signaling domain of any one of the following proteins: a CD3gamma chain, CD3zeta chain, CD3epsilon chain, or any combination of the foregoing; or
(viii) a CD3epsilon chain comprising an intracellular T-cell signaling domain of any one of the following proteins: a CD3gamma chain, CD3delta chain, CD3zeta chain, or any combination of the foregoing.

Non-limiting examples of amino acid sequences which may be included in modified CD3 subunit chains according to aspects of the invention are shown in Table 1. Shown in Table 1 are constructs using mouse sequences designed for proof of concept experiments in the mouse model. For experiments in human cells and for clinical applications, constructs will be designed that contain the corresponding human sequences to those shown for mouse. Not shown in Table 1 but also included in aspects of the invention are: Mouse CD3z and TM domain-Mouse CD3gamma cytoplasmic domain; Mouse CD3z and TM domain-Mouse CD3delta cytoplasmic domain; Mouse CD3z and TM domain-Mouse CD3gamma or CD3delta or CD3epsilon cytoplasmic domain plus one or more hematopoietic cell signaling domain(s); and human amino acid sequences analogous to each of the foregoing.

TABLE 1

| Description | Sequence |
|---|---|
| Mouse CD3z truncated in the intracellular | MKWKVSVLACILHVRFPGAEAQSFGL |
| domain at a position N-terminal to all | LDPKLCYLLDGILFIYGVIITALYLRAKF |
| three ITAMs | SRSAETAANLQDPNQL (SEQ ID NO: |
| | 34) |
| Mouse CD3z Extracellular (EC) and | MKWKVSVLACILHVRFPGAEAQSFGL |
| Transmembrane (TM) domain | LDPKLCYLLDGILFIYGVIITALYL (SEQ |
| | ID NO: 35) |
| Mouse CD28 intracellular (IC) domain | NSRRNRLLQSDYMNMTPRRPGLTRKP |
| | YQPYAPARDFAAYRP (SEQ ID NO: 36) |
| Mouse CD3z Extracellular (EC) and | MKWKVSVLACILHVRFPGAEAQSFGL |
| Transmembrane (TM) domain-Mouse CD28 | LDPKLCYLLDGILFIYGVIITALYLNSRR |
| IC domain | NRLLQSDYMNMTPRRPGLTRKPYQPY |
| | APARDFAAYRP (SEQ ID NO: 37) |
| Mouse 4-1BB IC domain | SVLKWIRKKFPHIFKQPFKKTTGAAQE |
| | EDACSCRCPQEEEGGGGGYEL (SEQ ID |
| | NO: 38) |
| Mouse CD3z EC and TM domain-Mouse4- | MKWKVSVLACILHVRFPGAEAQSFGL |
| 1BB IC domain | LDPKLCYLLDGILFIYGVIITALYLSVLK |
| | WIRKKFPHIFKQPFKKTTGAAQEEDAC |
| | SCRCPQEEEGGGGGYEL (SEQ ID NO: |
| | 39) |
| Mouse ICOS IC domain | YRRTRPHRSYTGPKTVQLELTDHA |
| | (SEQ ID NO: 40) |
| Mouse CD3z EC and TM domain-Mouse | MKWKVSVLACILHVRFPGAEAQSFGL |
| ICOS IC domain | LDPKLCYLLDGILFIYGVIITALYLYRRT |
| | RPHRSYTGPKTVQLELTDHA (SEQ ID |
| | NO: 41) |
| Mouse OX40 IC domain | RKAWRLPNTPKPCWGNSFRTPIQEEHT |
| | DAHFTLAKI (SEQ ID NO: 42) |
| Mouse CD3z EC and TM domain-Mouse | MKWKVSVLACILHVRFPGAEAQSFGL |
| OX40 IC domain | LDPKLCYLLDGILFIYGVIITALYLRKA |
| | WRLPNTPKPCWGNSFRTPIQEEHTDAH |
| | FTLAKI (SEQ ID NO: 43) |
| Mouse CD3gamma ITAM | YQPLKDREYDQYSHL (SEQ ID NO: 44) |
| Mouse CD3z Extracellular (EC) and | MKWKVSVLACILHVRFPGAEAQSFGL |
| Transmembrane (TM) domain-Mouse | LDPKLCYLLDGILFIYGVIITALYLYQPL |
| CD3 gamma ITAM | KDREYDQ YSHL (SEQ ID NO: 45) |
| Mouse CD3delta ITAM | YQPLRDREDTQYSRL (SEQ ID NO: 46) |
| Mouse CD3z EC and TM domain-Mouse | MKWKVSVLACILHVRFPGAEAQSFGL |
| CD3delta ITAM | LDPKLCYLLDGILFIYGVIITALYL |
| | YQPLRDREDTQYSRL (SEQ ID NO: 47) |
| Mouse CD3epsilon ITAM | YEPIRKGQRDLYSGL (SEQ ID NO: 48) |
| Mouse CD3z EC and TM domain-Mouse | MKWKVSVLACILHVRFPGAEAQSFGL |
| CD3epsilon ITAM | LDPKLCYLLDGILFIYGVIITALYLYEPI |
| | RKGQRDLYSGL (SEQ ID NO: 49) |
| Mouse CD3epsilon cytoplasmic domain | KNRKAKAKPVTRGTGAGSRPRGQNKE |
| | RPPPVPNPDYEPIRKGQRDLYSGLNQR |
| | AV (SEQ ID NO: 50) |
| Mouse CD3z EC and TM domain-Mouse | MKWKVSVLACILHVRFPGAEAQSFGL |
| CD3epsilon cytoplasmic domain | LDPKLCYLLDGILFIYGVIITALYLKNR |
| | KAKAKPVTRGTGAGSRPRGQNKERPP |
| | PVPNPDYEPIRKGQRDLYSGLNQRAV |
| | (SEQ ID NO: 51) |

In an aspect of the invention, the cell expressing a modified CD3 subunit chain comprises at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈ and X₉, is, independently, any amino acid residue;
each one of X₅ is, independently, any amino acid residue; and
m is 6, 7, 8, 9, 10, 11, or 12.

In an aspect of the invention, any one or more of X₁, X₂, X₃, X₄, X₅, X₆, X₇, X₈, and X₉ is, independently, a naturally occurring amino acid residue, with the proviso that at least one of X₁ and X₆ is not tyrosine. The naturally occurring amino acid residues include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In an aspect of the invention, at least one of X₁ and X₆ in Formula I is, independently, phenylalanine. In an aspect of the invention, at least one of X₁ and X₆ in Formula I is, independently, alanine. In an aspect of the invention, each of X₁ and X₆ in Formula I is phenylalanine. In an aspect of the invention, each of X₁ and X₆ in Formula I is alanine.

In an aspect of the invention, any one or more of X₁, X₂, X₃, X₄, X₅, X₆, X₇, X₈, and X₉ is, independently, an artificial (synthetic) amino acid residue. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

In an aspect of the invention, each of X₄ and X₉ is, independently, leucine or isoleucine.

In an aspect of the invention, the cell expressing the amino acid sequence of Formula I expresses an antigen-specific receptor (for example the T cell antigen receptor, TCR)). The phrases “antigen-specific” and “antigenic specificity,” as used herein, mean that the antigen-specific receptor can specifically bind to and immunologically recognize an antigen, or an epitope thereof, such that binding of the antigen-specific receptor to antigen, or the epitope thereof, elicits an immune response. The antigen-specific receptor may be exogenous or endogenous to the cell. By “exogenous,” with respect to the antigen-specific receptor, is meant that the antigen-specific receptor is not native to (naturally-occurring on) the cell. By “endogenous,” with respect to the antigen-specific receptor, is meant that the antigen-specific receptor is native to (naturally-occurring on) the cell.

Generally, the antigen-specific receptor may, but need not, comprise the modified CD3 subunit chain. In an aspect of the invention, the modified CD3 subunit chain is not comprised in, or associated with, the antigen-specific receptor expressed by the cell. For example, the cell may express both (1) a TCR complex that may or may not be antigen-specific with a CD3zeta chain comprising the modified ITAM amino acid sequence of Formula I and (2) an antigen-specific CAR which does not comprise the modified ITAM amino acid sequence of Formula I. In an aspect of the invention, the modified CD3 subunit chain is not comprised in a CAR.

In another aspect of the invention, the modified CD3 subunit chain is comprised in, or associated with, the antigen-specific receptor expressed by the cell. For example, the cell may express a TCR complex with a CD3zeta chain which comprises the modified ITAM amino acid sequence of Formula I.

In an aspect of the invention, the antigen-specific receptor is a TCR. For example, the antigen-specific receptor is an exogenous TCR. The exogenous TCR may be a recombinant TCR. A recombinant TCR is a TCR which has been generated through recombinant expression of one or more exogenous TCR α-, β-, γ-, and/or δ-chain encoding genes that assemble with endogenous CD3 chains. A recombinant TCR can comprise polypeptide chains derived entirely from a single mammalian species, or the recombinant TCR can be a chimeric or hybrid TCR comprised of amino acid sequences derived from TCRs from two different mammalian species. For example, the TCR can comprise a variable region derived from a human TCR, and a constant region of a murine TCR such that the TCR is “murinized.” Any exogenous TCR having antigenic specificity for a cancer antigen, autoimmune disease self-antigen, or infectious disease antigen may be useful in the inventive methods and compositions. The TCR generally comprises two polypeptides (i.e., polypeptide chains), such as an α-chain of an αβTCR, a β-chain of an αβTCR, a γ-chain of a γδTCR, a δ-chain of a γδTCR, or a combination thereof that are responsible for antigen recognition and that assemble with invariant CD3 signal transducing chains to form a complete TCR complex. Such polypeptide chains of TCRs are known in the art. The antigen-specific TCR can comprise any amino acid sequence, provided that the TCR can specifically bind to and immunologically recognize a cancer antigen (or epitope thereof), autoimmune disease self-antigen (or epitope thereof) or infectious disease antigen (or epitope thereof). Examples of exogenous TCRs that may be useful in the inventive methods and compositions include, but are not limited to, those disclosed in, for example, U.S. Pat. Nos. 7,820,174; 7,915,036; 8,088,379; 8,216,565; 8,431,690; 8,613,932; 8,785,601; 9,128,080; 9,345,748; 9,487,573; 9,822,162; 9,879,065; 10,174,098; U.S. Patent Application Publication Nos. 2013/0116167; 2014/0378389; and 2019/0135891, each of which is incorporated herein by reference. The exogenous TCR may be the anti-HPV 16 E7 TCR disclosed in U.S. Pat. No. 10,174,098, the anti-HPV 16 E6 TCR disclosed in U.S. Pat. No. 9,822,162, or the anti-KK-LC-1 TCR disclosed in U.S. Patent Application Publication No. 2019/0135891. In an aspect of the invention, the antigen specific exogenous TCR is synthetic, meaning that it is originally derived from a cloned natural TCR that is subsequently modified (mutated) to change the affinity with which it binds to its cognate antigen. For example, the cloned human TCR IG4 binds to an antigen termed NY-ESO-1 which is expressed by a large number of human transformed cell lines and solid tumors. Single amino acid substitution variant “affinity enhanced” IG4 TCRs, NY-ESO (95:LY) and NY-ESO (c259) have been generated that bind to the NY-ESO-1 peptide with different affinities than the wild-type IG4 TCR (Thomas et al., 9(947): 1-14 (2018)).

In an aspect of the invention, the antigen-specific receptor is a CAR. In an aspect of the invention, the CAR does not comprise the modified CD3 subunit chain. Typically, a CAR is a single chain receptor that comprises the antigen binding domain of an antibody, e.g., a single-chain variable fragment (scFv), fused to the transmembrane domain of a TCR subunit or a co-receptor and an intracellular domain of a TCR subunit (most often CD3zeta) that includes one or more ITAMs and frequently additional hematopoietic cell signaling motifs such as those from 4-1BB or CD28. Thus, the antigenic specificity of a CAR can be encoded by a scFv which specifically binds to the cancer antigen (or epitope thereof) or infectious disease antigen (or epitope thereof). Any CAR having antigenic specificity for a cancer antigen, autoimmune disease self-antigen or infectious disease antigen may be useful in the inventive methods and compositions. Examples of CARs that may be useful in the inventive methods and compositions include, but are not limited to, those disclosed in, for example, U.S. Pat. Nos. 8,465,743; 9,266,960; 9,765,342; 9,359,447; 9,868,774 and each of which is incorporated herein by reference.

In an aspect of the invention, the antigen-specific receptor is an endogenous TCR (for example, in the case of Tumor-Infiltrating-Lymphocytes (TILs)). In some aspects, the T cell comprising the endogenous TCR does not comprise (e.g., express) a CAR or an exogenous TCR. In other aspects, a T cell comprising an endogenous antigen-specific TCR can also be transformed, e.g., transduced or transfected, with one or more nucleic acids encoding an exogenous (e.g., recombinant) TCR or other recombinant receptor (e.g., CAR). Such exogenous receptors, e.g., TCRs, can confer specificity for additional antigens to the transformed T cell beyond the antigens for which the endogenous TCR is naturally specific. This can, but need not, result in the production of T cells having dual antigen specificities.

In an aspect of the invention, the antigen-specific receptor is a T cell receptor fusion construct (TRuC™) (TCR 2 Therapeutics, Cambridge, MA) (also referred to as T cell receptor fusion proteins). T cell receptor fusion constructs are disclosed in WO 2016/187349. T cell receptor fusion constructs comprise an antibody-based binding domain that is conjugated or fused to TCR subunit chains. Thus, the antigenic specificity of the T cell receptor fusion construct can be encoded by an scFv which specifically binds to the cancer antigen (or epitope thereof), autoimmune disease self-antigen (or epitope thereof) or infectious disease antigen (or epitope thereof). The T cell receptor fusion construct includes the endogenous TCR's six CD3 subunit chains. Unlike CARs, T cell receptor fusion constructs become a functional component of the endogenous TCR complex. Unlike TCRs, T cell receptor fusion constructs bind to antigen independent of the major histocompatibility complex (MHC).

In an aspect of the invention, the antigen-specific receptor has antigenic specificity for a cancer antigen. The term “cancer antigen,” as used herein, refers to any molecule (e.g., protein, polypeptide, peptide, lipid, carbohydrate, etc.) solely or predominantly expressed or over-expressed by a tumor cell or cancer cell, such that the antigen is associated with the tumor or cancer. The cancer antigen can additionally be expressed by normal, non-tumor, or non-cancerous cells. However, in such cases, the expression of the cancer antigen by normal, non-tumor, or non-cancerous cells is usually not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the cancer antigen can additionally be expressed by cells of a different state of development or maturation. For instance, the cancer antigen can be additionally expressed by cells of the embryonic or fetal stage, which cells are not normally found in an adult host. Alternatively, the cancer antigen can be additionally expressed by stem cells or precursor cells, which cells are not normally found in an adult host. Examples of cancer antigens include, but are not limited to, mesothelin, CD19, CD22, CD30, CD70, CD276 (B7H3), gp100, MART-1, Epidermal Growth Factor Receptor Variant III (EGFRVIII), Vascular Endothelial Growth Factor Receptor 2 (VEGFR-2), TRP-1, TRP-2, tyrosinase, human papillomavirus (HPV) 16 E6, HPV 16 E7, HPV 18 E6, HPV 18 E7, KK-LC-1, NY-BR-1, NY-ESO-1 (also known as CAG-3), SSX-2, SSX-3, SSX-4, SSX-5, SSX-9, SSX-10, MAGE-A1, MAGE-A2, BRCA, MAGE-A3, MAGE-A4, MAGE-A5, MAGE-A6, MAGE-A7, MAGE-A8, MAGE-A9, MAGE-A10, MAGE-A11, MAGE-A12, HER-2, etc. In an aspect of the invention, the cancer antigen may be a mutated antigen that is expressed or overexpressed by tumor or cancer cells and which is not expressed by normal, non-tumor, or non-cancerous cells. Examples of such cancer antigens may include, but are not limited to, mutated KRAS and mutated p53. T cells having antigenic specificity for a cancer antigen may, advantageously, reduce or avoid cross-reactivity with normal tissues such as, for example, that which may occur using T cells having antigenic specificity for minor histocompatability antigens. In a preferred aspect, the cancer antigen is HPV 16 E7, HPV 16 E6, HPV 18 E7, HPV 18 E6, or KK-LC-1. In an aspect of the invention, the cancer antigen is a non-mutated cancer antigen (e.g., self-cancer peptide) or cancer neo-antigen.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor, or non-cancer cells.

The term “infectious disease antigen” as used herein refers to any molecule (e.g., protein, peptide, lipid, carbohydrate, etc.) solely or predominantly expressed an agent that can cause an infection that can lead to a disease, such that the antigen is associated with the agent. Such agents may include, for example, bacteria, viruses, fungi, and parasites. Accordingly, the infectious disease antigen may be a bacterial antigen, viral antigen, fungal antigen, or parasite antigen.

Examples of viral antigens may include, but are not limited to, those expressed by herpes viruses, pox viruses, hepadnaviruses, papilloma viruses, adenoviruses, coronoviruses, orthomyxoviruses, paramyxoviruses, flaviviruses, and caliciviruses. Further examples of viral antigens may include, but are not limited to, respiratory syncytial virus (RSV), influenza virus, herpes simplex virus, Epstein-Barr virus, human immunodeficiency virus, varicella virus, cytomegalovirus, hepatitis A virus, hepatitis B virus, hepatitis C virus, human T-lymphotropic virus, calicivirus, adenovirus, and Arena virus. Viral antigens are known in the art and include, for example, any viral protein, e.g., env, gag, pol, gp120, thymidine kinase, and the like. In an aspect of the invention, the viral antigen may one associated with a cancer-causing virus such as, for example, HPV 16 E6, HPV 16 E7, HPV 18 E6, or HPV 18 E7.

Examples of bacterial antigens may include, but are not limited to, those expressed by a bacteria selected from the group consisting of Streptococcus pneumoniae, Neisseria Meningitides. Haemophilus influenzae, Streptococcus agalactiae, Listeria monocytogenes, Escherichia coli, Mycobacterium tuberculosis, Staphylococcus aureus, Pseudomonas aeruginosa, Ureaplasma urealyticum, Moraxeiia catarrhalis, Clostridium perfringens, Neisseria gonorrheae, Chlamydia trachomatis, Helicobacter pylori, Campylobacter jejuni, Salmonella enterica, Enterococcus faecalis, Clostridum difficile, Staphylococcus saprophytics, Treponema pallidum, Haemophilus ducreyi, Mycoplasma pneumoniae, Chlamydia pneumoniae, and Legionella pneumophila.

Examples of fungal antigens may include, but are not limited to, those expressed by Cryptococcus, Aspergillus, Coccidioides, Histoplasma, Blastomyces, and Pneumocystis.

The parasitic antigen may, for example, be an antigen expressed by any of the three main classes of parasites that can cause disease in humans: protozoa, helminths, and ectoparasites. Examples of parasitic antigens may include, but are not limited to, Sarcodina (e.g., Entamoeba), Mastigophora (e.g., Giardia, Leishmania), Ciliophora (Balantidium), Sporozoa (e.g., Plasmodium, Cryptosporidium), flatworms (platyhelminths) (e.g., trematodes (flukes) and cestodes (tapeworms), and roundworms (nematodes).

“Autoimmune disease self-antigen,” as used herein, refers to any protein, peptide, enzyme complex, ribonucleoprotein complex, and post-translationally modified antigen normally expressed by an individual and against which the individual's T cells are directed.

In an aspect of the invention, the cell expressing the modified CD3 subunit chain may be any cell which is capable of expressing a CD3 subunit chain and an antigen-specific receptor. In an aspect of the invention, the cell expressing the modified CD3 subunit chain is not an immortalized cell line. The cell may be a CD3 subunit positive cell. A CD3 subunit positive cell expresses any CD3 subunit chain such as, for example, a complete TCR receptor complex, or a portion thereof (specifically, one or more CD3 subunits).

In an aspect of the invention, the cell expressing the modified CD3 subunit chain is a T-cell (e.g., a TCR-expressing cell). For purposes herein, the T cell can be any T cell, e.g., a primary T cell or a T cell obtained from a mammal. If obtained from a mammal, the T cell can be obtained from numerous sources, including but not limited to blood, bone marrow, lymph node, the thymus, bone marrow, or other tissues or fluids. T cells can also be enriched for or purified. Preferably, the T cell is a human T cell. More preferably, the T cell is a T cell isolated from a human. In some cases, as in the case of TILs, antigen-specific T cells may be isolated, enriched for or purified from a tumor biopsy. The T cell can be any type of T cell and can be of any developmental stage, including but not limited to CD4⁺ helper T cell, e.g., Th₁ and Th₂ cell, CD4⁺ T cells, CD8⁺ T cell (e.g., cytotoxic T cell), tumor infiltrating lymphocyte (TIL), memory T cell (e.g., central memory T cell and effector memory T cell), naïve T cells, regulatory T cell (Treg), natural killer T (NKT) cell, mucosal-associated invariant T (MAIT) cell, gamma delta T cell (γδ T cells), or alpha beta (αβ) T cell, and the like. In another aspect of the invention, the cell is derived from an induced pluripotent stem cell (iPSC), embryonic stem cell or hematopoietic stem cell. In other aspect, the cell is derived from a hematopoietic stem cell.

It is contemplated that CD3 subunit chains may be useful for enhancing the immunological activity of other immune cells that express one or more CD3 subunits, but do not necessarily express a TCR. For example, cells which do not express a TCR but do express activating receptors that contain CD3zeta include natural killer (NK) cells and innate lymphoid cells (ILCs). These cell types may be involved in tumor killing and control of tumor growth (Marcus et al., Adv. Immunol., 122: 91-128 (2014); Ducimetiere et al., Front. Immunol., 10: 2895 (2019)). It is contemplated that modification of the native CD3 subunit chain expressed in such cells to comprise a modified CD3 subunit chain, e.g., with a modified ITAM amino acid sequence of Formula I, may improve signaling by any receptors that contain CD3 subunits (whether the cell expresses an antigen-specific receptor or not) and could improve the immunological activity of any CD3 subunit expressing cell.

In this regard, an aspect of the invention provides a TCR negative cell expressing a modified CD3 subunit chain or non-CD3 subunit chain comprising at least one ITAM deletion. The ITAM deletion may be as described herein with respect to other aspects of the invention. In an aspect of the invention, the modified CD3 subunit chain or non-CD3 modified ITAM containing subunit chain expressed by the TCR negative cell is not comprised in a CAR.

An aspect of the invention provides a TCR negative cell expressing a modified CD3 subunit chain or non-CD3 subunit chain comprising at least one exogenous intracellular T-cell or other hematopoietic cell signaling domain. The exogenous intracellular hematopoietic cell (e.g., T-cell) signaling domain may be as described herein with respect to other aspects of the invention.

An aspect of the invention provides a TCR negative cell expressing a modified CD3 subunit chain or a non-CD3 modified ITAM containing subunit chain comprising at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈, and X₉, is, independently, any amino acid residue;
each one of X₅ is, independently, any amino acid residue; and
m is 6, 7, 8, 9, 10, 11, or 12.

The amino acid residue of X₁, X₂, X₃, X₄, X₅, X₆, X₇, X₈, and X₉ is, independently, as described herein with respect to other aspects of the invention.

The TCR negative cell may be any TCR negative immune cell. For example, in an aspect of the invention, the TCR negative cell is an NK cell or an ILC. In another aspect of the invention, the TCR negative cell is derived from a hematopoietic stem cell. In another aspect of the invention, the cell is derived from an iPSC, embryonic stem cell or hematopoietic stem cell.

The modified CD3 subunit chain may be any CD3 subunit chain of the TCR complex (CD3zeta, CD3gamma, CD3delta or CD3epsilon). The CD3 subunit chain may be derived from any mammal, including any of the mammals described herein. For example, the CD3 subunit chain may be a mouse or a human CD3 subunit chain which has been modified as described herein with respect to other aspects of the invention. For example, the modified CD3 subunit chain can be a human CD3 subunit chain which has been modified to comprise the amino acid sequence of Formula I. Human CD3 subunit chain amino acid sequences are known in the art. Examples of human CD3 subunit chain amino acid sequences include, but are not limited to those set forth in Table 2.

TABLE 2

| Name | GenBank Accession No. | SEQ ID NO: |
|---|---|---|
| CD3zeta | NP_932170.1 | 1 |
| CD3zeta | NP_000725.1 | 2 |
| CD3zeta | NP_001365445.1 | 3 |
| CD3zeta | NP_001365444.1 | 4 |
| CD3epsilon | NP_000724.1 | 5 |
| CD3gamma | NP_000064.1 | 6 |
| CD3delta | NP_000723.1 | 7 |
| CD3delta | NP_001035741.1 | 8 |

In an aspect of the invention, the modified CD3 subunit chain is a CD3gamma chain, CD3delta chain, or CD3epsilon chain. In another aspect of the invention, the modified CD3 subunit chain is a CD3zeta chain. In an aspect of the invention, the cell may express only one modified CD3 subunit chain. In another aspect of the invention, the cell may express more than one modified CD3 subunit chain. For example, the cell may express any one or more of the following dimers, wherein only one member of the dimer is a modified CD3 subunit chain or wherein both members of the dimer comprise a modified CD3 subunit chain: a CD3gamma/CD3epsilon dimer, a CD3delta/CD3epsilon dimer, and a CD3zeta/CD3zeta dimer.

In an aspect of the invention, the CD3zeta chain comprises one, two or three modified ITAMs, wherein each modified ITAM comprises an amino acid sequence of Formula I. In another aspect of the invention, the CD3zeta chain comprises two or three modified ITAMs, wherein each modified ITAM comprises an amino acid sequence of Formula I.

In an aspect of the invention, the modified CD3 subunit chain is a subunit of non-TCR Immunoreceptors. Such receptors include but are not limited to: the Natural Cytotoxicity Receptors (NCRs) NKp30 and NKp46, Ly49, TREM receptors and FcgammaRIIIA.

In an aspect of the invention, the modified ITAM-containing subunit comprising at least one ITAM deletion, at least one exogenous intracellular hematopoietic cell signaling domain, or the amino acid sequence of Formula I is not a CD3 chain. Such molecules include but are not limited to: i) FcepsilonR1gamma which is a component of the Fc-epsilon RI receptor, the Fc-gamma RI receptor, the Fc-alpha R1 receptor and the Fc-gamma RIIIA receptor, ii) Tyrobp (DAP12) which is a component of NKp44, TREM1-3, KIR2, CD94, MDL1, SIRPbeta, CD200, CD300, PILRbeta, and SIGLEC14, and iii) Ig-alpha (CD79A) and Ig-beta (CD79B) which are components of the B cell antigen receptor. These non-CD3 subunits (e.g., non-CD3 ITAM containing subunits) are expressed by several immune cell types including B cells, NK cells, T cells, myeloid cells and hematopoietic cells and participate in signal transduction by the receptors they associate with. Examples of non-CD3 ITAM containing subunit amino acid sequences include, but are not limited to those set forth in Table 3.

TABLE 3

| Name | GenBank Accession No. | SEQ ID NO: |
|---|---|---|
| FcepsilonR1gamma: | NP_004097.1 | 9 |
| DAP12 (Tyrobp) | NP_003323.1 | 10 |
| DAP12 (Tyrobp) | NP_001166985.1 | 11 |
| DAP12 (Tyrobp) | NP_001166986.1 | 12 |
| DAP12 (Tyrobp) | NP_937758.1 | 13 |
| CD79A | EAW57075.1 | 14 |
| CD79A | EAW57076.1 | 15 |
| CD79B | EAW94227.1 | 16 |
| CD79B | EAW94228.1 | 17 |
| CD79B | EAW94229.1 | 18 |
| CD79B | EAW94230.1 | 19 |

Another aspect of the invention provides a population of cells comprising at least one of any of the inventive cells described herein with respect to other aspects of the invention. The population of cells can be a heterogeneous population comprising the cell expressing the modified CD3 subunit chain or non-CD3 subunit chain described herein, in addition to at least one other cell (e.g., a T cell, e.g., a B cell, a macrophage, or a neutrophil) which does not express the modified CD3 subunit chain or non-CD3 subunit chain. Alternatively, the population of cells can be a substantially homogeneous population, in which the population comprises mainly of (e.g., consisting essentially of) cells expressing the modified CD3 subunit chain or non-CD3 subunit chain. The population also can be a clonal population of cells, in which all cells of the population are clones of a single cell expressing the modified CD3 subunit chain or non-CD3 subunit chain, such that all cells of the population express the modified CD3 subunit chain or non-CD3 subunit chain.

The inventive cells and populations thereof can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

The inventive cells and populations thereof can be formulated into a composition, such as a pharmaceutical composition. In this regard, an aspect of the invention provides a pharmaceutical composition comprising any of the cell(s) described herein and a pharmaceutically acceptable carrier. The inventive pharmaceutical compositions containing any of the inventive populations of cells can comprise more than one inventive cell, e.g., a T cell expressing the modified CD3 subunit chain or non-CD3 subunit chain and a TCR negative cell expressing the modified CD3 subunit chain or non-CD3 subunit chain. Alternatively, the pharmaceutical composition can comprise inventive cell(s) in combination with another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive cell(s) under consideration. Methods for preparing administrable compositions are known or apparent to those skilled in the art and are described in more detail in, for example, Remington: The Science and Practice of Pharmacy, 23^{rd} Ed., Pharmaceutical Press (2020). It is preferred that the pharmaceutically acceptable carrier be one which has no detrimental side effects or toxicity under the conditions of use.

The choice of carrier will be determined in part by the particular cell(s), as well as by the particular method used to administer the inventive particular cell(s). Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the particular inventive cell(s), and in certain instances, a particular route can provide a more immediate and more effective response than another route.

Preferably, the inventive particular cell(s) is/are administered by injection, e.g., intravenously. The pharmaceutically acceptable carrier for the cell(s) for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an aspect, the pharmaceutically acceptable carrier is supplemented with human serum albumen.

For purposes of the invention, the amount or dose (e.g., numbers of cells) of the inventive cell(s) administered should be sufficient to effect, e.g., a therapeutic or prophylactic response, in the subject over a reasonable time frame. For example, the dose of the inventive cell(s) should be sufficient to treat or prevent a condition or enhance an antigen-specific immune response in a subject in a period of from about 2 hours or longer, e.g., 12 to 24 or more hours, from the time of administration. In certain aspects, the time period could be even longer. The dose will be determined by the efficacy of the particular inventive cell(s) and the condition of the subject (e.g., human), as well as the body weight of the subject (e.g., human) to be treated.

Many assays for determining an administered dose are known in the art. For purposes of the invention, an assay, which comprises comparing the extent to which target cells are lysed or IFN-γ is secreted by the inventive cell(s) upon administration of a given dose of such cells to a subject among a set of subjects of which each is given a different dose of the T cells, could be used to determine a starting dose to be administered to a subject. The extent to which target cells are lysed or IFN-γ is secreted upon administration of a certain dose can be assayed by methods known in the art.

The dose of the inventive cell(s) also will be determined by the existence, nature and extent of any adverse side effects that might accompany the administration of a particular inventive cell(s). Typically, the attending physician will decide the dosage of the inventive cell(s) with which to treat each individual patient, taking into consideration a variety of factors, such as age, body weight, general health, diet, sex, inventive cell(s) to be administered, route of administration, and the severity of the cancer or infectious disease being treated. In an aspect in which a population of cells is administered, the number of cells administered per infusion may vary, e.g., from about 1×10⁶ to about 1×10¹² cells or more. In certain aspects, fewer than 1×10⁶ cells may be administered.

### Methods of Treating or Preventing a Condition in a Subject and Methods of Enhancing an Antigen-Specific Immune Response in a Subject

It is contemplated that the inventive cell(s) and pharmaceutical compositions can be used in methods of treating or preventing a condition in a subject. Without being bound to a particular theory or mechanism, the inventive cell(s) are believed to enhance an immune response against a target cell expressing a cancer antigen, an autoimmune disease self-antigen, or infectious disease antigen. In this regard, an aspect of the invention provides a method of treating or preventing a condition in a subject in a subject, comprising administering to the subject any of the pharmaceutical compositions, cells, or populations of cells described herein, in an amount effective to treat or prevent the condition in the subject, wherein the cell expresses one or more of:

(a) a modified CD3 subunit chain or non-CD3 subunit chain comprising at least one ITAM deletion;

(b) a modified CD3 subunit chain or non-CD3 subunit chain comprising at least one exogenous intracellular hematopoietic cell signaling domain; and

(c) a modified CD3 subunit chain or non-CD3 modified ITAM containing subunit chain comprising at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈ and X₉ is, independently, any amino acid residue;
each one of X₅ is, independently, any amino acid residue;
m is 6, 7, 8, 9, 10, 11, or 12; and
   wherein the modified CD3 subunit chain is not comprised in a CAR.
   The cell, CD3 subunit chain or non-CD3 modified ITAM containing subunit and modified ITAM comprising the amino acid sequence of Formula I are as described herein with respect to other aspects of the invention.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of condition in a subject. Furthermore, the treatment or prevention provided by the inventive method can include treatment or prevention of one or more conditions or symptoms of the condition being treated or prevented. For example, treatment or prevention can include promoting the regression of a tumor. Also, for purposes herein, “prevention” can encompass delaying the onset of the condition, or a symptom or condition thereof. Alternatively or additionally, “prevention” may encompass preventing or delaying the recurrence of condition, or a symptom or condition thereof.

In an aspect of the invention, the condition is cancer. The cancer can be any cancer, including any of leukemia (e.g., B cell leukemia), sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, uterine cervical cancer, gastrointestinal carcinoid tumor, hypopharynx cancer, larynx cancer, liver cancer, lung cancer, malignant mesothelioma, melanoma, multiple myeloma, nasopharynx cancer, oropharynx, ovarian cancer, pancreatic cancer, penis, peritoneum, rectum, omentum, and mesentery cancer, pancreas, pharynx cancer, prostate cancer, rectal cancer, renal cancer, small intestine cancer, soft tissue cancer, stomach cancer, testicular cancer, thyroid cancer, ureter cancer, vagina, and urinary bladder cancer.

In an aspect of the invention, the condition is an infectious disease. The term “infectious disease” as used herein refers to any disease that results from infection with an agent. Such agents may include, for example, bacteria, viruses, fungi, and parasites, as described herein.

In an aspect of the invention, the infectious disease is a viral disease. The viral disease may affect any part of the body. The viral disease may be caused by any of the viruses described herein with respect to the viral antigen. In an aspect of the invention, the viral disease is selected from the group consisting of influenza, pneumonia, herpes, hepatitis, hepatitis A, hepatitis B, hepatitis C, chronic fatigue syndrome, sudden acute respiratory syndrome (SARS), COVID-19, gastroenteritis, enteritis, carditis, encephalitis, bronchiolitis, respiratory papillomatosis, meningitis, mononucleosis, and a pulmonary viral disease (e.g., pneumonia).

In an aspect of the invention, the infectious disease is a bacterial disease. The bacterial disease may affect any part of the body. The bacterial disease may be caused by any of the bacteria described herein with respect to the bacterial antigen. In an aspect of the invention, the bacterial disease is meningitis, tetanus, tuberculosis, gonorrhea, chlamydia, cholera, leprosy, tuberculosis, plague, syphilis, typhus, diphtheria, typhoid, dysentery, pneumonia, anthrax, listeriosis, and gastroenteritis.

In an aspect of the invention, the infectious disease is a fungal disease. The fungal disease may affect any part of the body. The fungal disease may be caused by any of the fungi described herein with respect to the fungal antigen. In an aspect of the invention, the fungal disease is selected from the group consisting of cryptococcosis, aspergillosis, coccidioidomycosis (valley fever), histoplasmosis, blastomycosis, and pneumocystis pneumonia.

In an aspect of the invention, the infectious disease is a parasitic disease. The parasitic disease may affect any part of the body. The parasitic disease may be caused by any of the parasites described herein with respect to the parasitic antigen. In an aspect of the invention, the parasitic disease is selected from the group consisting of trichomoniasis, giardiasis, cryptosporidiosis, toxoplasmosis, and malaria.

In an aspect of the invention, the condition is an autoimmune disease. Examples of autoimmune disease include, but are not limited to, rheumatoid arthritis, systemic lupus erythematosus (lupus), inflammatory bowel disease (IBD), multiple sclerosis (MS), type 1 diabetes mellitus, Guillain-Barre syndrome, chronic inflammatory demyelinating polyneuropathy, and psoriasis.

Another aspect of the invention provides a method of enhancing an antigen-specific immune response in a subject, comprising administering a cell, or a population thereof, to the subject, in an amount effective to enhance the antigen-specific immune response in the subject, wherein the cell expresses one or more of:
(a) a modified CD3 subunit chain or non-CD3 subunit chain comprising at least one ITAM deletion;
(b) a modified CD3 subunit chain or non-CD3 subunit chain comprising at least one exogenous intracellular hematopoietic cell signaling domain; and
(c) a modified CD3 subunit chain or non-CD3 modified ITAM containing subunit chain comprising at least one modified ITAM comprising an amino acid sequence of Formula I:

X₁X₂X₃X₄(X₅)ₘX₆X₇X₈X₉ (Formula I),

wherein:
each of X₁ and X₆ is, independently, any amino acid residue, with the proviso that at least one of X₁ and X₆ is not tyrosine;
each of X₂, X₃, X₄, X₇, X₈ and X₉ is, independently, any amino acid residue;
each one of X₅ is, independently, any amino acid residue;
m is 6, 7, 8, 9, 10, 11, or 12; and
   wherein the modified CD3 subunit chain is not comprised in a CAR.
   The antigen, cell, modified CD3 subunit chain, non-CD3 subunit chain, and modified ITAM comprising the amino acid sequence of Formula I are as described herein with respect to other aspects of the invention.

An antigen-specific immune response is enhanced in accordance with the invention if the immune response to a given antigen is greater, quantitatively or qualitatively, after administration of any of the inventive cell(s) comprising the modified CD3 subunit chain or non-CD3 subunit chain as compared to the immune response in the absence of the administration of cell(s) comprising the modified CD3 subunit chain or non-CD3 subunit chain. A quantitative increase in an immune response encompasses an increase in the magnitude or degree of the response. The magnitude or degree of an immune response can be measured on the basis of any number of known parameters, such as a reduction in the size of a primary tumor or tumor metastases, an increase in the level of antigen-specific cytokine production (cytokine concentration), an increase in the number of lymphocytes activated (e.g., proliferation of antigen-specific lymphocytes) or recruited to a tumor or site of infection, and/or an increase in the production of antigen-specific antibodies (antibody concentration), etc. A qualitative increase in an immune response encompasses any change in the nature of the immune response that renders it more effective at combating a given antigen or disease. Other examples of qualitative increases in an immune response include a shift towards effector-memory or memory-type T cells providing extended efficacy or a reduction in the percentage of “exhausted” antigen-specific T cells that express markers of inactivity and are functionally compromised. Qualitative and quantitative enhancements in an immune response can occur simultaneously, and are not mutually exclusive.

In an aspect of the inventive methods, the cell expresses an antigen-specific receptor. The antigen-specific receptor may be as described herein with respect to other aspects of the invention.

In an aspect of the inventive methods, the antigen is a cancer antigen, an autoimmune disease self-antigen, or infectious disease antigen. The cancer antigen, autoimmune disease self-antigen, and infectious disease antigen may be as described herein with respect to other aspects of the invention.

In an aspect of the inventive methods, the cell is any of the inventive cells described herein with respect to other aspects of the invention.

For purposes of the inventive methods, wherein populations of cells are administered, the cells can be cells that are allogeneic or autologous to the subject. Preferably, the cells are autologous to the subject to avoid graft vs host disease or graft rejection.

The subject referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

### Methods of Making the Cells Expressing a Modified CD3 Subunit Chain or Non-CD3 Subunit Chain I

The inventive cells and populations of cells described herein may be made in any of a variety of different ways including, but not limited to, the methods described herein.

An aspect of the invention provides a method of making any of the inventive cell(s) described herein comprising modifying a cell to comprise a nucleotide sequence encoding the modified CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain. “Nucleic acid,” as used herein, as used herein, includes “polynucleotide,” “oligonucleotide,” and “nucleic acid molecule,” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

The nucleic acids may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook, Molecular Cloning: A Laboratory Manual, 4^{th} ed., Cold Spring Harbor Press, Cold Spring Harbor, NY (2012). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, β-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, 13-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from any of a variety of commercial entities.

In an aspect of the invention, the nucleic acid comprises a codon-optimized nucleotide sequence encoding any of the modified CD3 subunit chains, non-CD3 subunit chains comprising at least one ITAM deletion, non-CD3 subunit chains comprising at least one exogenous intracellular hematopoietic cell signaling domain, or non-CD3 modified ITAM containing chains described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

In an aspect of the invention, modifying the cell comprises introducing the nucleotide sequence encoding the modified CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain into a CD3 subunit positive cell or non-CD3 ITAM containing subunit positive cell, respectively. The CD3 subunit positive cell may be any cell which expresses a CD3 subunit, including any of the cells described herein with respect to other aspects of the invention. For example, the CD3 subunit positive cell may be a TCR positive cell which expresses a complete TCR complex. In other aspects, the CD3 subunit positive cell may be a TCR negative cell which expresses a CD3 subunit but not a complete TCR complex, for example, the TCR negative cells described herein with respect to other aspects of the invention.

In an aspect of the invention, modifying the cell comprises introducing the nucleotide sequence encoding the non-CD3 modified ITAM containing subunit chain comprising the amino acid sequence of Formula I into a cell that naturally expresses the non-CD3 ITAM containing subunit. The non-CD3 ITAM containing subunit positive cell may be any cell which expresses that subunit, including any of the cells described herein with respect to other aspects of the invention in addition to a B cell, macrophage, granulocyte or dendritic cell.

In another aspect of the invention, modifying the cell comprises introducing the nucleotide sequence encoding the modified CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain into a pluripotent stem cell or multipotent stem cell. Pluripotent stem cells have the capacity to give rise to any of the three germ layers: endoderm, mesoderm, and ectoderm. Pluripotent stem cells may comprise, for example, stem cells, e.g., embryonic stem cells, nuclear transfer derived embryonic stem cells, induced pluripotent stem cells (iPSC), etc. The pluripotent stem cells may have a stem cell phenotype including (i) the ability to self-renew and (ii) pluripotency. For example, the pluripotent stem cells, e.g., iPSCs, may be morphologically indistinguishable from embryonic stem cells (ESCs). For example, the induced pluripotent stem cells, e.g., iPSCs, may have any one or more of a round shape, large nucleolus and small volume of cytoplasm. Alternatively or additionally, the pluripotent stem cells, e.g., iPSCs, may be any one or more of mitotically active, actively self-renewing, proliferating, and dividing. Alternatively or additionally, the pluripotent stem cells, e.g., iPSCs, may express any one or more of a variety of pluripotency-associated genes. Pluripotency-associated genes may include, but are not limited to, Oct-3/4, Sox2, Nanog, GDF3, REX1, FGF4, ESG1, DPPA2, DPPA4, hTERT and SSEA1.

Multipotent stem cells are cells that have the capacity to self-renew by dividing and to develop into multiple specialized cell types present in a specific tissue or organ. Multipotent stem cells produce cells of a closely related family of cells. In an aspect of the invention, the multipotent stem cells are hematopoietic stem cells.

The method may further comprise differentiating the pluripotent stem cell or multipotent stem cell with the introduced nucleotide sequence into a cell which expresses the CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain, respectively. In this regard, the method may comprise culturing the pluripotent stem cells or multipotent stem cells for a time and under conditions sufficient to differentiate the pluripotent stem cells into a CD3 subunit positive cell or cell that expresses a non-CD3 ITAM containing chain including any of the CD3 subunit positive cells, or non-CD3 ITAM containing chain positive cells described herein with respect to other aspects of the invention. Methods of differentiating pluripotent stem cells and multipotent stem cells into a variety of cell types are known in the art.

In an aspect of the invention, modifying the cell comprises introducing the nucleotide sequence encoding the modified CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain into the cell using transfection, transformation, transduction, electroporation, a transposon, or a genome editing technique. In an aspect of the invention, the genome editing technique to introduce the nucleotide sequence uses a zinc finger nuclease, transcription activator-like effector nuclease (TALENs), a CRISPR/Cas system, or engineered meganuclease.

In an aspect of the invention, the method may further comprise suppressing expression of an endogenous, wild-type CD3 subunit chain corresponding to the modified CD3 subunit chain, suppressing expression of an endogenous, wild-type non-CD3 ITAM containing subunit chain corresponding to the non-CD3 modified ITAM containing subunit chain, suppressing expression of an endogenous, wild-type non-CD3 ITAM containing subunit chain corresponding to the non-CD3 subunit chain comprising at least one ITAM deletion, or suppressing expression of an endogenous, wild-type non-CD3 subunit chain corresponding to the non-CD3 subunit chain comprising at least one exogenous intracellular hematopoietic cell signaling domain. Suppressing expression of the endogenous, wild-type subunit chains may be carried out in a variety of different ways. In an aspect of the invention, suppressing expression of the endogenous, wild-type subunit chains may be carried out using a zinc finger nuclease, transcription activator-like effector nuclease (TALENs), a CRISPR/Cas system, engineered meganuclease, or RNA interference.

Genome editing techniques can modify gene expression in a target cell by inserting, replacing, or removing DNA in the genome using an artificially engineered nuclease. Examples of such nucleases may include zinc finger nucleases (ZFNs) (Gommans et al., J. Mol. Biol., 354(3): 507-519 (2005)), transcription activator-like effector nucleases (TALENs) (Zhang et al., Nature Biotechnol., 29: 149-153 (2011)), the CRISPR/Cas system (Cheng et al., Cell Res., 23: 1163-71 (2013)), and engineered meganucleases (Riviere et al., Gene Ther., 21(5): 529-32 (2014)). The nucleases create specific double-stranded breaks (DSBs) at targeted locations in the genome, and use endogenous mechanisms in the cell to repair the induced break by homologous recombination (HR) and nonhomologous end-joining (NHEJ). Such techniques may be used to suppress expression of the endogenous, wild-type subunit chains.

In an aspect of the invention, the method further comprising assembling the modified CD3 subunit chain with further CD3 subunit chains, assembling the non-CD3 subunit chain comprising at least one ITAM deletion with further non-CD3 ITAM containing subunit chains, assembling the non-CD3 modified ITAM containing chain with further non-CD3 ITAM containing subunit chains, assembling the non-CD3 subunit chain comprising at least one exogenous intracellular hematopoietic cell signaling domain with further non-CD3 subunit chains, or assembling the modified CD3 subunit or non-CD3 modified ITAM containing chain with further CD3 ITAM containing subunit chains. In this regard, the modified CD3 subunit chain may assemble with further CD3 subunit chains to form dimers such as, for example, a CD3gamma/CD3epsilon dimer, a CD3delta/CD3epsilon dimer, or a CD3zeta/CD3zeta dimer. Alternatively the modified CD3 subunit chain may assemble with further CD3 subunit chains to form a complete TCR complex composed of eight subunits: a TCRalpha/TCRbeta dimer, a CD3gamma/CD3epsilon dimer, a CD3delta/CD3epsilon dimer, and a CD3zeta/CD3zeta dimer.

In an aspect of the invention, the method further comprises assembling the modified non-CD3 subunit chain (e.g., non-CD3 modified ITAM containing subunit chain) with endogenous non-CD3 chains that comprise receptors other than the TCR. In this regard, the modified non-CD3 subunit chain (e.g., non-CD3 modified ITAM containing subunit chain) may assemble with another non-CD3 chain to form dimers such as, for example, an CD3zeta/FcepsilonR1gamma dimer included in the NKp30 receptor and NKp46 receptor, and a DAP12/DAP12 dimer in the NKp44 receptor or MR receptors (Lanier, Immunol. Rev., 227(1): 150-60 (2009)).

In an aspect of the invention, the numbers of cells in the population may be rapidly expanded. Expansion of the numbers of cells (e.g., T cells) can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990). In an aspect, expansion of the numbers of T cells is carried out by culturing the T cells with OKT3 antibody, IL-2, and feeder PBMC (e.g., irradiated allogeneic PBMC).

The following examples further illustrate the invention but, of course, should not be construed as in any way limiting its scope.

### Example 1

This example demonstrates that cells expressing a TCR comprising a CD3zeta chain dimer in which all of the ITAM tyrosines (Y) were mutated to phenylalanine (F) provided superior tumoricidal activity in vivo compared to control cells expressing a TCR with a wild-type (WT) CD3zeta chain dimer.

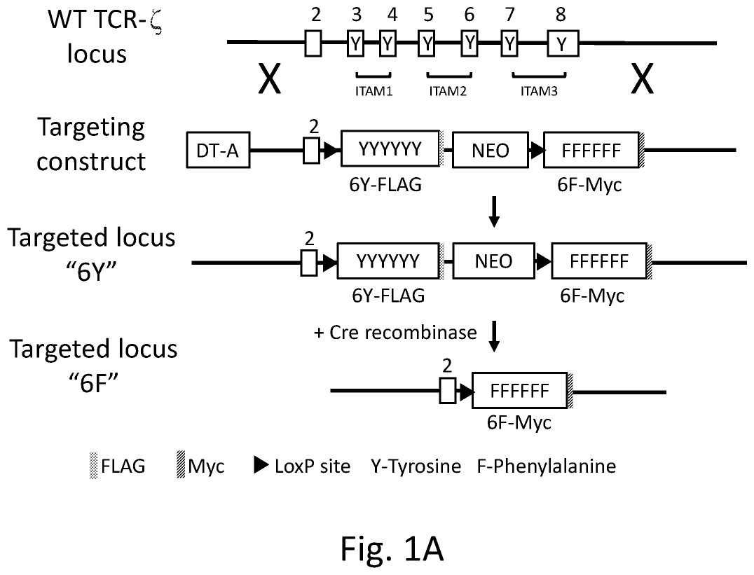

A mouse model was made for studying TCR ITAM function in mature T cells as described in Hwang et al., J. Exp. Med., 209: 1781-1795 (2012). In this mouse model, ITAMs can be inactivated at any point during development, for example, after T cells have fully developed and left the thymus. To this end, a ‘switchable’ mouse model was generated containing a ‘knock-in’ within the CD3zeta locus that expresses only WT “6Y CD3zeta” (6Y/6Y). After Cre-mediated recombination, the mouse model expressed only inactive “6F CD3zeta” (6F/6F) (FIG. 1A). In the inactive “6F CD3zeta,” all of the ITAM tyrosines (Y) were mutated to phenylalanine (F) (FIG. 1B). The TCRs expressed in the “6F CD3zeta” mice contained WT CD3gamma/CD3epsilon and CD3delta/CD3epsilon dimers with intact ITAMs. The TCRs in the 6F CD3zeta mouse contained four functional ITAMs (contributed by CD3gamma/CD3epsilon and CD3delta/CD3epsilon dimers). The remaining six ITAMs within CD3zeta were inactivated (Hwang et al., J. Exp. Med., 209: 1781-1795 (2012); Hwang et al., Nat. Commun., 6: 6982 (2015)). The 6Y and 6F ITAM amino acid sequences are set forth in Table 4.

TABLE 4

| | SEQ ID | |
|---|---|---|
| Name | NO: | Sequence |
| Human | 20 | MKWKALFTAAILQAQLPITEAQSFGLLDPKLCYLLDGILFIY |
| CD3z 6Y | | GVILTALFLRVKFSRSADAPAYQQGQNQLYNELNLGRREE |
| | | YDVLDKRRGRDPEMGGKPQRRKNPQEGLYNELQKDKMAE |
| | | AYSEIGMKGERRRGKGHDGLYQGLSTATKDTYDALHMQA |
| | | LPPR |
| Human | 21 | MKWKALFTAAILQAQLPITEAQSFGLLDPKLCYLLDGILFIY |
| CD3z 6F | | GVILTALFLRVKFSRSADAPAYQQGQNQLFNELNLGRREEF |
| | | DVLDKRRGRDPEMGGKPQRRKNPQEGLFNELQKDKMAEA |
| | | FSEIGMKGERRRGKGHDGLFQGLSTATKDTFDALHMQALP |
| | | PR |
| Mouse | 22 | MKWKVSVLACILHVRFPGAEAQSFGLLDPKLCYLLDGILFI |
| CD3z 6Y | | YGVIITALYLRAKFSRSAETAANLQDPNQLYNELNLGRREE |
| | | YDVLEKKRARDPEMGGKQQRRRNPQEGVYNALQKDKMA |
| | | EAYSEIGTKGERRRGKGHDGLYQGLSTATKDTYDALHMQT |
| | | LAPR |
| Mouse | 23 | MKWKVSVLACILHVRFPGAEAQSFGLLDPKLCYLLDGILFI |
| CD3z 6F | | YGVIITALYLRAKFSRSAETAANLQDPNQLFNELNLGRREE |
| | | FDVLEKKRARDPEMGGKQQRRRNPQEGVFNALQKDKMA |
| | | EAFSEIGTKGERRRGKGHDGLFQGLSTATKDTFDALHMQT |
| | | LAPR |
| Human | 24 | MKWKALFTAAILQAQLPITEAQSFGLLDPKLCYLLDGILFIY |
| CD3z 6A | | GVILTALFLRVKFSRSADAPAYQQGQNQLANELNLGRREE |
| | | ADVLDKRRGRDPEMGGKPQRRKNPQEGLANELQKDKMAE |
| | | AASEIGMKGERRRGKGHDGLAQGLSTATKDTADALHMQA |
| | | LPPR |
| Mouse | 25 | MKWKVSVLACILHVRFPGAEAQSFGLLDPKLCYLLDGILFI |
| CD3z 6A | | YGVIITALYLRAKFSRSAETAANLQDPNQLANELNLGRREE |
| | | ADVLEKKRARDPEMGGKQQRRRNPQEGVANALQKDKMA |
| | | EAASEIGTKGERRRGKGHDGLAQGLSTATKDTADALHMQT |
| | | LAPR |

The tumoricidal activity of CD8⁺ T cells that express TCRs with 6F CD3zeta was assessed by injecting these T cells into mice with melanoma tumors. Tumor cell lines (B16F10 melanoma cells) were previously transduced with vectors that encode a foreign peptide from chicken ovalbumin (OVA) (N4, serving as a pseudo-tumor antigen) or a variant OVA peptide (V4) that binds with lower affinity to the TCR compared to N4. Using N4 and V4, tumor reactivity of T cells to high or low affinity “tumor antigens” could be monitored.

Tumors were generated in mice by subcutaneously injecting the B16F10 melanoma cells that express the high affinity ovalbumin peptide N4 or the low affinity ovalbumin peptide V4 into C57BL/6 mice or T/B cell deficient Rag1^{-/-} mice.

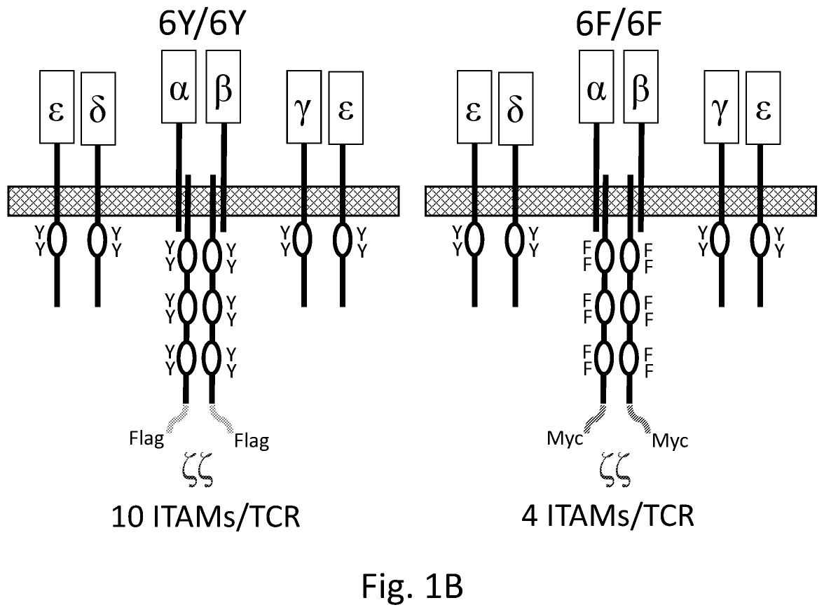

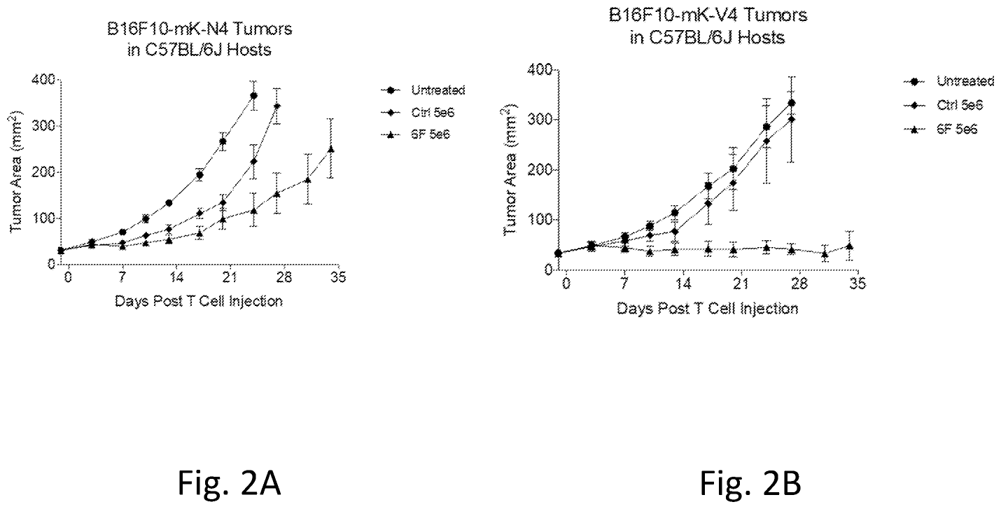

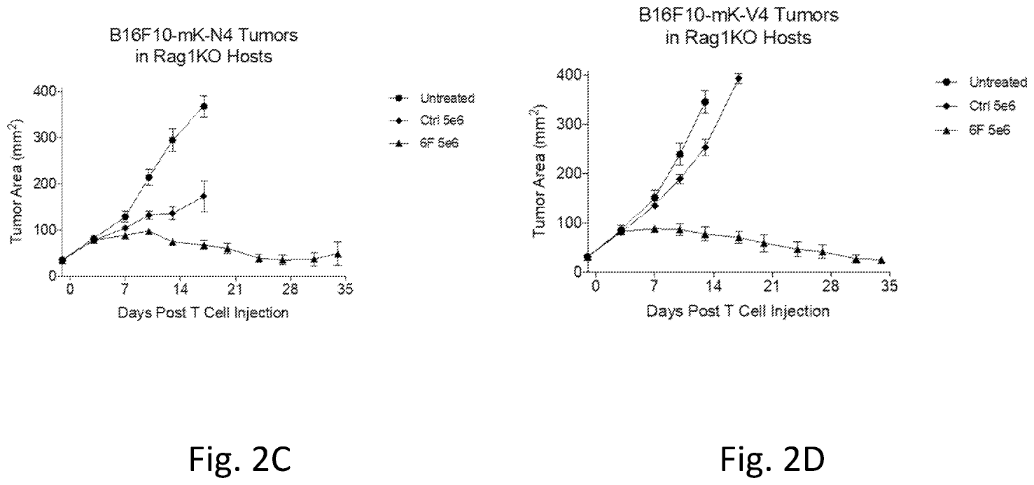

The donor T cells were from OT-1 TCR transgenic mice. The OT-1 TCR transgenic mice expressed transgenic TCRalpha and TCRbeta chains that recognize OVA on all T cells. The B16F10 tumor-bearing mice were injected with OT-1 TCR transgenic 6Y CD3zeta CD8⁺ T cells (control) (5×10⁶) or OT-1 TCR transgenic 6F CD3zeta CD8⁺ T cells (5×10⁶). The latter T cells express 6F CD3zeta all through their development (the Cre transgene was expressed in the germline to make 6F CD3zeta expression constitutive). Untreated (un-injected with donor T cells) mice injected with B16F10 melanoma cells served as a control. Tumor area was measured on the days indicated in FIGS. 2A-2D. Five mice were included in each group.

Based on previous data, the prediction was that the OT-1 TCR transgenic 6F CD3zeta CD8⁺ T cells would be less efficient than control, OT-1 TCR transgenic 6Y CD3zeta CD8⁺ T cells in killing OVA peptide-expressing melanoma cells because TCRs that contain 6F CD3zeta have fewer ITAMs compared to 6Y CD3zeta TCRs (4 ITAMs/TCR vs 10 ITAMs/TCR. Unexpectedly, it was found that OT-1 TCR transgenic 6F CD3zeta CD8⁺ T cells were far superior to OT-1 TCR transgenic 6Y CD3zeta CD8⁺ T cells in controlling tumor growth. This superiority was most evident with melanoma cells that expressed the lower affinity V4 peptide antigen (FIGS. 2A-2D).

### Example 2

This example demonstrates that cells expressing a TCR comprising a CD3zeta chain dimer in which all of the ITAM tyrosines (Y) were mutated to phenylalanine (F) provide higher in vitro killing potency toward tumor cells expressing low affinity antigens (V4) compared to their 6Y CD3zeta counterparts.

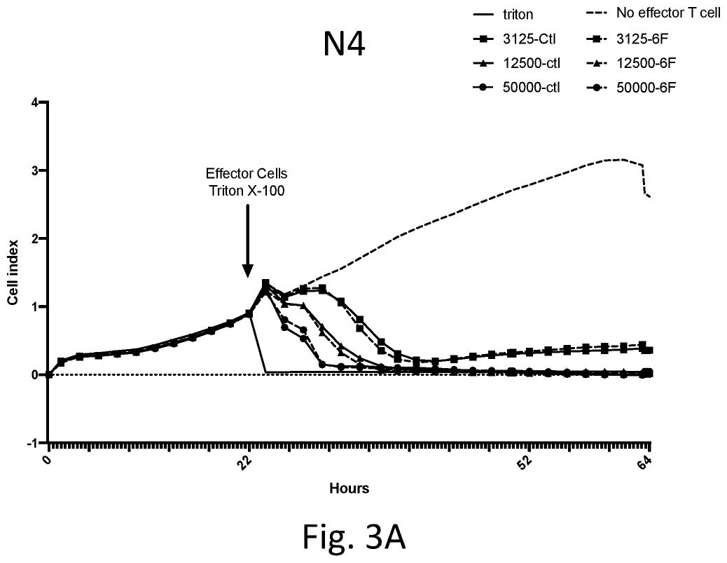

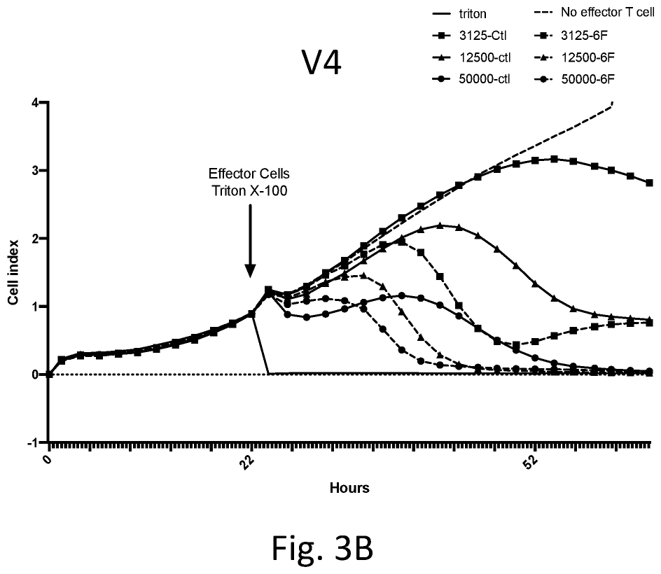

In vitro T-cell mediated cytolysis of melanoma tumor cell lines by OT-1 TCR transgenic CD8⁺ T cells expressing TCRs containing 6Y CD3zeta or 6F CD3zeta was determined by an ACEA XCELLIGENCE Real Time Cell Analyzer instrument (Agilent Technologies, Inc., Santa Clara, CA). B16F10 melanoma cells that express the high affinity ovalbumin peptide N4 (FIG. 3A) or the low affinity ovalbumin altered peptide V4 (FIG. 3B), (as described in Example 1) were co-cultured with variable numbers of CD8⁺ T cells from OT-1 TCR transgenic 6Y/6Y mice of Example 1 (control), or OT-1 TCR transgenic 6F/6F mice of Example 1. TRITON x-100 surfactant (MilliporeSigma, St. Louis, MO) was used as a positive control for lysis. The OT-1 TCR transgenic 6F/6F T cells express 6F CD3zeta all through their development, as described in Example 1.

The results of the in vitro experiments of 6Y vs 6F OT-1 TCR cytolytic activity against tumor cells was in line with the in vivo data obtained in the Experiment of Example 1. The OT-1 TCR transgenic CD3zeta 6F expressing cells displayed much higher in vitro killing potency toward tumor cells, particularly those expressing low affinity antigens (V4) compared to their OT-1 TCR transgenic 6Y CD3zeta counterparts (FIG. 3B).

### Example 3

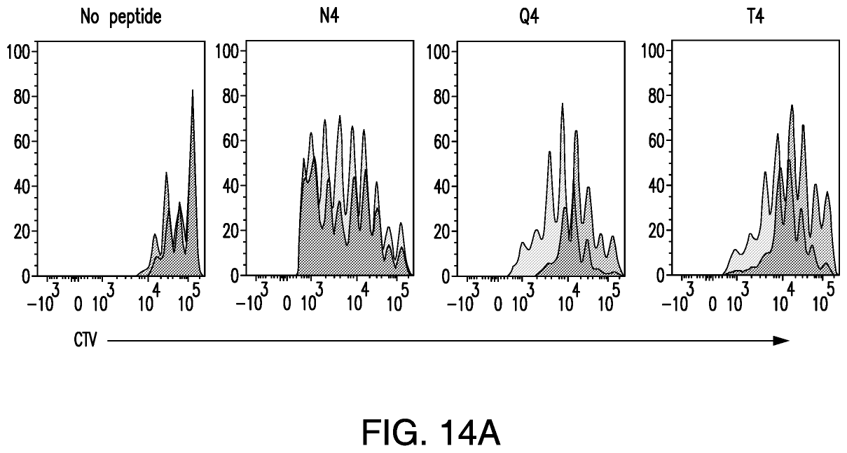

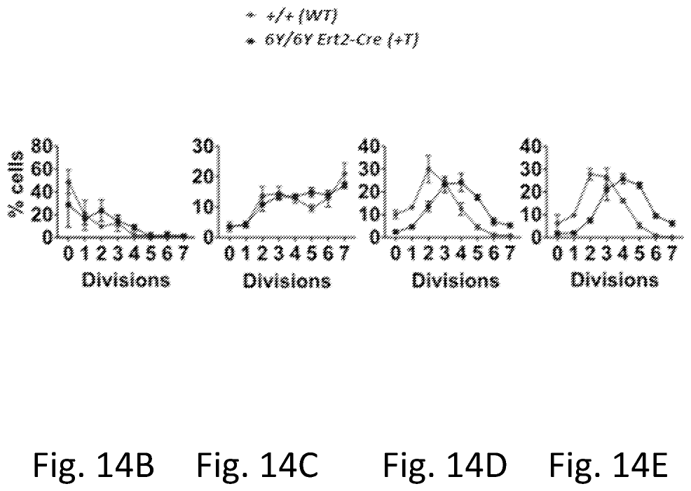

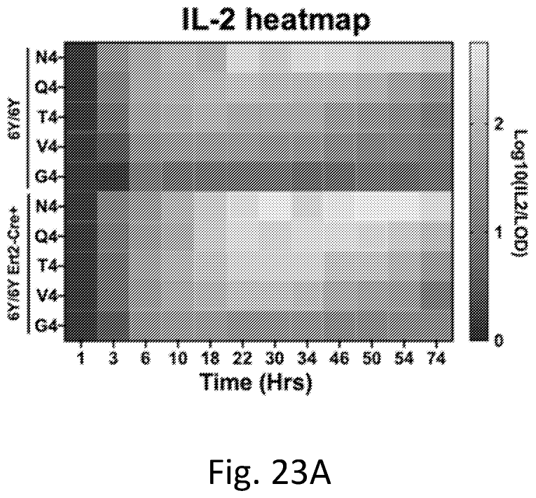

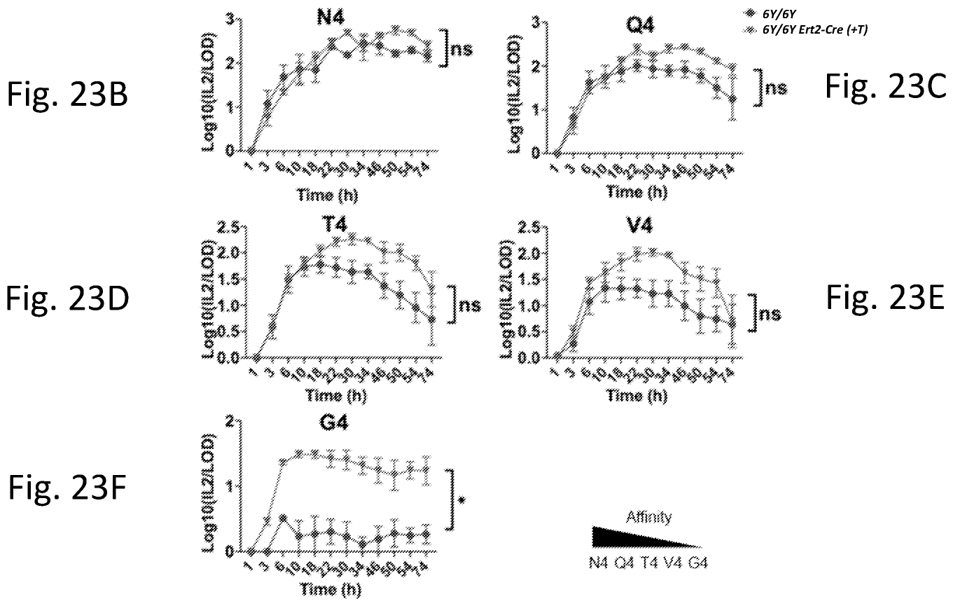

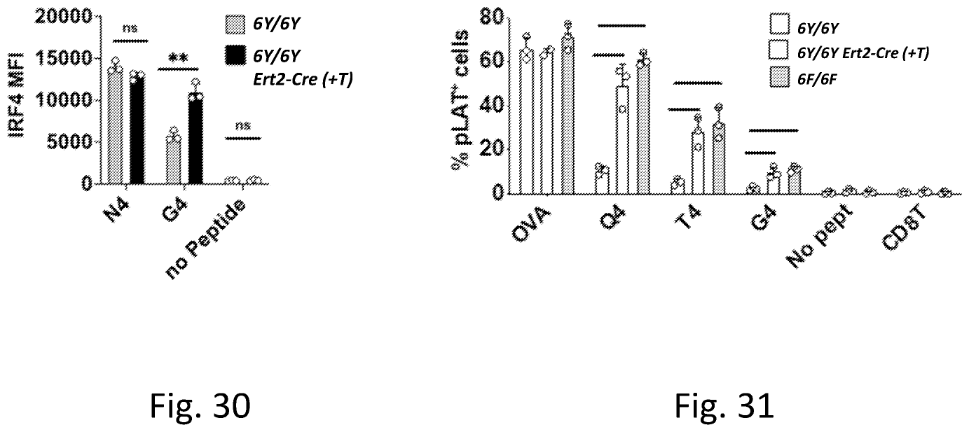

This example (FIG. 14A-E; FIG. 23A-E; FIG. 30, 31) demonstrates that ‘acutely switched’ cells expressing a TCR comprising a CD3zeta chain dimer in which all of the ITAM tyrosines (Y) were mutated to phenylalanine (F) transduce ‘stronger’ signals (as measured by cytokine production and expression of activation markers) than control, 6Y CD3zeta TCRs in response to low affinity antigens.

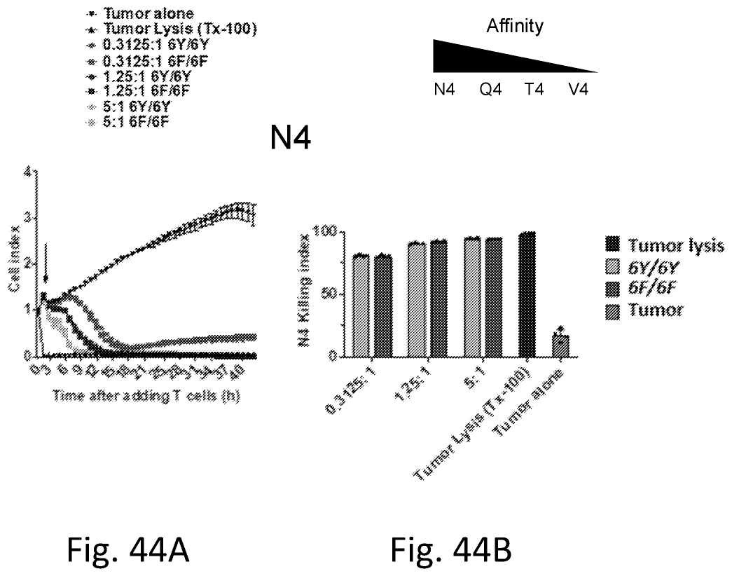

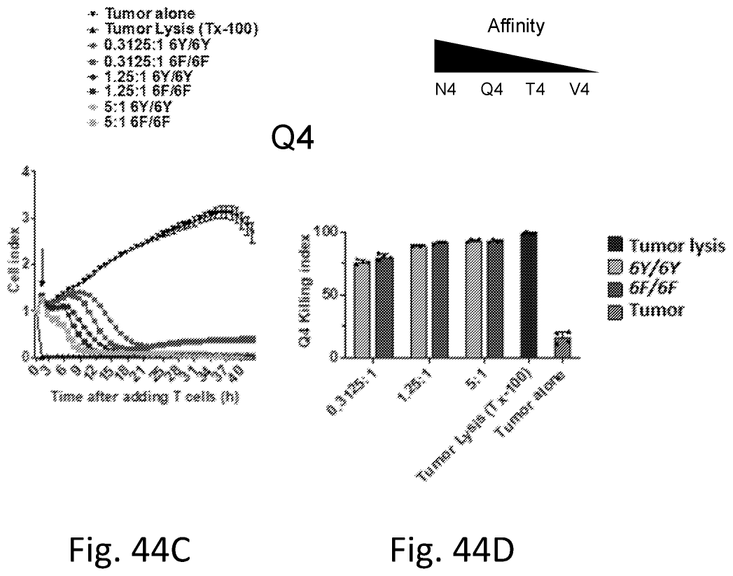

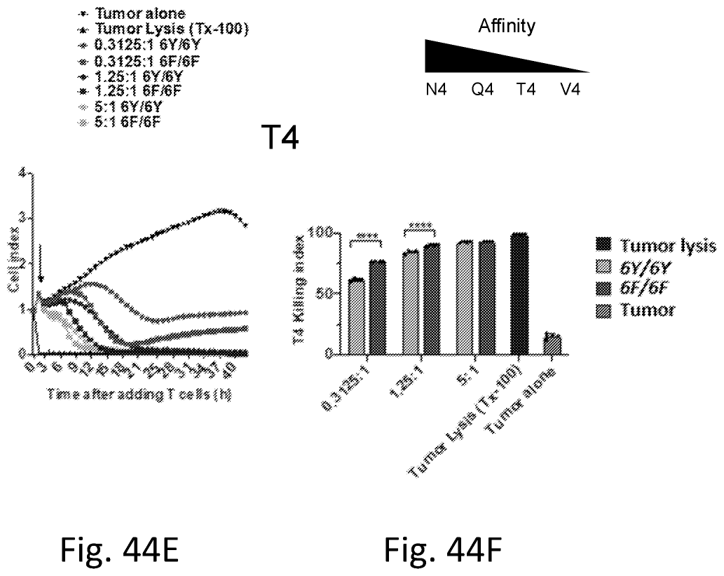

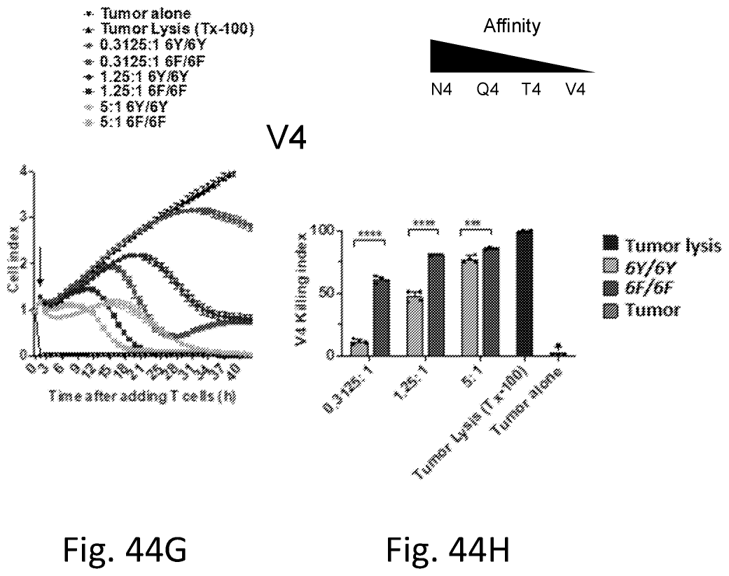

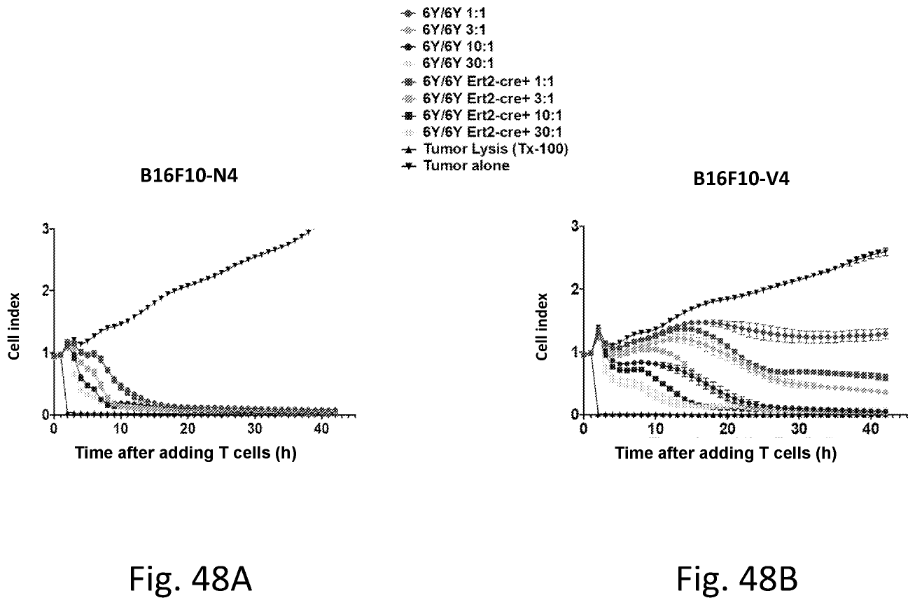

B16F10 melanoma cells that express the high affinity ovalbumin peptide N4 or the low affinity ovalbumin altered peptide V4 (as described in Example 1) were co-cultured with CD8⁺ OT-1 TCR transgenic 6Y/6Y T cells or OT-1 TCR transgenic 6F/6F T cells. OT-1 TCR transgenic 6F/6F T cells were generated by treating OT-1 TCR transgenic ERT2Cre+CD3zeta6Y/6Y T cells with tamoxifen to induce the switch from 6Y to 6F (FIG. 44A-H; FIG. 48A,B).

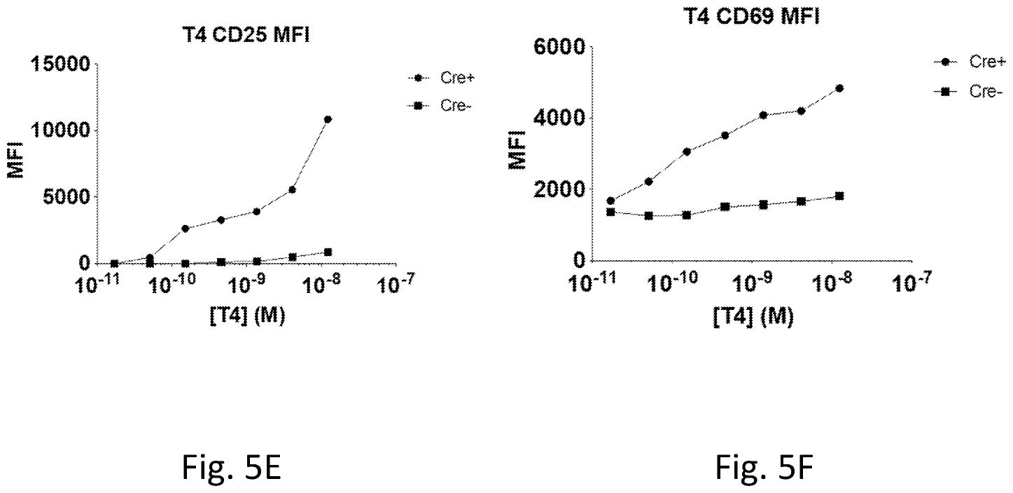

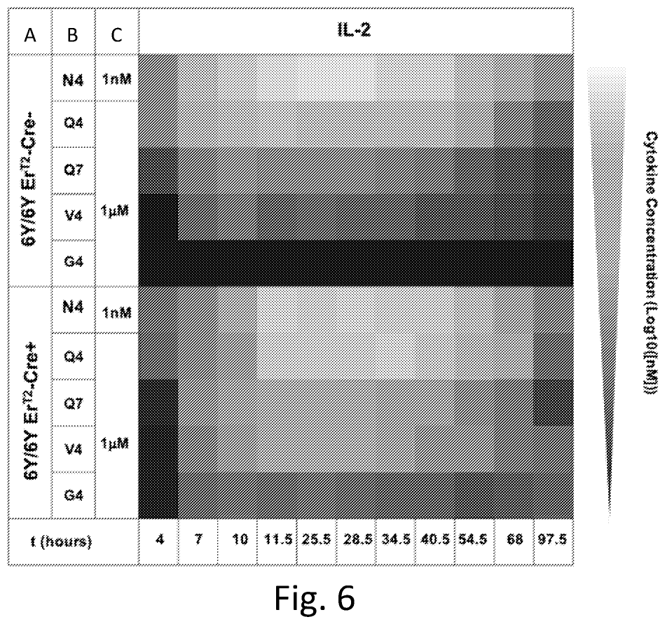

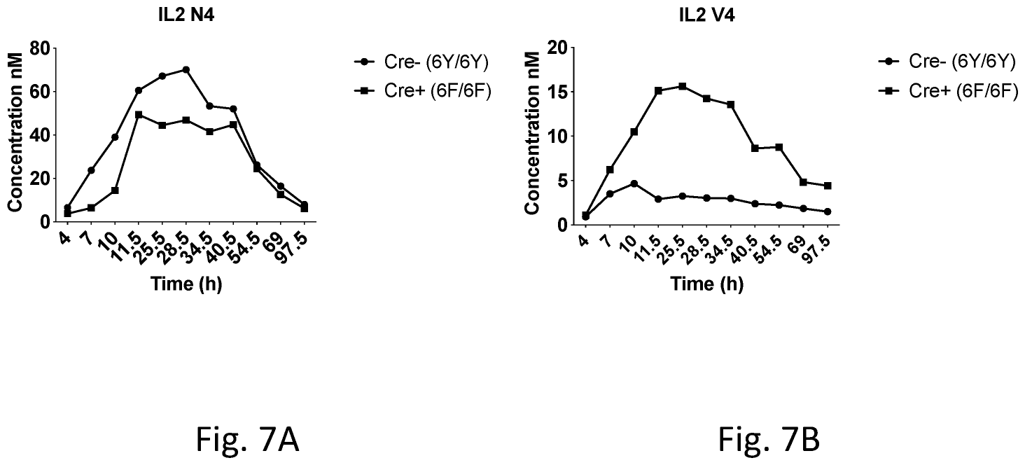

In vitro assessment of TCR signaling intensity measured by cytokine production (FIGS. 6 & 7A-7B) confirmed that 6F CD3zeta TCRs transduce ‘stronger’ signals than control, 6Y CD3zeta TCRs especially in response to low affinity antigens.

### Example 4

This example demonstrates the increased signaling intensity of acutely switched (6Y to 6F) 6F CD3zeta TCRs compared to 6Y CD3zeta TCRs.

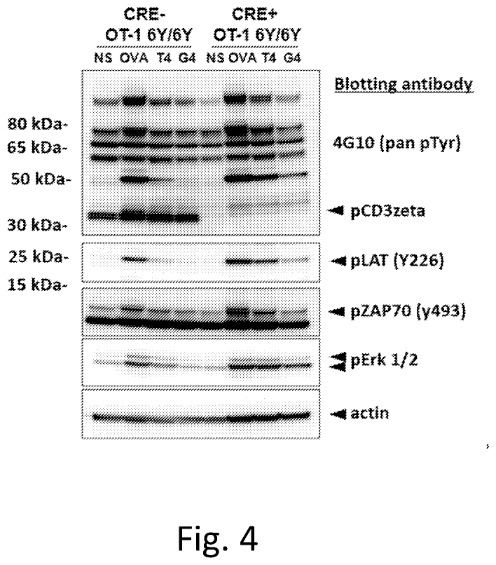

CD8⁺ T cells from OT-1 TCR transgenic 6Y/6Y CD3zeta mice (CRE; OT-1 6Y/6Y) and ERT2CRE+OT-1 TCR transgenic 6Y/6Y mice (CRE+; OT-1 6Y/6Y) were treated with tamoxifen in vitro to induce 6Y to 6F switch in the CRE+ but not in the CRE-cells. T cells were then stimulated with MHC-1 tetramers that contained the indicated peptides (OVA, T4, G4) or left unstimulated (NS). Cells were lysed and run on SDS-PAGE gels then blotted onto nitrocellulose membranes. Membranes were blotted with the blotting antibody indicated in FIG. 4. As shown in FIG. 4, 4G10 (pan pTyr) recognizes all tyrosine phosphorylated proteins. A pCD3zeta band is only detected in cells that express 6Y CD3zeta (FIG. 4). Enhanced generation of pLAT, pZAP70, and pERK1/2 (all downstream effectors of TCR signaling) was observed in stimulated OT-1 6F/6F CD8⁺ T cells (CRP; OT-1 6Y/6Y) particularly after stimulation with weak agonist peptides (T4, G4) (FIG. 4). Actin blot was done as a loading control.

### Example 5

This example demonstrates the enhanced activation of acutely 6Y-6F switched OT-1 TCR transgenic CD8⁺ T cells.

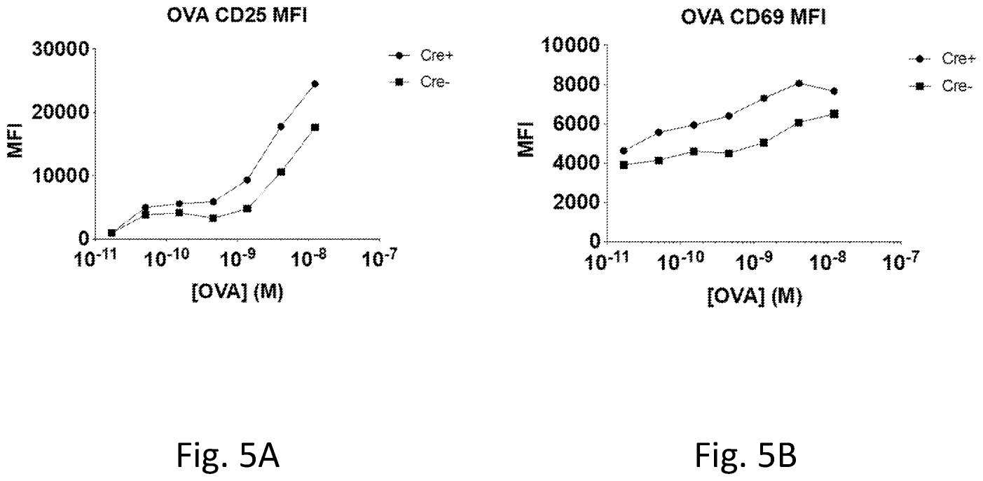

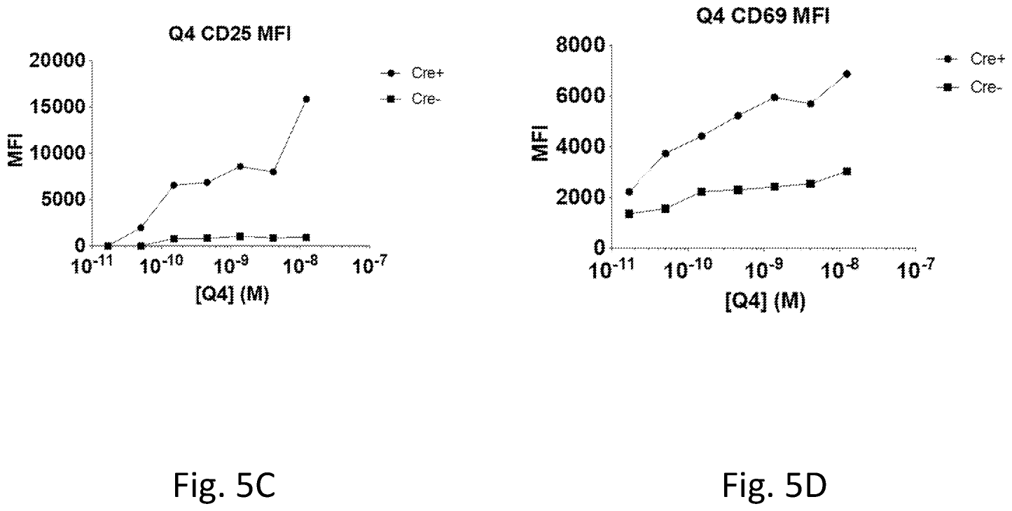

CD8⁺ T cells were purified from ERT2-Cre⁺; OT-1 TCR transgenic 6Y/6Y (Cre⁺) and ERT2-Cre−; OT-1 TCR transgenic 6Y/6Y (Cre⁻) mice then treated with tamoxifen to induce switch of CD3zeta from 6Y to 6F in the ERT2Cre⁺ but not in the ERT2Cre⁻ cells. T cells were then stimulated with antigen presenting cells pulsed with high (OVA), med (Q4) or low (T4) affinity peptides at the concentrations shown in FIGS. 5A-F. T cells were analyzed for expression of the activation markers CD25 (FIGS. 5A, 5C, and 5E) or CD69 (FIGS. 5B, 5D, and 5F) by flow cytometry after 24 hrs. Enhanced expression of the activation markers CD25 and CD69 was especially notable with the switched (Cre⁺) CD8⁺ T cells responding to med (Q4) or low (T4) affinity peptides (FIGS. 5C-5F).

### Example 6

This example demonstrates that post-selection inactivation of CD3zeta ITAMs phenocopies germline CD3zeta ITAM inactivation. (FIGS. 30-32)

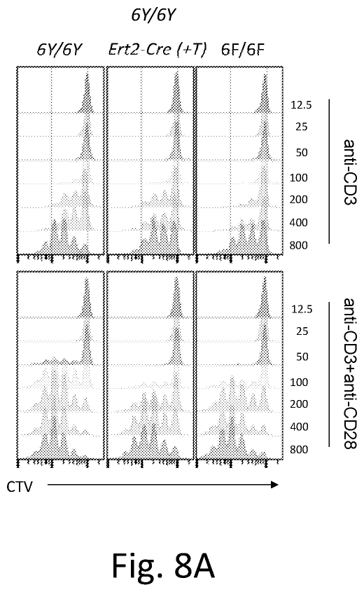

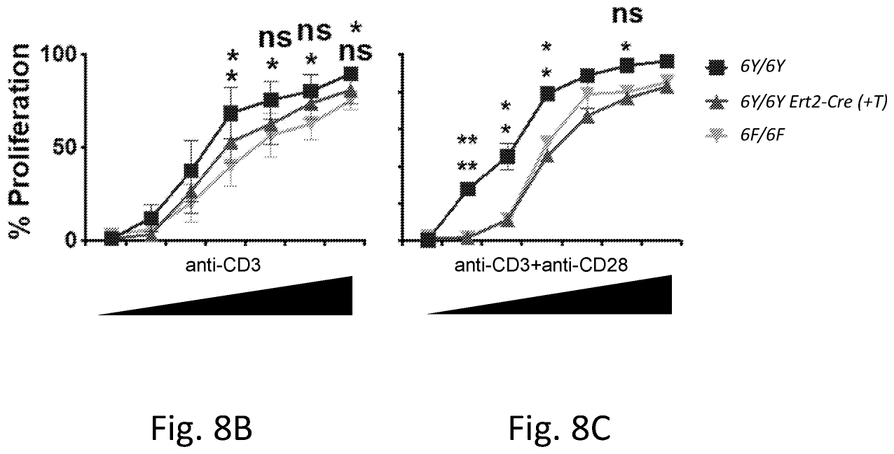

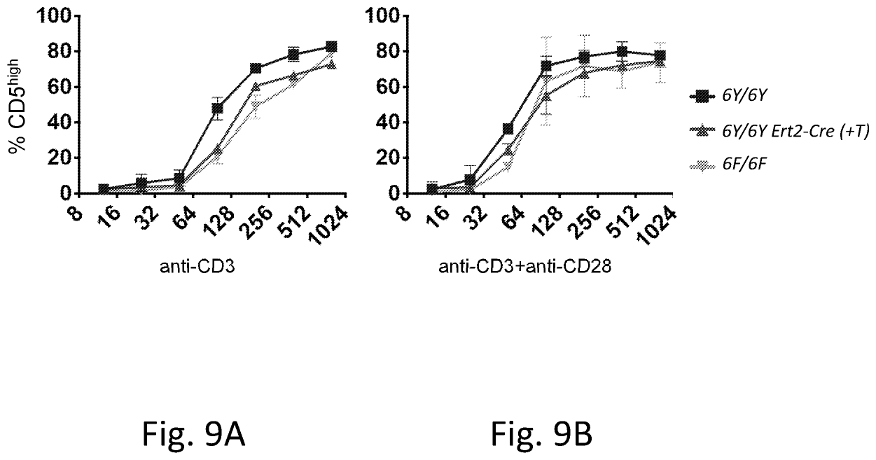

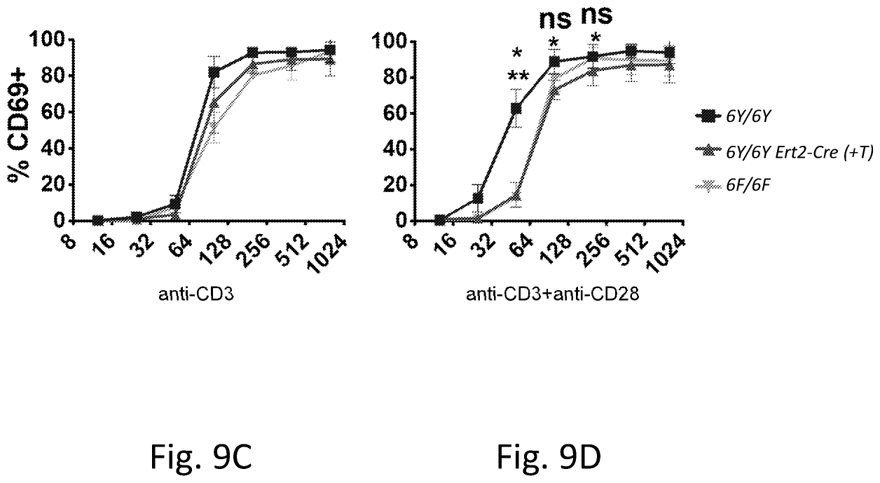

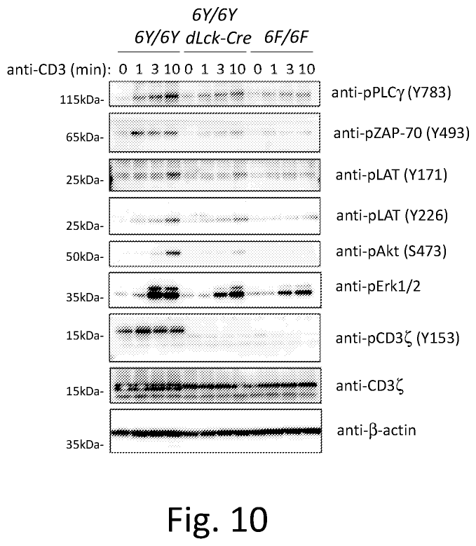

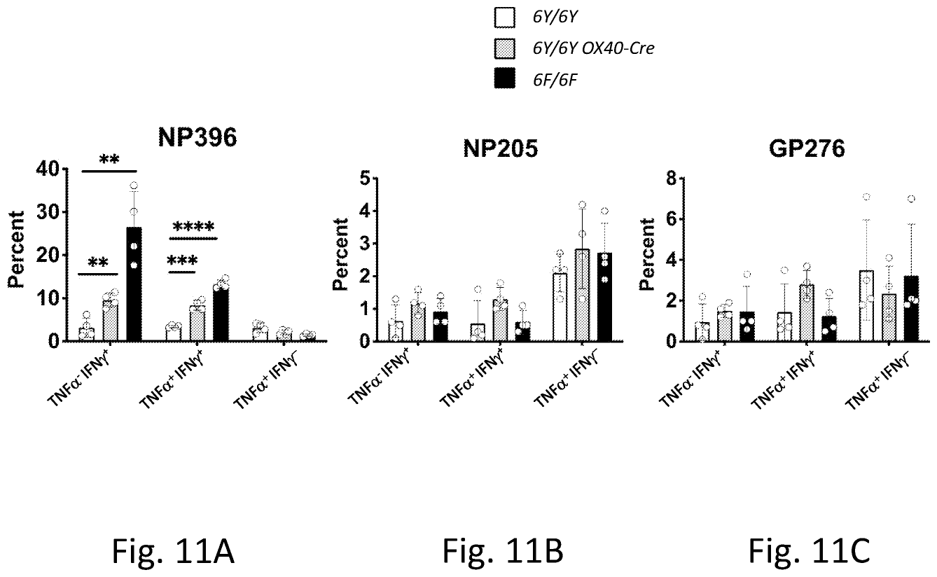

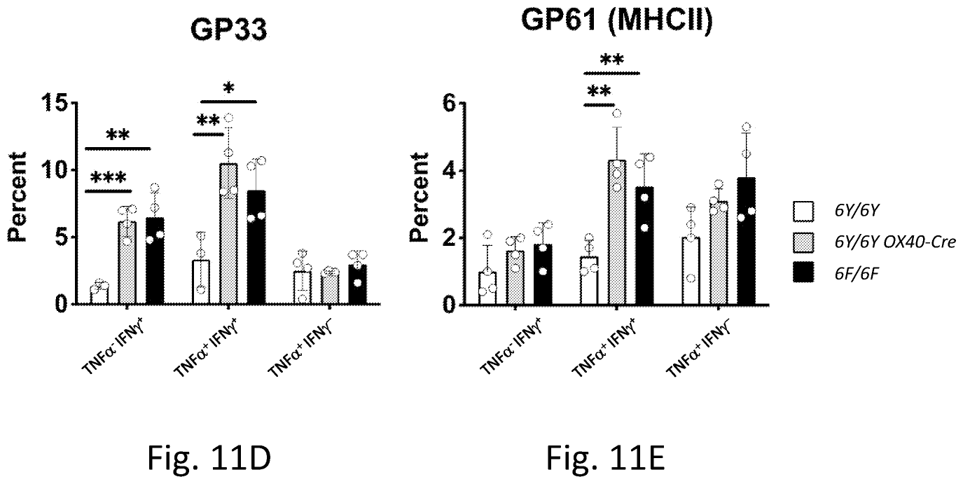

It was hypothesized that the relatively mild effect of germline CD3zeta ITAM inactivation on peripheral T cell functions observed with previous mouse models (Shores et al., J. Exp. Med., 185: 893-900 (1997); Pitcher et al., Eur. J. Immunol., 35: 3643-3654 (2005); Hwang et al., J. Exp. Med., 209: 1781-1795 (2012); Hwang et al., Nat. Commun., 6: 6982 (2015); Ardouin et al., Immunity, 10: 409-420 (1999)) could be due to compensatory changes in the TCR signaling response imposed during the process of thymocyte selection (Gaud et al., Nature Rev. Immunol., 18: 485-497 (2018)). To bypass these potential compensatory effects, a ‘knock-in’ experimental ‘CD3zeta-switch’ mouse model was generated. In this mouse model, a mutant CD3zeta chain [where all six of the ITAM tyrosines (Y) are replaced by a phenylalanine (F) rendering CD3zeta signaling defective] can be substituted for the wild-type (WT) CD3zeta chain by Cre-mediated recombination (FIG. 1A) (Hwang et al., J. Exp. Med., 209: 1781-1795 (2012)). ERT2-Cre CD3zeta 6Y/6Y mice were generated where the switch from WT Flag-tagged CD3zeta (6Y) to Myc-tagged CD3zeta (6F) chain could be induced by tamoxifen (Ventura et al., Nature, 445: 661-665 (2007)). dLCK-cre+ 6Y/6Y mice were generated where the Cre-mediated 6Y to 6F switch occurs after thymocyte selection (Zhang et al., J. Immunol., 174: 6725-6731, (2005)). Stimulation of tamoxifen treated Ert2-Cre+ 6Y/6Y CD8 T cells or CD8 T cells from dLCK-cre+ 6Y/6Y mice with anti-CD3 TCR cross-linking antibody alone or in combination with anti-CD28 antibody elicited responses that phenocopied germline 6F-CD3zeta expressing T cells (Hwang et al., J. Exp. Med., 209: 1781-1795 (2012); Hwang et al., Nat. Commun., 6: 6982 (2015)); that is, they exhibited similar or slightly lower activation and proliferation responses compared to control 6Y/6Y CD8 T cells (FIGS. 8A-8C and 9A-9D). Pre-selection germline 6F/6F T cells and post-selection 6Y- to 6F-CD3zeta ‘switched’ 6F-CD3zeta expressing T cells also displayed a similar mild to moderate reduction in the activation of proximal TCR signaling proteins, as assessed by phosphorylation, after stimulation with anti-CD3 antibody (FIG. 10). To evaluate TCR-mediated in vivo responses, age and gender-matched 6F/6F germline mice, OX40-Cre+ 6Y/6Y mice in which Cre is induced by TCR activation (Klinger et al., J. Immunol., 182: 4581-4589 (2009)), and control 6Y/6Y mice, were infected with LCMV Armstrong and antigen-specific T cell responses were evaluated 8 days after infection. Following in vitro peptide re-stimulation, examination of the percentage of LCMV peptide-specific IFNgamma⁺TNFalpha⁺ T-cells indicated that the response of 6F-CD3zeta expressing CD8 T cells to some (NP396, GP33) but not other (NP205, GP276) LCMV-specific T cell MHC-I restricted peptides, and the response of 6F-CD3zeta expressing CD4 T cells to the MHC-II restricted GP61 peptide was superior to that of control 6Y-CD3zeta expressing T cells regardless of whether expression of 6F-CD3zeta was germline encoded or induced in activated cells (FIGS. 11A-11E). Once again, the post-selection 6Y- to 6F-CD3zeta switched mouse model phenocopied the germline 6F/6F model; but unexpectedly, both showed superior ability to mount an immune response to several LCMV derived peptides compared to ‘WT’ 6Y/6Y mice. These results demonstrated that the relatively modest reduction in most TCR signaling responses caused by CD3zeta ITAM inactivation previously observed in germline 6F/6F mice cannot be explained by compensatory ‘TCR tuning’ during T cell selection. These results suggested that the effect of CD3zeta ITAM inactivation on TCR-mediated responses is variable, presumably depending upon the nature of the TCR/peptide-MHC interaction.

### Example 7

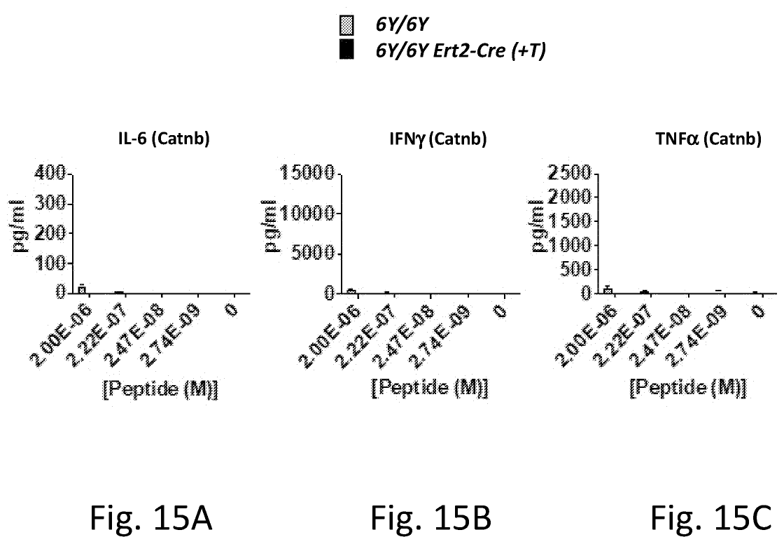

This example demonstrates that 6F-CD3z expressing T cells have a lower threshold of activation but are not overtly self-reactive (FIGS. 15-18).

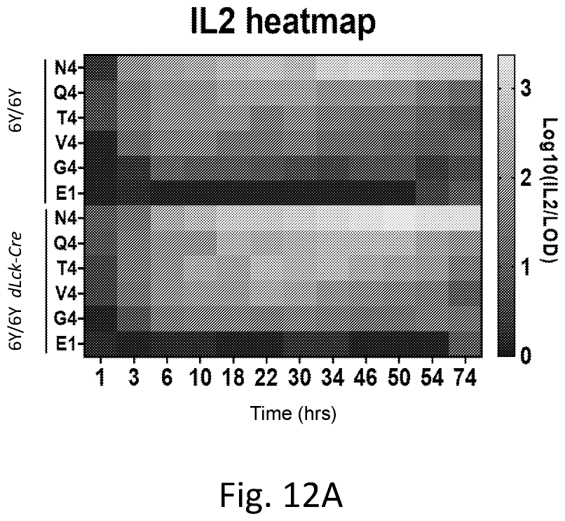

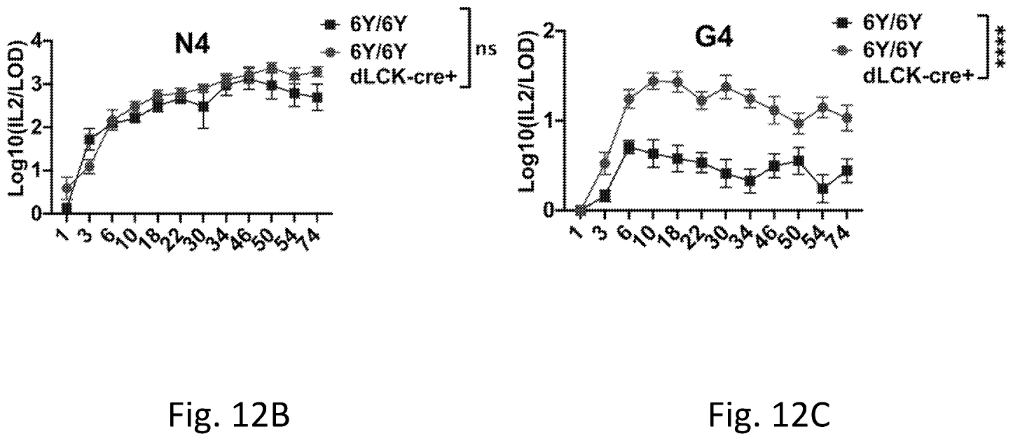

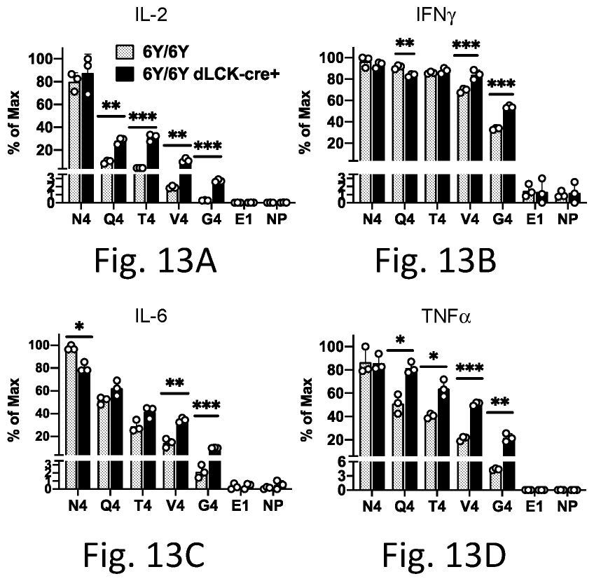

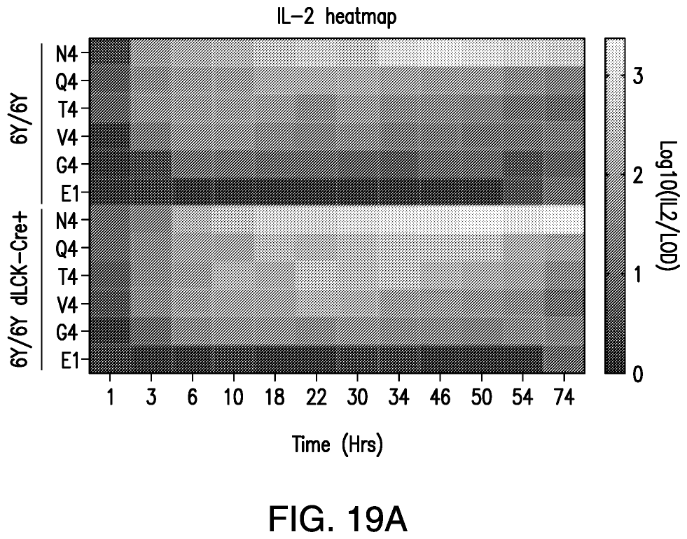

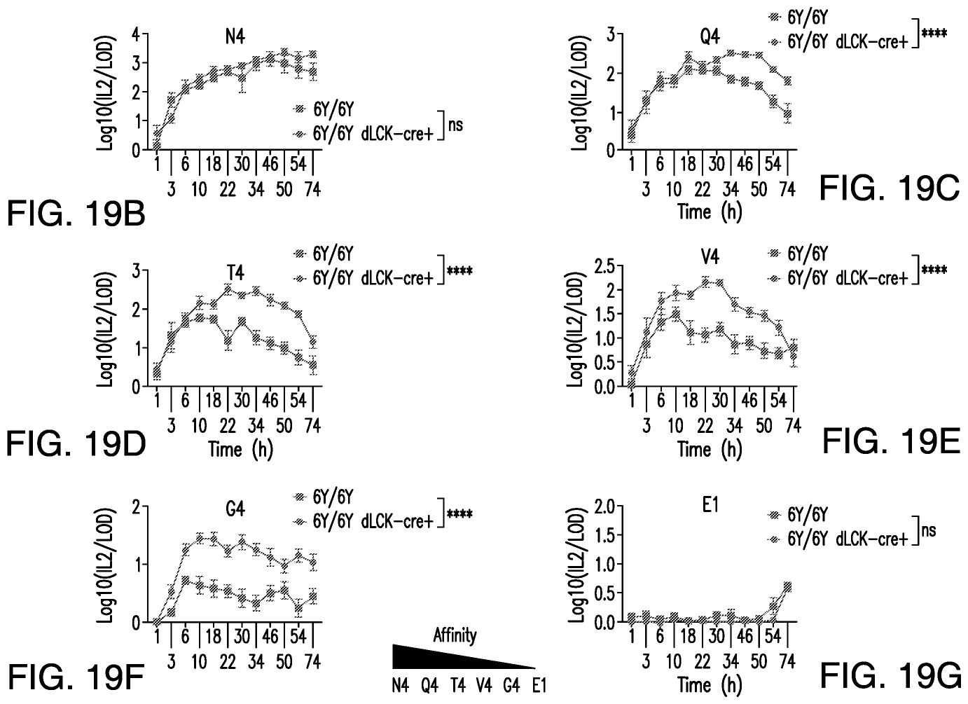

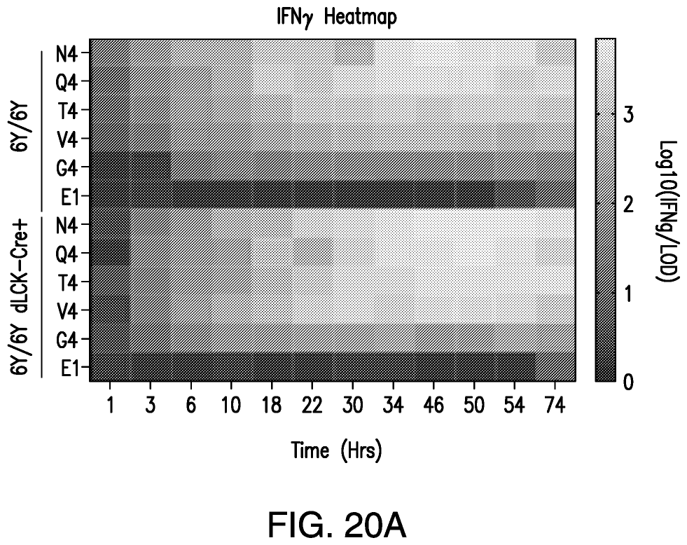

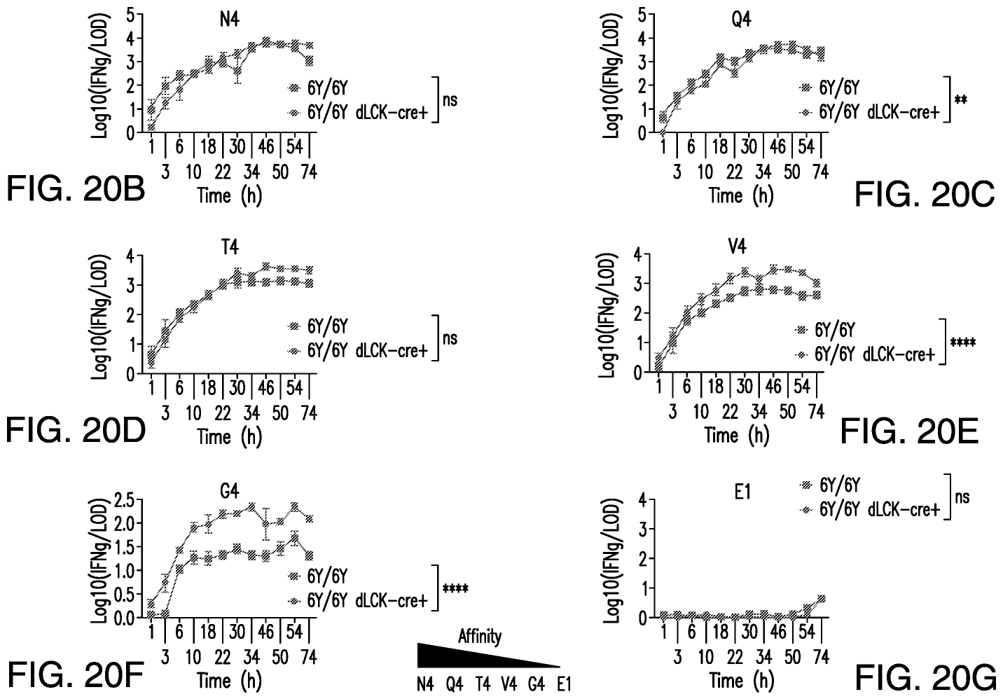

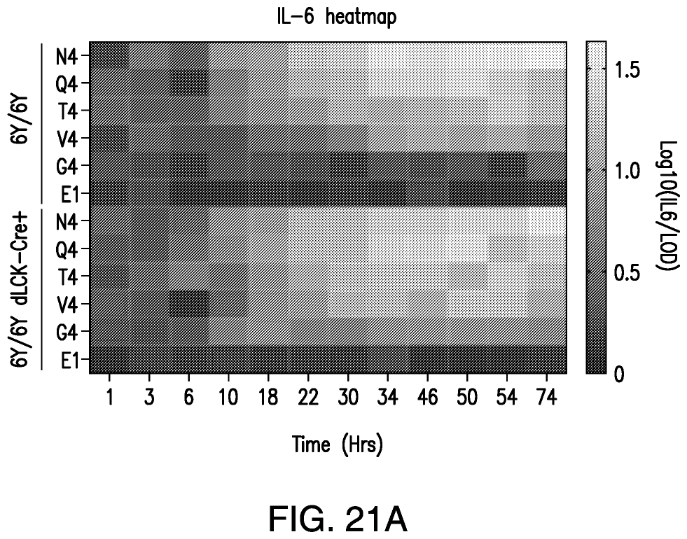

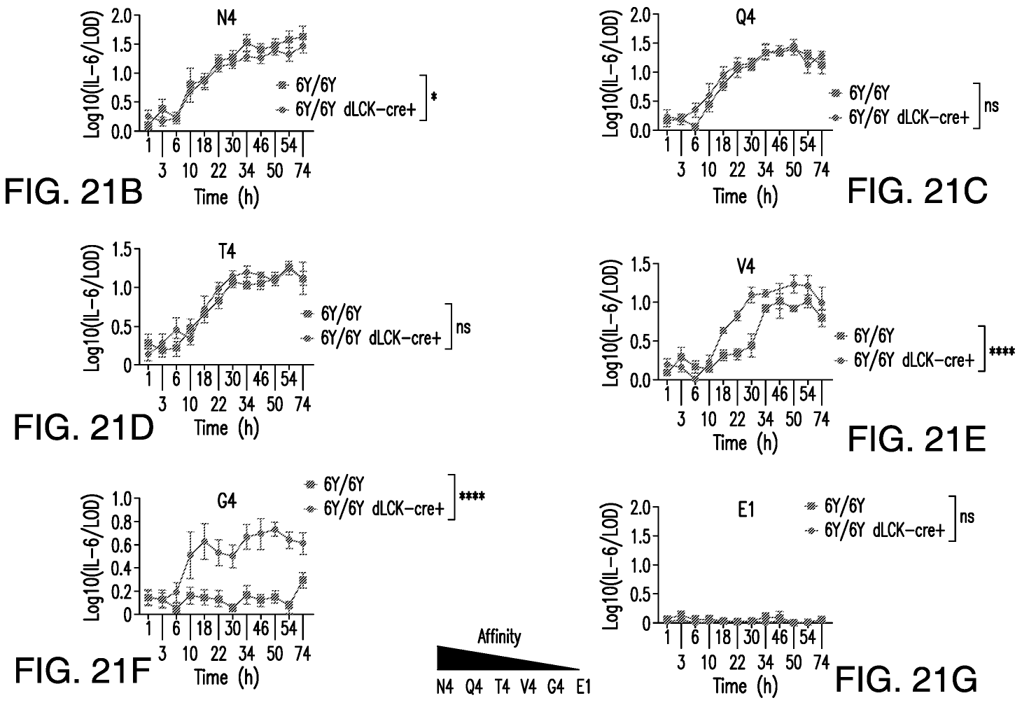

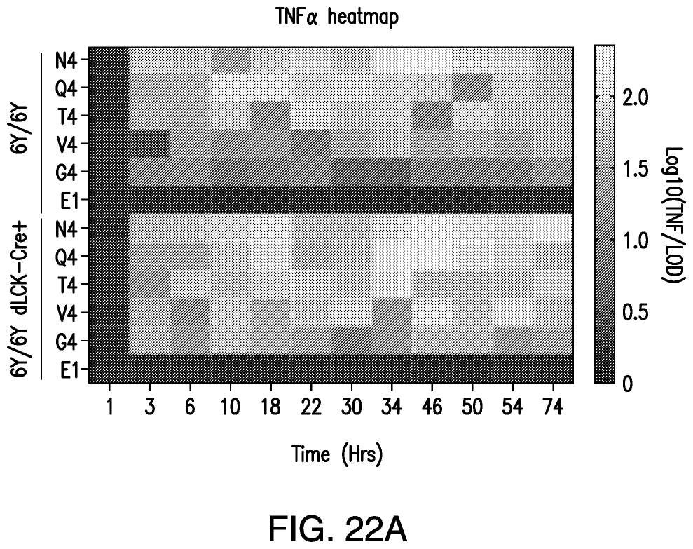

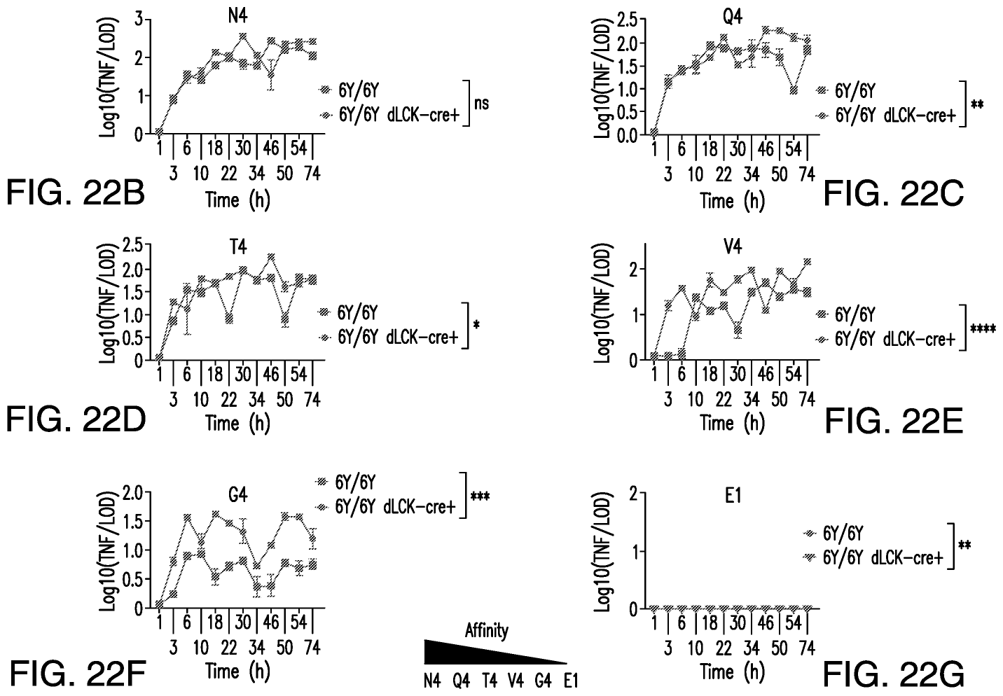

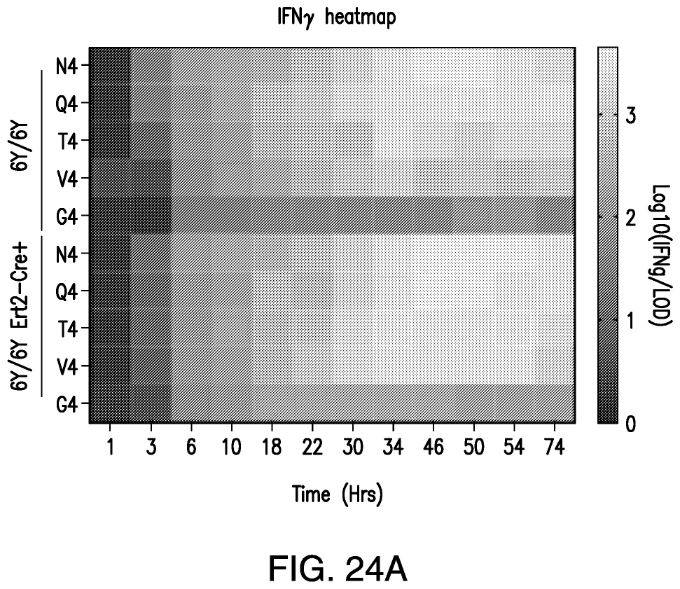

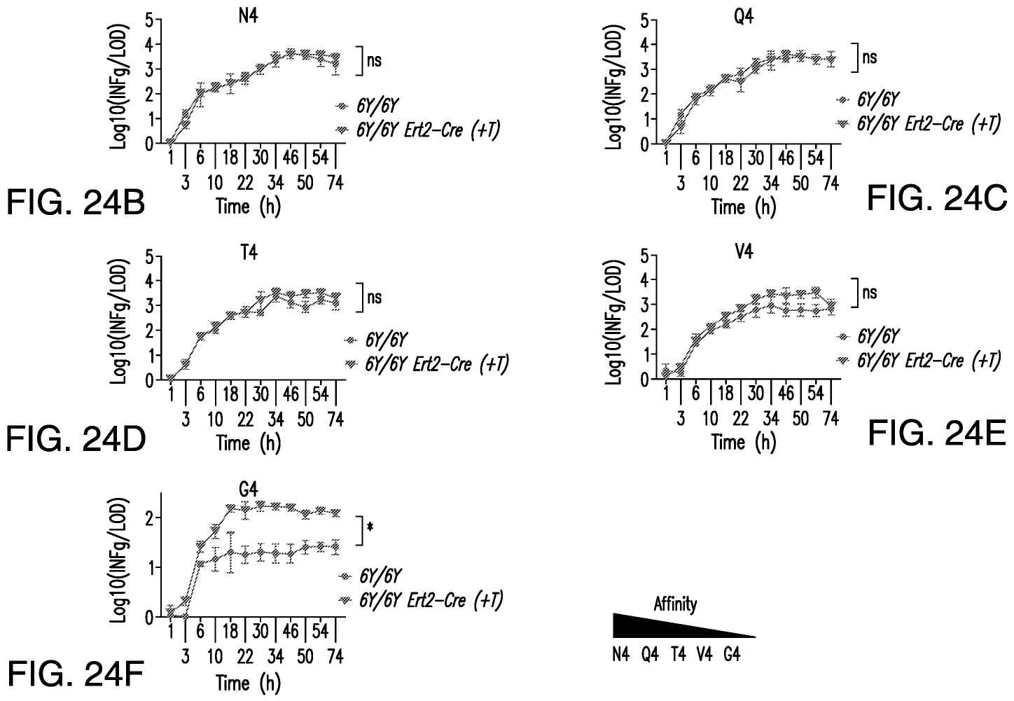

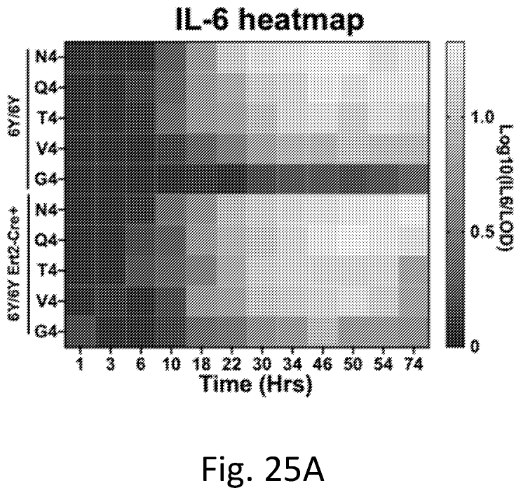

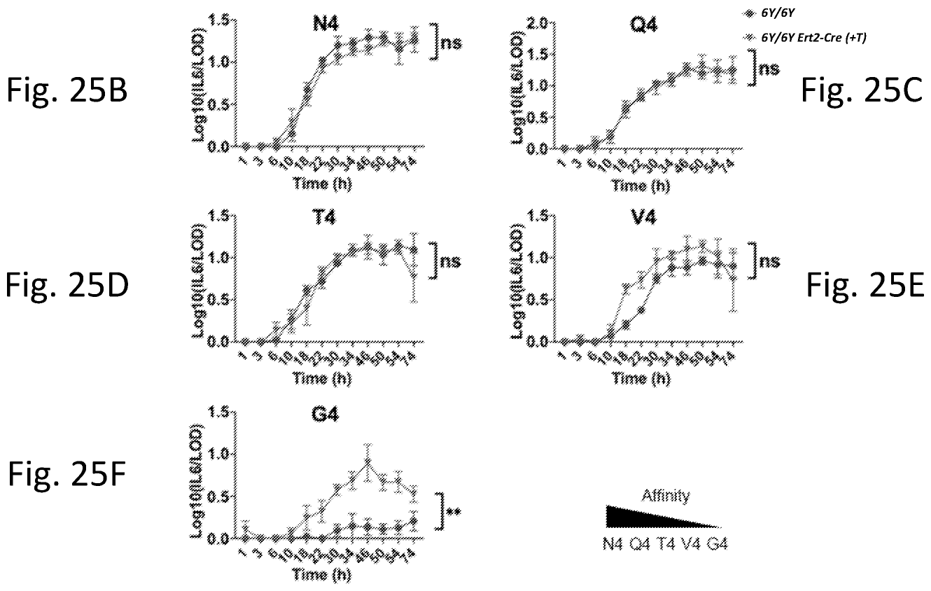

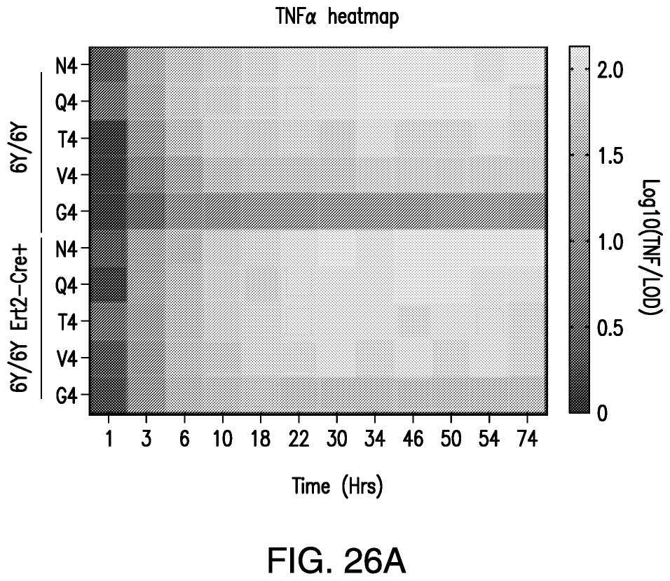

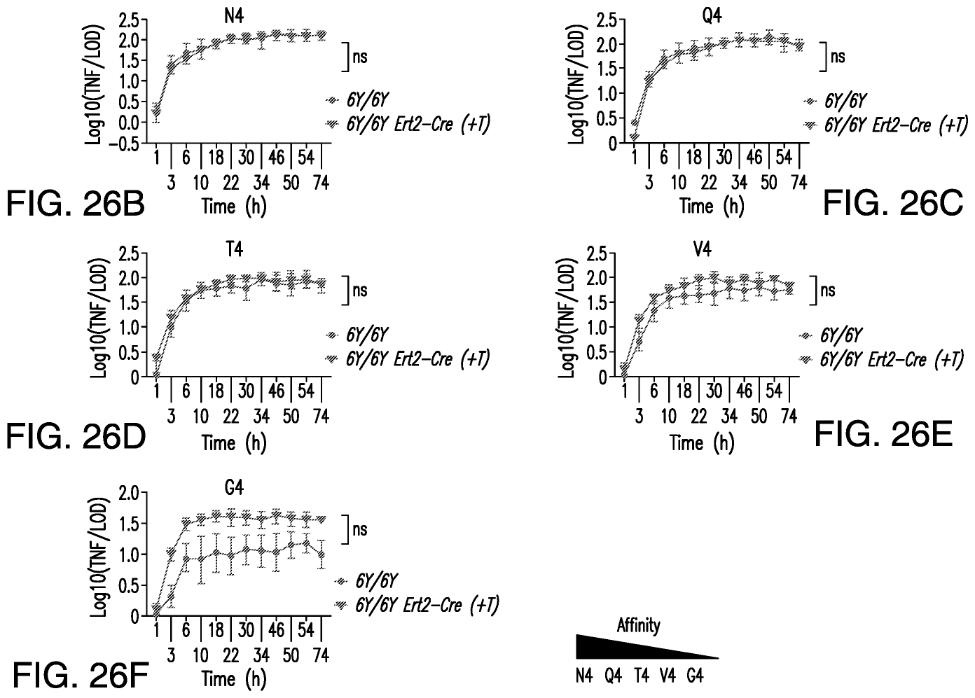

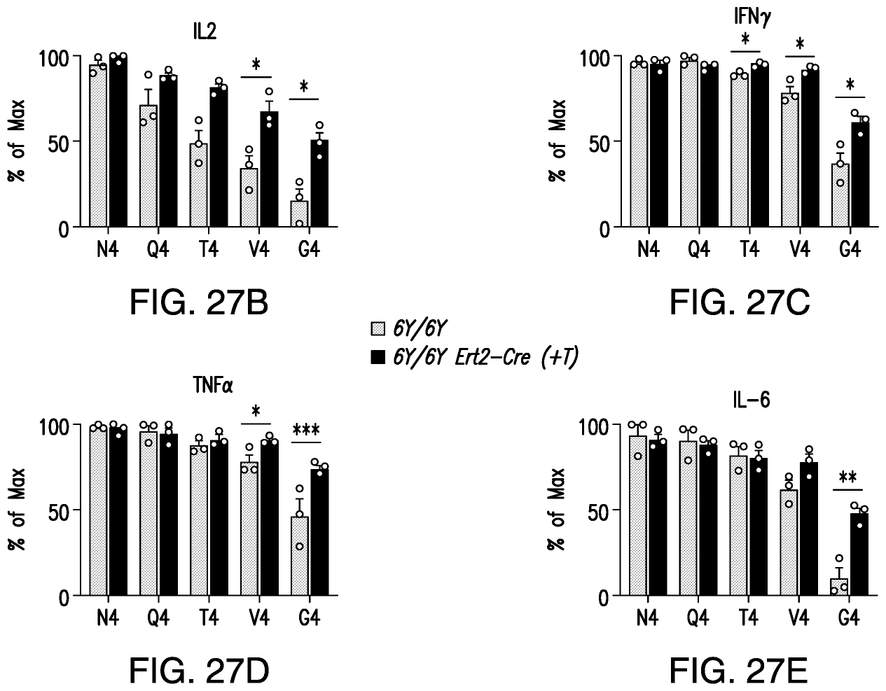

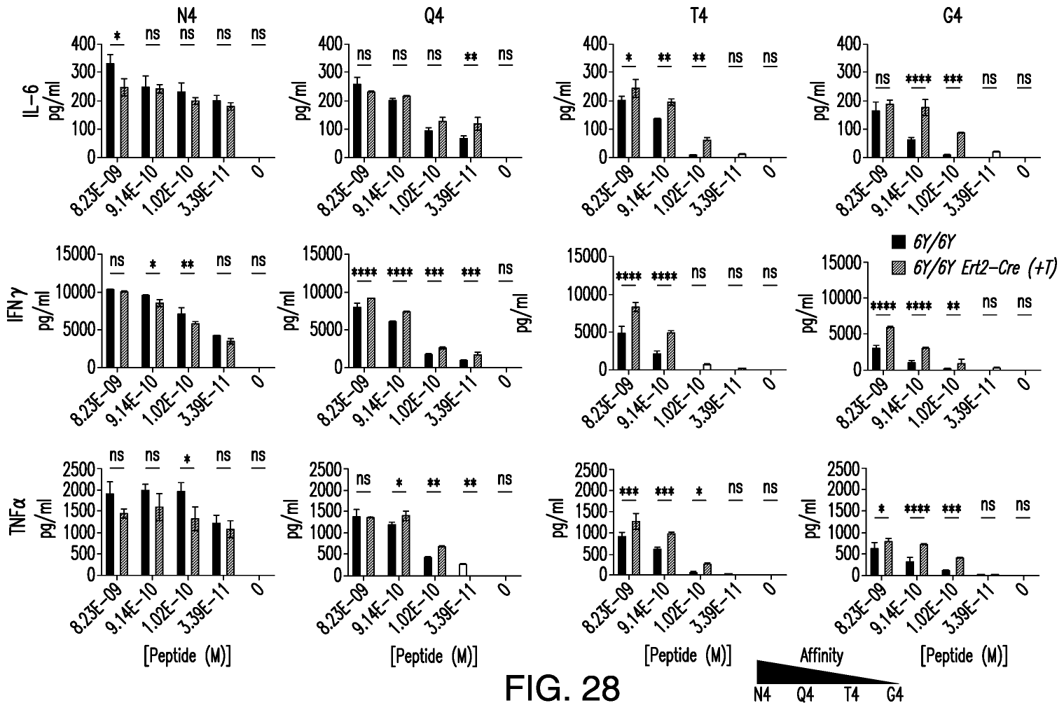

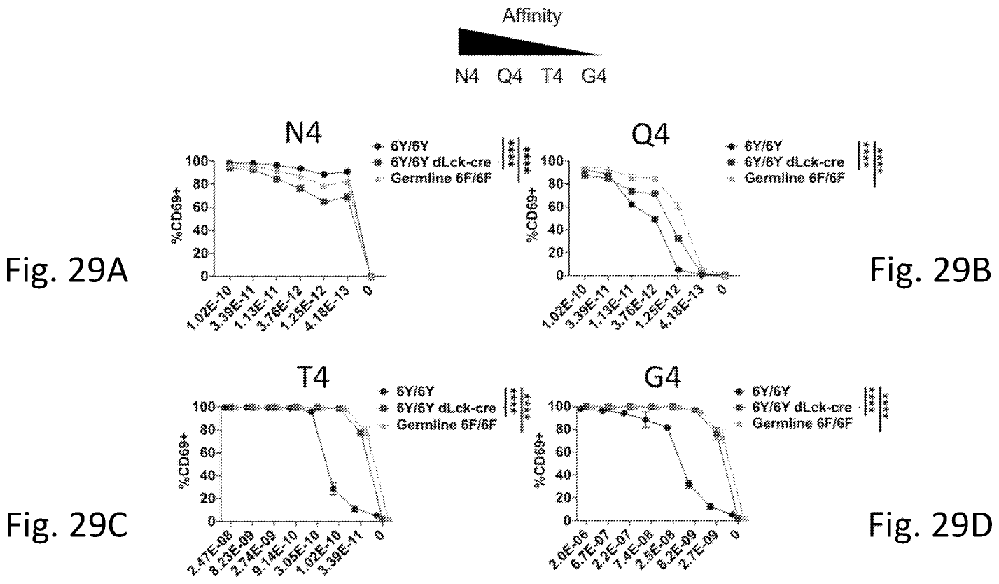

It was hypothesized that strong artificial stimulation induced by anti-CD3 mediated TCR cross-linking could be masking subtle signaling variations. Therefore, a more physiological system that utilizes the OTI-TCR transgenic model was used. OTI TCR transgenic T cells express a defined α/βTCR specific for a chicken ovalbumin derived peptide (OVA/N4) (Hogquist et al., Cell, 76: 17-27 (1994); Daniels et al., Nature, 444: 724-729 (2006)). A TECAN multiplexing automated platform was used to monitor cytokine expression over time by OTI CD8 T cells stimulated in vitro with Antigen Presenting Cells (APCs) pulsed with agonist peptide OVA/N4 or the OVA/N4-derived Altered Peptide Ligands (APLs) Q4, T4, V4 and G4, listed in decreasing order of affinity for the OT1 TCR covering a range of ligand potency/functional avidity of >7,000-fold (Daniels et al., Nature, 444: 724-729 (2006); Zehn et al., Nature, 458: 211-214 (2009)) (Table 5). Under these conditions, 6Y/6Y dLCK-cre+ OTI CD8 T cells which expressed 6F-CD3ζ produced more IL-2 when stimulated with the lower affinity peptides Q4, T4, V4, and G4 compared to 6Y/6Y OTI CD8 T cells, whereas neither T cell genotype reacted to the antagonist peptide, E1 (Table 5, FIGS. 12A-12C and FIGS. 19A-19G). A similar overall trend was observed with all four tested cytokines (IL-2, IL-6, IFNγ and TNFα) (FIGS. 13A-13D), although the sensitivity varied for each APL and cytokine revealing different TCR signal strength requirements for individual cytokine responses (FIGS. 19A-19G, FIGS. 20A-20G, FIGS. 21A-21G, and FIGS. 22A-22G). This phenotype was not specific to dLCK-Cre+ 6Y/6Y T cells since similar results were obtained with germline 6F/6F T cells and with 6Y/6Y OTI Ert2-Cre+ T cells where the CD3ζ 6Y- to 6F-CD3ζ switch was induced in vitro in purified OTI CD8 T cells by 4-OH tamoxifen immediately prior to TCR stimulation (FIGS. 23A-23F, FIGS. 24A-24F, FIGS. 25A-25F, FIGS. 26A-26F, and FIGS. 27A-27D). Means of cytokine concentrations over time revealed that control 6Y/6Y (dLCK-Cre−) OTI T cells produced more IL-6 and IFNγ compared to 6F-CD3ζ expressing 6Y/6Y dLCK-Cre+ OTI T cells when challenged with high affinity peptides (N4 and Q4) in line with the previous observation with anti-CD3 antibody cross-linking, suggesting a dual, antinomic role of CD3ζ ITAMs depending on the affinity of the TCR/ligand interaction. Considering that most previous studies examining the role of CD3ζ in TCR signaling found either no impact or a negative impact of CD3ζ ITAM inactivation on TCR signaling (Hwang et al., J. Exp. Med., 209: 1781-1795 (2012); Hwang et al., Nat. Commun., 6: 6982 (2015); Guy et al., Nat. Immunol., 14: 262-270 (2013)), whether these data could be reconciled with those results was investigated by varying both affinity and avidity in the CD3ζ 6Y to 6F switch OTI model system. It was found that the differences in the cytokine response of OTI 6Y/6Y and OTI 6F/6F CD8 T cells to high or low affinity ligands were markedly diminished when the concentration of ligand, and consequently the avidity of the TCR-pMHC interaction, was increased (FIG. 28). A similar pattern was observed for TCR signaling-induced expression of the surface activation marker CD69 (FIGS. 29A-29D).

Table 5 shows the ligand potency of the different OTI peptides relative to N4 (Surh et al., Immunity, 29: 848-862 (2008)).

TABLE 5

| SEQ ID NO: | OVA Peptide | Ligand potency |
|---|---|---|
| 26 | N4: SIINFEKL | 1 |
| 27 | A2: SAINFEKL | 2.7 |
| 28 | Y3: SIYNFEKL | 4.3 |
| 29 | Q4: SIIQFEKL | 18-39 |
| 30 | T4: SIITFEKL | 71-122 |
| 31 | V4: SIIVFEKL | 680 |
| 32 | G4: SIIGFEKL | 7515 |
| 33 | E1: EIINFEKL | 56524 |

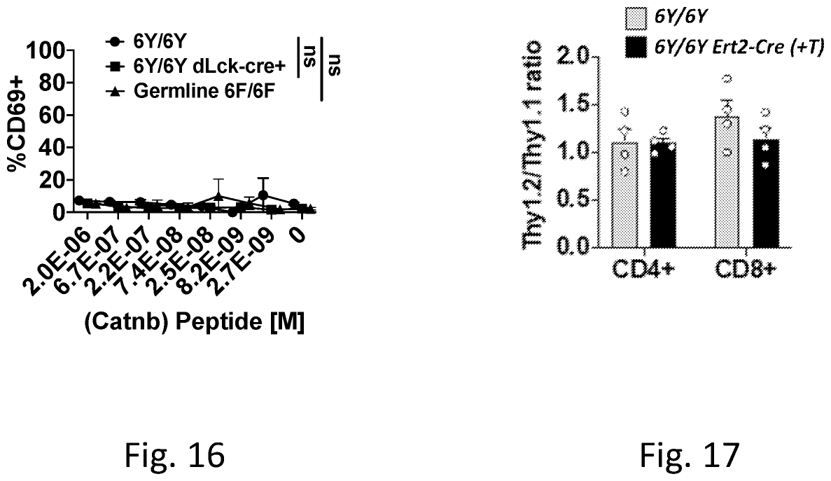

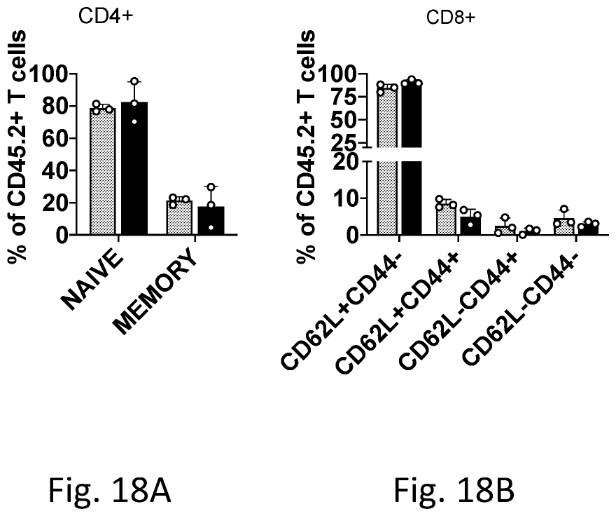

OTI 6F/6F CD8 T cells (derived from tamoxifen treated OTI 6Y/6Y Ert2-cre+ CD8 T cells) proliferated more than WT (CD3z^{-/-}) OTI CD8 cells in vivo in response to low affinity Q4 or T4 APL-peptide pulsed APCs, but not in response to high affinity N4 peptide pulsed APCs (FIGS. 14A-14E). Notably, 6F-CD3z expressing OTI CD8 T cells did not respond to the OTI self-peptide, catnb (Santori et al., Immunity, 17: 131-142 (2002)) indicating that 6F-CD3z does not render T cells sensitive to activation by self-ligands (FIGS. 15A-15C and FIG. 16). In lymphopenic mice, proliferation of naïve T cells is driven by TCR-self-pMHC interactions together with cytokine signals through the IL-7 receptor (Surh et al., Immunity, 29: 848-862 (2008)). The affinity of the TCR toward self-pMHC determines the extent of naive T cell lymphopenia-induced proliferation (Kassiotis et al., J. Exp. Med., 197: 1007-1016 (2003); Kieper et al., J. Immunol., 172: 40-44 (2004)) and can be enhanced by genetic alterations that increase the TCR signaling response to low affinity peptides (Salmond et al., Nat. Immunol., 15: 875-883 (2014)). Purified 6Y/6Y T cells and 6F/6F T cells generated from tamoxifen treated Ert2-cre+ 6Y/6Y T cells proliferated to the same extent when co-injected into T/B cell deficient Rag2^{-/-} hosts (FIG. 17). Moreover, in irradiated mice reconstituted with a 1:1 mixture of bone marrow from CD45. 1 B6 and CD45.2 6Y/6Y Ert2-cre+ or Ert2-Cre+ (CD3z^{+/+}) mice, the ratio and naïve/memory surface phenotype of CD4 and CD8 T cells containing 6Y-CD3z or 6F-CD3z was unchanged two weeks after tamoxifen gavage (FIGS. 18A-18B). Collectively, these findings indicated that expression of 6F-CD3z does not result in overt self-reactivity consistent with the absence of autoimmune disease in either germline 6F/6F or dLck-Cre+ 6Y/6Y mice (Hwang et al., J. Exp. Med., 209: 1781-1795 (2012)).

### Example 8

This example demonstrates that 6F-CD3z expressing T cells display enhanced TCR signaling responses when stimulated with low affinity antigens.

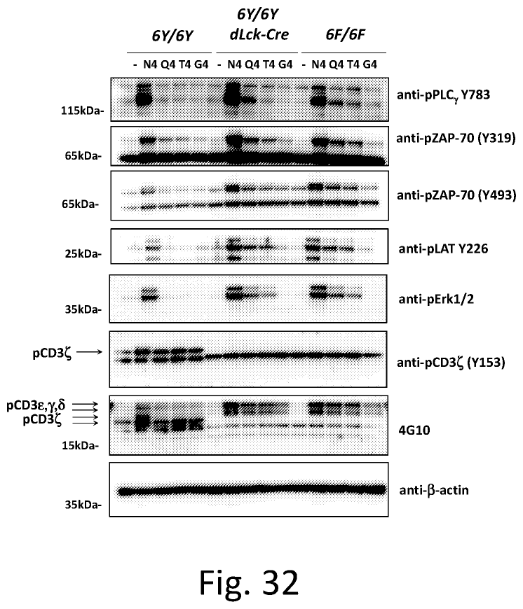

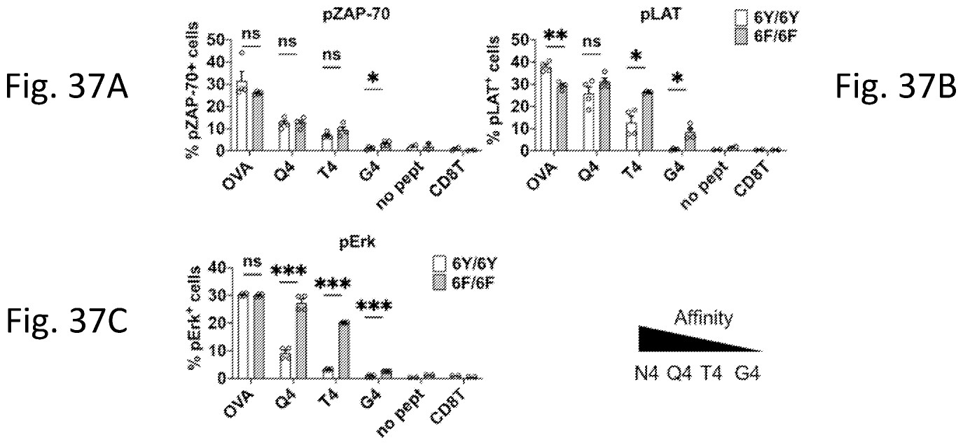

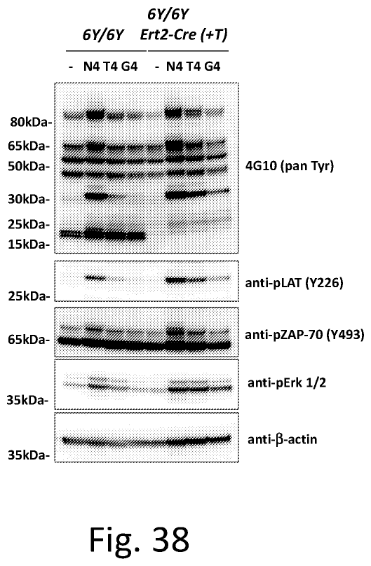

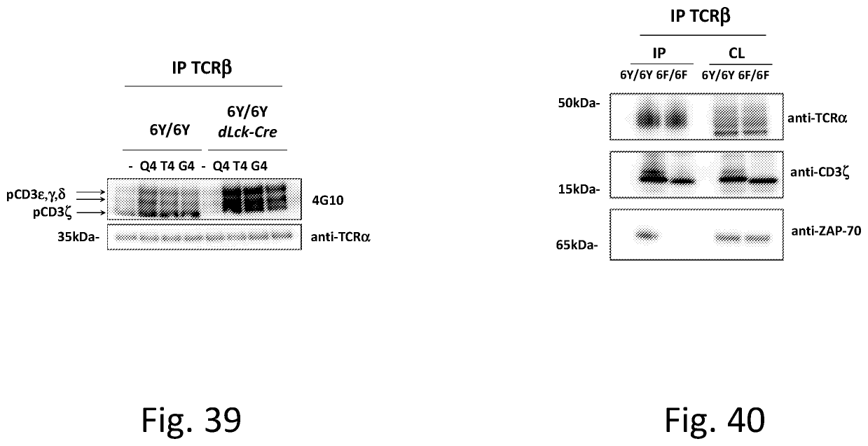

It was next checked if TCR signaling responses were affected by 6F-CD3ζ. The expression of IRF4 has previously been shown to reflect TCR signal intensity (Iwata et al., Nat. Immunol., 18: 563-572 (2017)). 6Y/6Y Ert2-cre+ OTI CD8 T cells induced to express 6F-CD3ζ in vitro and then stimulated with N4 peptide pulsed APCs for 48 h showed a slightly lower expression of IRF4 than 6Y/6Y (Ert2-cre) OTI CD8 T cells but expressed significantly more IRF4 than 6Y/6Y OTI CD8 T cells when stimulated with the lower affinity G4 peptide (FIG. 30). It was then checked if proximal TCR signaling was affected by 6F-CD3ζ by performing phos-flow and phospho-protein western blotting. Phosphorylation of the TCR-proximal signaling proteins ZAP-70 (Y319), LAT (Y171) and Erk (T202/Y204) was significantly increased in 6F-CD3ζ expressing CD8 T cells compared to 6Y-CD3ζ expressing CD8 T cells when stimulated with APC pulsed with the low affinity peptides Q4, T4 and G4, but was similar or decreased compared to 6Y-CD3ζ expressing CD8 T cells when stimulated with the high affinity peptide N4 (FIGS. 31 and 37A-C). Similar results were obtained with OTI CD8 T cells stimulated with peptide-tetramers and analyzed by western blot for induced phosphorylation of LAT (pY226), ZAP-70 (pY319 and pY493), PLC-71 (pY783), and pErk1/2 (FIG. 32). These results were confirmed in OTI 6Y/6Y ERT2Cre+ tamoxifen-induced in vitro-switched CD8 T cells (FIG. 38). Calcium flux responses were significantly enhanced in dLck-Cre+ 6Y/6Y CD8 T cells (which expressed 6F-CD3ζ) following stimulation with all OVA-derived peptides including the highest affinity N4 agonist peptide. As expected, CD3ζ phosphorylation was detected in 6Y-CD3ζ expressing CD8 T cells but not in 6F-CD3ζ expressing CD8 T cells. However, tyrosine phosphorylation of CD3-γ, -δ, -ε was markedly and specifically increased in 6F-CD3ζ expressing CD8 T cells after stimulation with N4, Q4, T4 and G4 peptide-tetramers (FIG. 32).

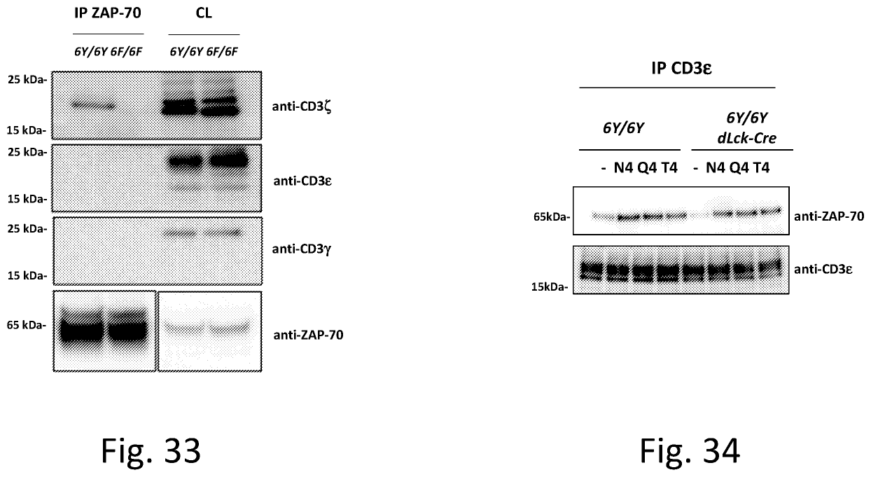

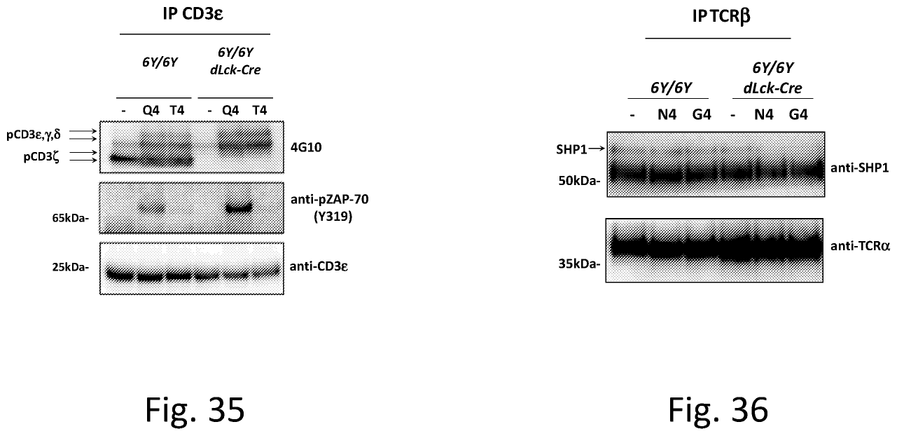

Phosphorylation of the CD3 ITAMs by the tyrosine kinase Lck is one of the first steps of TCR signaling and results in the recruitment of ZAP-70 kinase to the TCR through its specific interaction with dual tyrosine phosphorylated CD3 ITAMs and its subsequent activation (tyrosine phosphorylation) by Lck (Hatada et al., Nature 377: 32-38 (1995)). ZAP-70 is bound to phosphorylated CD3ζ ITAMs in un-activated ‘resting’ T cells and this has been speculated to represent a source of ‘primed’ enzyme. However, the pool of TCR-associated ZAP-70 has been shown to be catalytically inactive (van Oers et al., Immunity, 1: 675-685 (1994); Stefanova et al., Nature, 420: 429-434 (2002)). It was speculated that the increased phosphorylation of the CD3-γ, -δ and -ε subunits detected in activated T cells expressing 6F-CD3ζ could reflect an increase in the percentage of newly dual phosphorylated TCR ITAMs resulting in increased ZAP-70 recruitment to the TCR. It was confirmed that CD3-γ, -δ and -ε ITAMs were more strongly tyrosine phosphorylated in TCR immunoprecipitates from OTI dLck-Cre+ 6Y/6Y compared to OTI 6Y/6Y CD8 T cells (FIG. 33). Notably, ZAP-70 was not detected in TCR immunoprecipitates from unstimulated OTI dLck-Cre+ 6Y/6Y CD8 T cells (that expressed 6F-CD3ζ) in contrast to control, OTI 6Y/6Y CD8 T cells (that expressed 6Y-CD3ζ) (FIGS. 33,34,40). However, TCRs that contained 6F-CD3ζ recruited more ZAP-70 following stimulation with tetramers containing both low and high affinity peptides (FIG. 40) and were associated with more catalytically activate (Y319 phosphorylated) ZAP-70 (FIG. 35).

Discrimination between low and high affinity ligands has been proposed to be due to recruitment to the TCR of negative signaling regulators, one being SHP-1, a tyrosine phosphatase that directly dephosphorylates ZAP-70, CD3 ITAMs and Lck, although the mechanism of its recruitment to the TCR is still under debate (Stefanova et al., Nat. Immunol., 4: (2003); Plas et al., Science 272: 1173-1176 (1996)). SHP-1 binds mono-phosphorylated ITAMs within the signaling proteins FcRγ, DAP12 and Igα, which mediate receptor signaling in innate lymphocytes and B lymphocytes (Blank et al., Immunol. Rev., 232: 59-71 (2009)). Examination of SHP-1 recruitment to the TCR revealed that in resting T cells, SHP-1 was selectively bound to TCRs that contained 6F-CD3ζ, but SHP-1 was not detected in TCR immunoprecipitates from T cells that expressed 6F-CD3ζ after TCR engagement by either low or high affinity peptide-tetramers (FIG. 36). In contrast, SHP1 was not associated with TCRs in resting T cells that contain 6Y-CD3ζ but was recruited to the TCR in 6Y-CD3ζ expressing T cells following stimulation with either high or low affinity peptide-tetramers (FIG. 36) suggesting that SHP1 selectively inhibits TCRs that contain 6Y-CD3ζ following TCR engagement.

### Example 9

This example demonstrates the enhanced cytotoxicity of 6F-CD3z-expressing CTL against tumor cells bearing low affinity ligands.

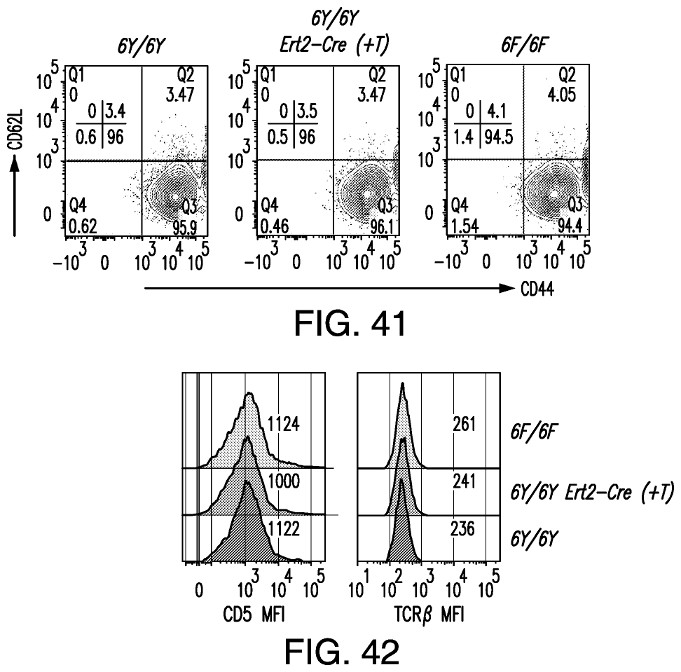

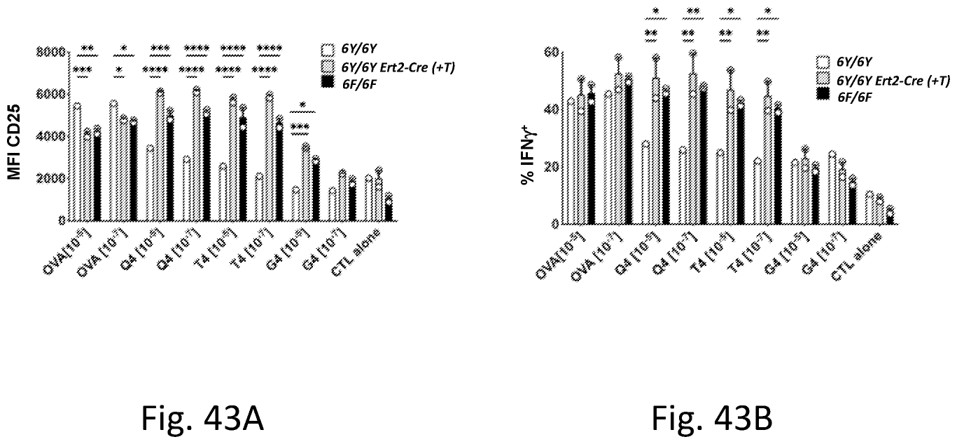

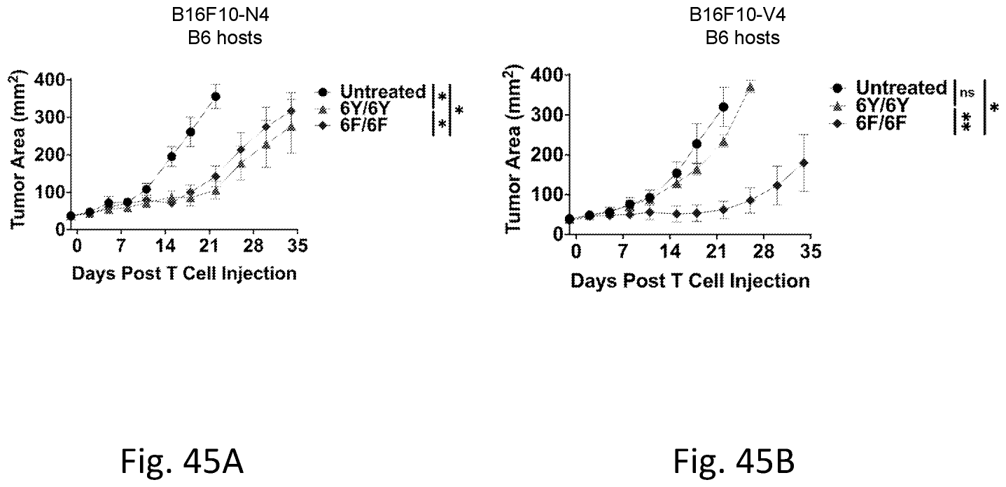

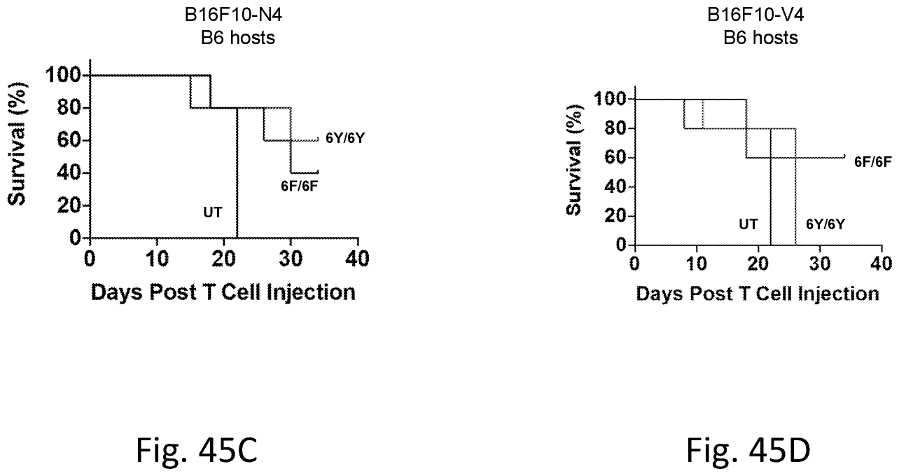

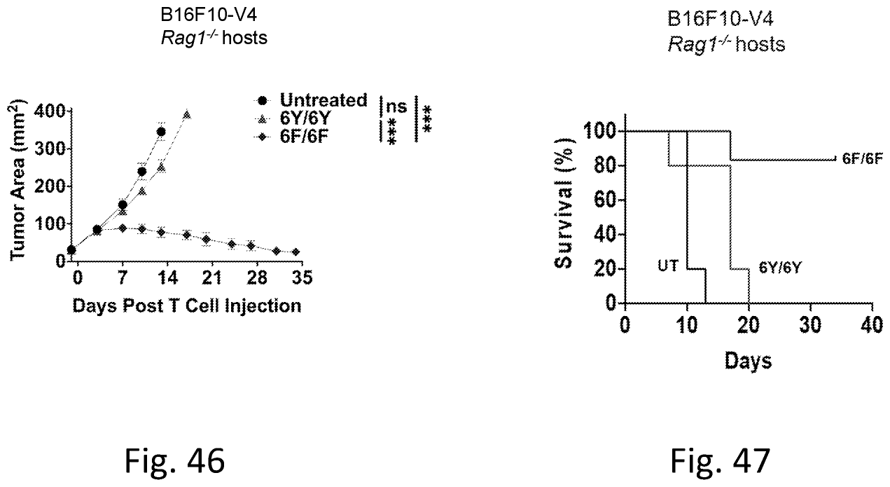

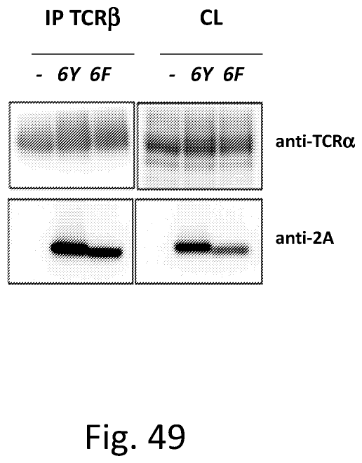

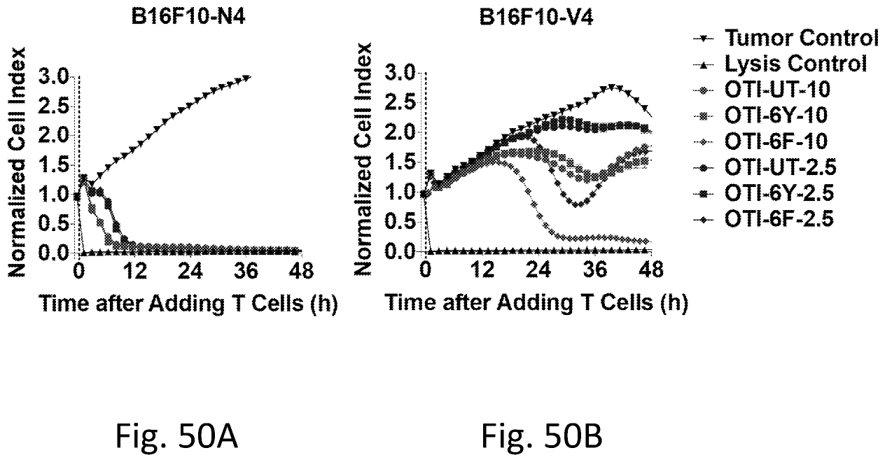

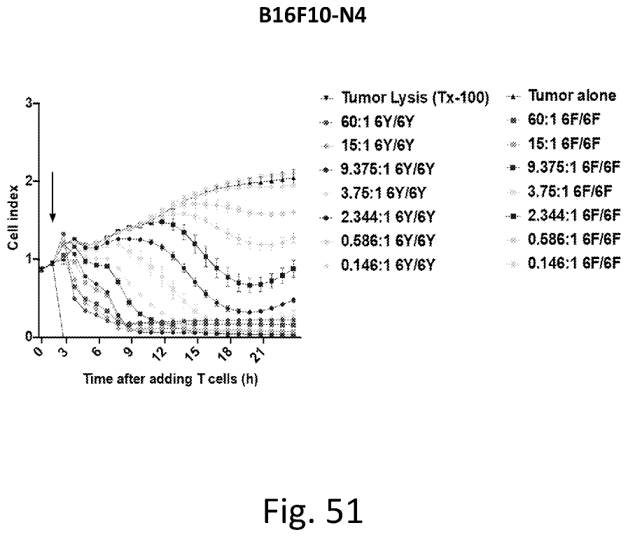

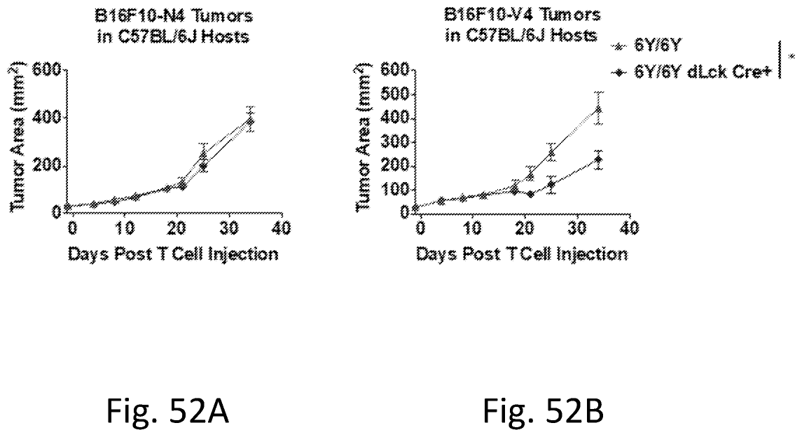

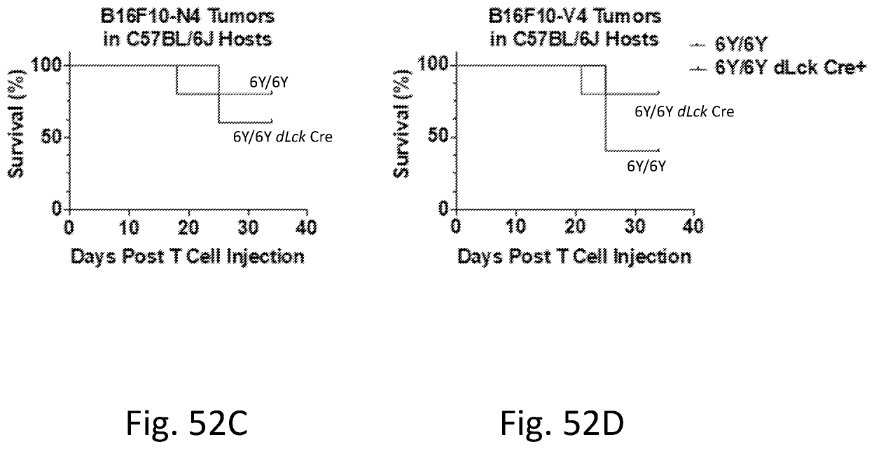

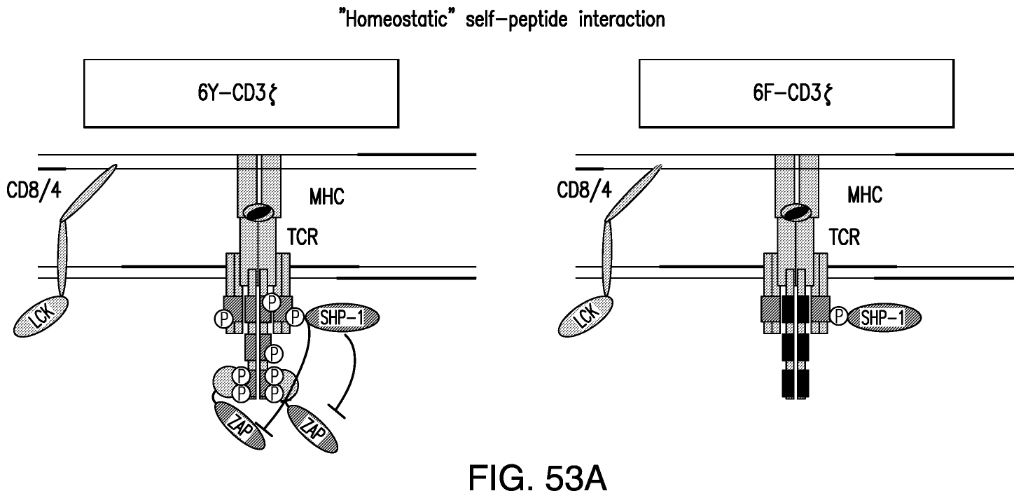

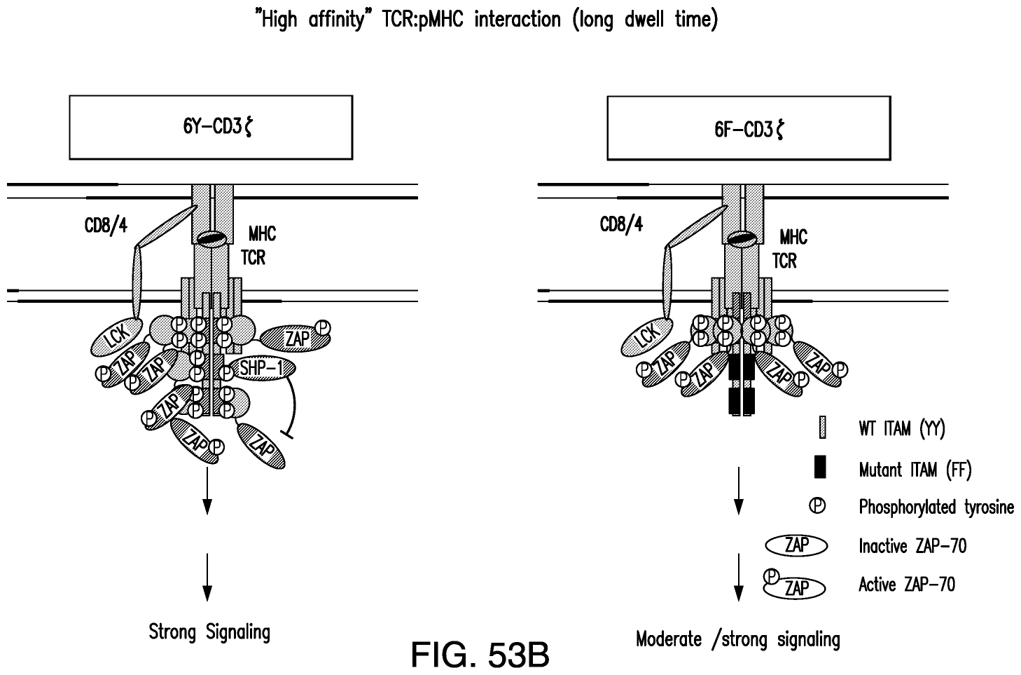

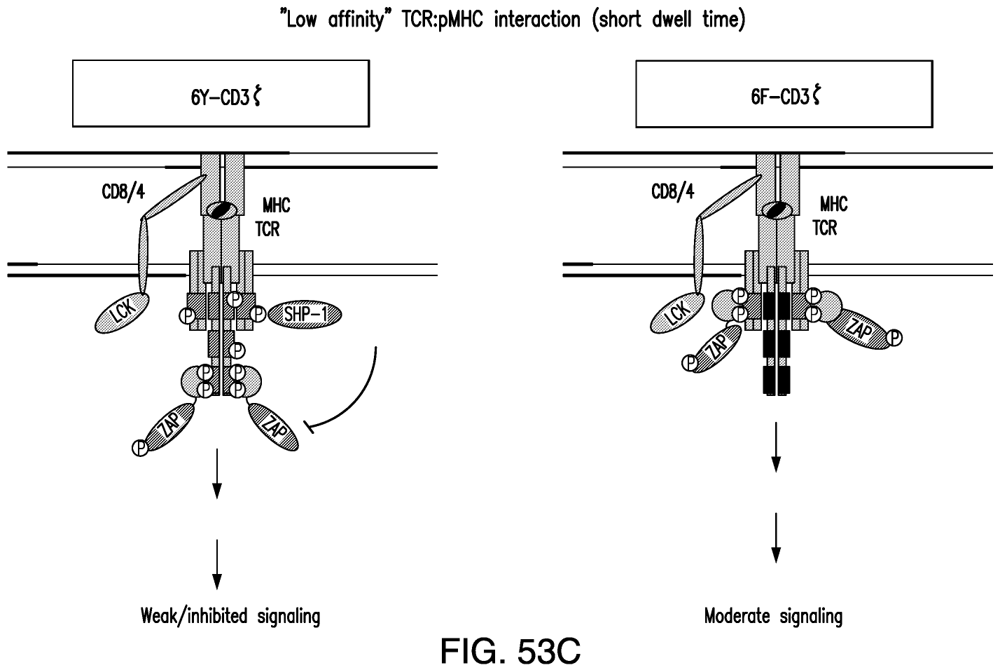

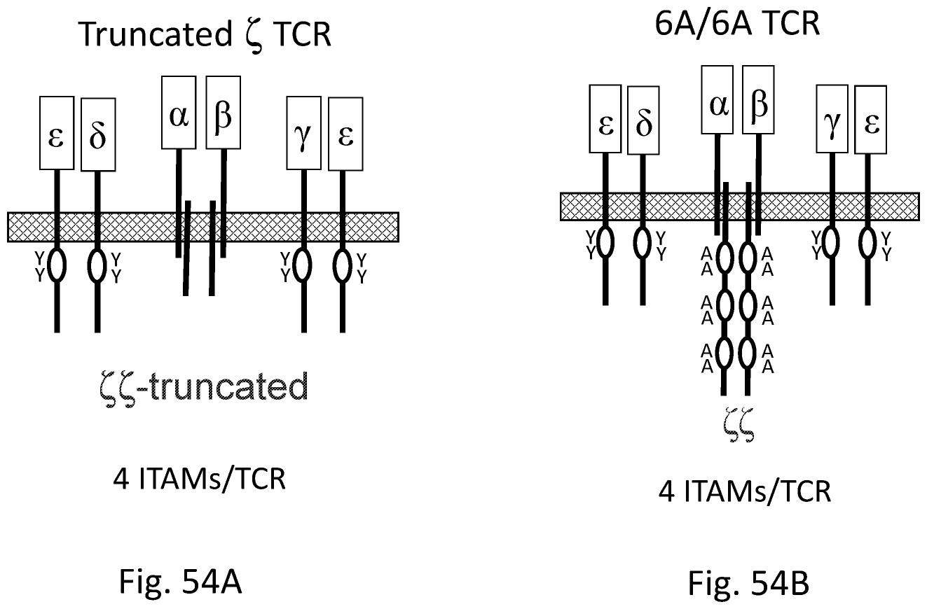

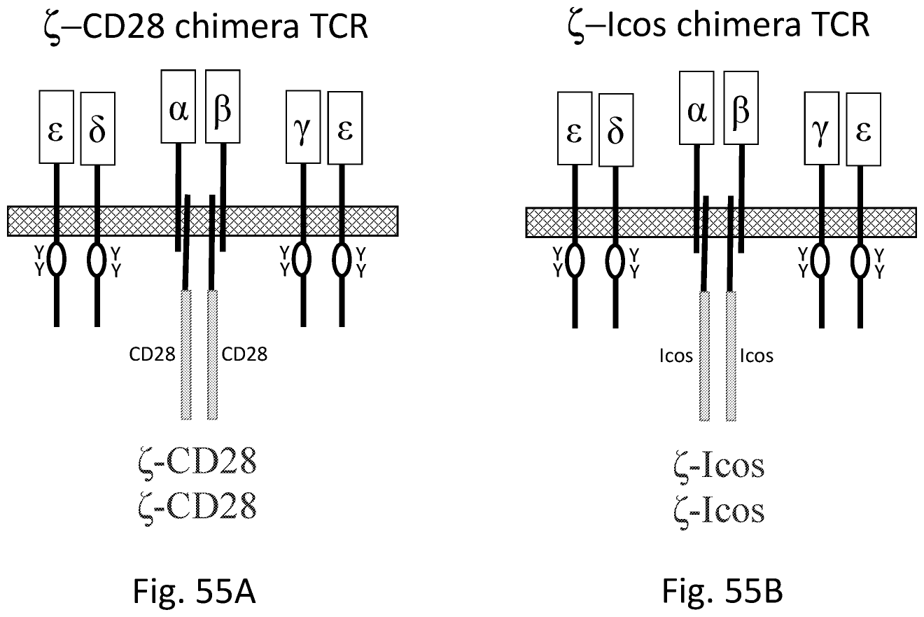

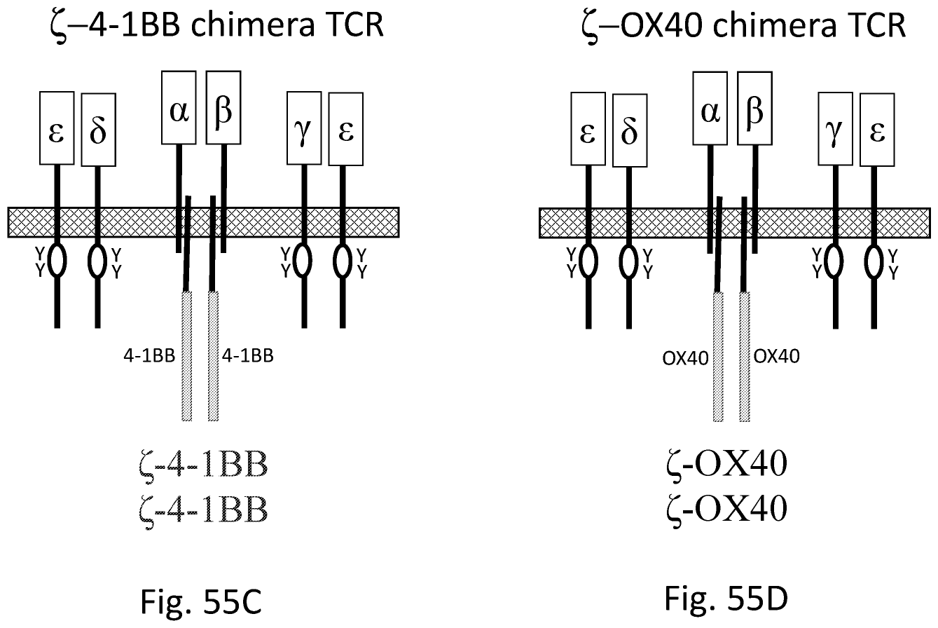

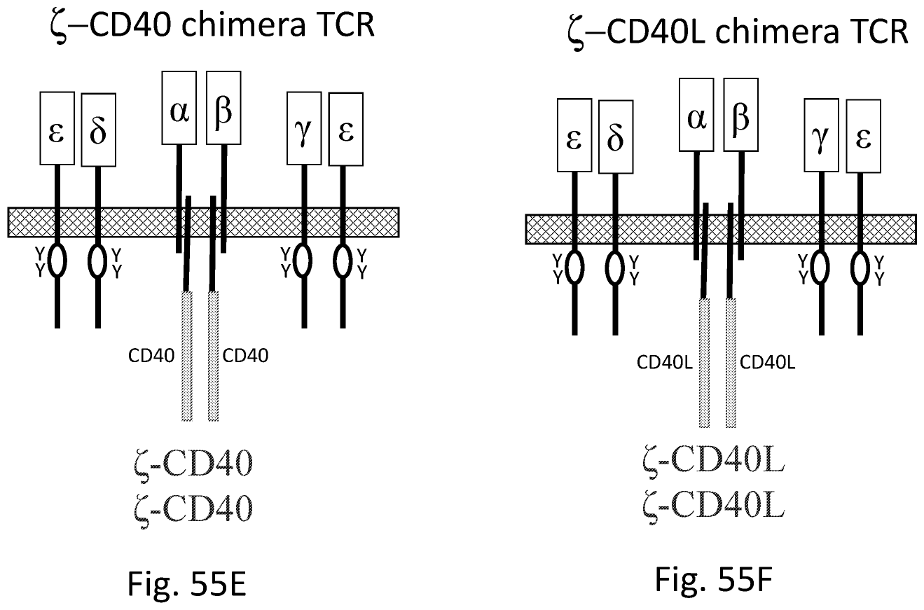

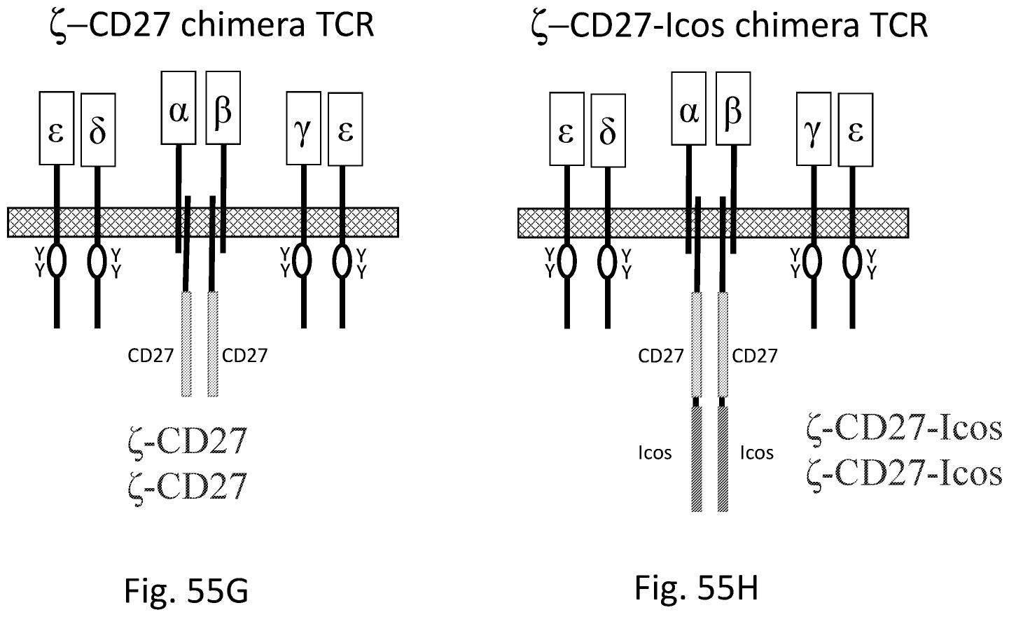

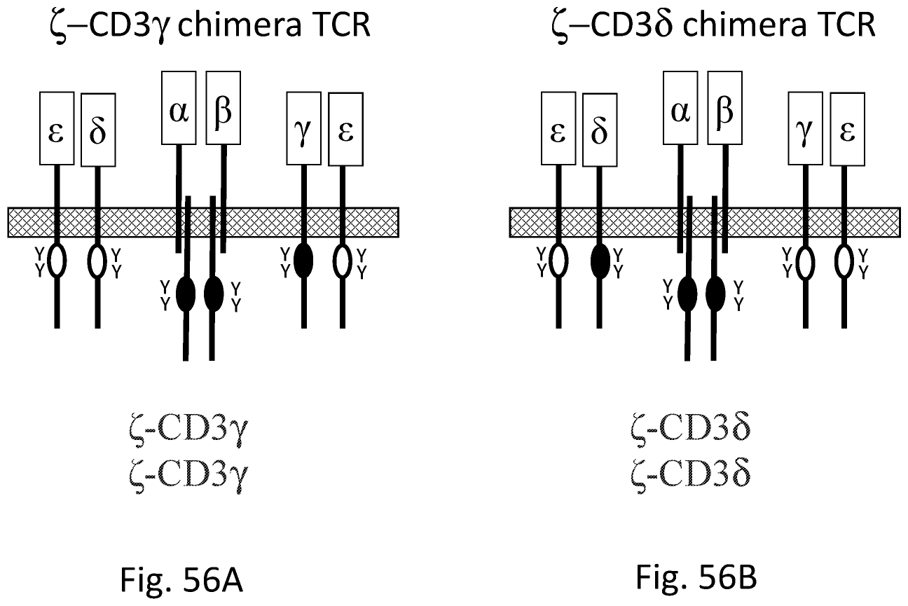

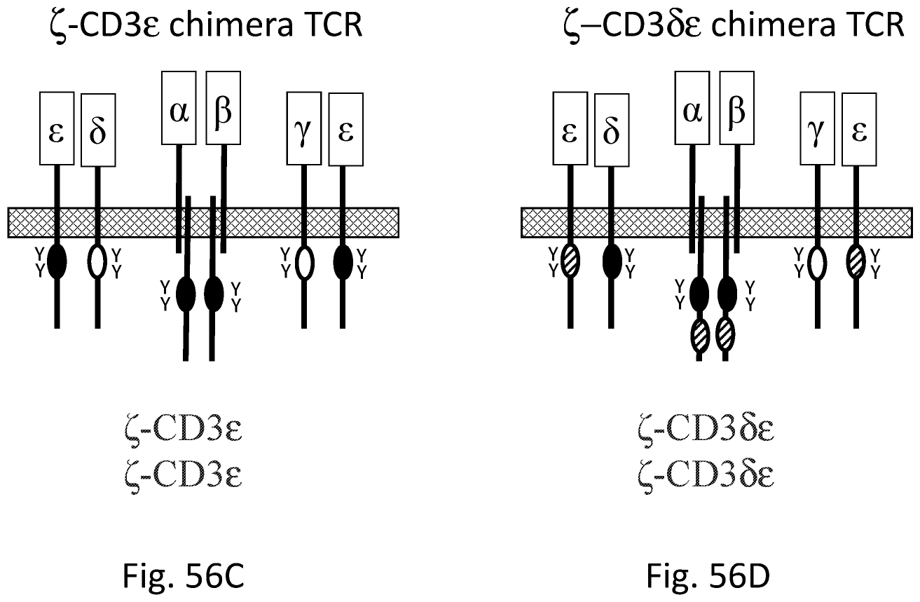

The enhanced signaling response of TCRs that contain 6F-CD3ζ to low affinity ligands raised the possibility that this property could be employed to improve T cell control of solid tumors, particularly since most tumor neo-antigens are thought to bind with much lower affinity to the TCR than pathogen-derived antigens (Stone et al., Curr. Opin. Immunol., 33: 16-22 (2015); Aleksic et al., Eur. J. Immunol., 42: 3174-3179 (2012)). OTI TCR transgenic CD8 CTL expressing 6Y-CD3ζ or 6F-CD3ζ exhibited a similar CD62L⁻ CD44⁺CD5^{hi} phenotype and expressed similar levels of surface TCR after in vitro expansion induced by high affinity N4 peptide+APC (FIGS. 41-42). However, CD8 CTL that expressed 6F-CD3ζ exhibited a lower threshold of activation when challenged with APC pulsed with low affinity antigens as shown by higher expression of the surface activation marker CD25 and the pro-inflammatory cytokine IFNγ (FIGS. 43A-43B). To test whether CTL expressing 6F-CD3ζ TCRs exhibit increased cytolytic activity against tumor cells, in vitro killing experiments were conducted using B16F10 murine melanoma cell lines engineered to express the OVA-derived agonist peptide N4 or the lower affinity APLs: A2, Y3, Q4, T4 and V4. In line with the previous results, germline 6F/6F CD3ζ OTI CTLs displayed better tumoricidal activity than 6Y/6Y CD3ζ OTI CTLs against all of the B16F10 cells that expressed lower affinity ligands, especially those that expressed the lowest affinity OTI peptides, T4 and V4 (FIGS. 44A-44H). These results were reproduced with 6Y/6Y Ert2-cre+ OTI T cells that were induced to undergo in vitro-6Y- to 6F-CD3ζ switch with 4-OH tamoxifen immediately prior to culture with melanoma cells (FIGS. 48A,B). Furthermore, WT (CD3ζ^{+/+}) OTI CTLs transduced with 6F-CD3ζ retrovirus exhibited enhanced tumoricidal activity against B16F10-V4 melanomas, indicating that suppression of endogenous WT CD3ζ is not required for the 6F-CD3ζ mediated increase in cytotoxicity (FIGS. 49 and 50). Titration of the Effector to Target (E:T) ratio demonstrated that, consistent with previous data, CTLs that expressed 6Y-CD3ζ chain exhibited slightly superior tumoricidal activity against melanomas expressing the high affinity ligand N4 compared to CTLs that expressed 6F-CD3ζ chain (FIG. 51). It was next tested if 6F-CD3ζ CTL were better able to control tumor growth in vivo using the B16F10-OVA model. Consistent with the in vitro results, 6F-CD3ζ expressing CD8 T cells were similar to or slightly less capable of controlling the growth of tumors expressing the high affinity N4 OTI TCR ligand compared to 6Y-CD3ζ T cells and did not extend the survival of B16F10-N4 tumor bearing mice (FIGS. 45A and 52A). However, 6F-CD3ζ CD8 T cells were superior to 6Y-CD3ζ in their ability to control the growth of B16F10 melanomas expressing the low affinity OTI TCR ligand, V4 (FIGS. 45B and 52B). Moreover, 6F-CD3ζ T cells extended the survival of B16F10-V4 tumor bearing mice (FIGS. 45D and 52D). Finally, it was demonstrated that the enhanced in vivo activity of 6F-CD3ζ OTI CTLs against B16F10 melanomas expressing the low affinity OTI TCR ligand V4 was cell autonomous as it was not affected by the absence of host T cells (FIGS. 46-47).

These studies are believed to be the first evidence that CD3ζ has dual functions in TCR signaling, playing a positive (amplifying) or negative (inhibitory) role in response to engagement of the TCR by high or low affinity ligands, respectively. These results also provide an explanation for previous data showing that T cells that express TCRs containing CD3ζ chains where the 3 ITAMs have been mutated or deleted can exhibit mild to severe defects in TCR signaling responses and T cell effector responses depending upon the nature (affinity) of the stimulatory interactions (Shores et al., J. Exp. Med., 185: 893-900 (1997); Pitcher et al., Eur. J. Immunol., 35: 3643-3654 (2005); Hwang et al., J. Exp. Med., 209: 1781-1795 (2012); Hwang et al., Nat. Commun., 6: 6982 (2015); Ardouin et al., Immunity, 10: 409-420 (1999)). These current and previous findings suggest several new concepts regarding TCR signaling, namely that CD3ζ may primarily serve to regulate signaling by low affinity ligands, that the ZAP-70 associated with CD3ζ ITAMs in resting T cells may predominantly provide enzyme in an inhibited rather than a poised state, and that the CD3-γ, -δ, and -ε subunits and their ITAMs are capable of eliciting an enhanced TCR signaling response to low affinity ligands in the absence of CD3ζ ITAMs. Without being bound to a particular theory or mechanism, it is proposed that the three tandem CD3ζ ITAMs can serve as sites for mono-phosphorylation in response to low affinity (short dwell time) TCR/pMHC interactions and can recruit the inhibitory tyrosine phosphatase SHP1 (FIGS. 53A-53C). The six tyrosines within the CD3ζ dimer may also reduce the probability of generating any single dual phosphorylated ITAM under conditions when the TCR is transiently engaged by ligand and ITAM tyrosine phosphorylation is limited. The present findings demonstrate that the multi-ITAM configuration of the TCR complex has evolved to perform functions that are more subtle and complex than simply signal amplification.

A limiting factor for current TCR-based cancer immunotherapy strategies is the relatively low affinity of most tumor neo-antigen-reactive TCRs (Stone et al., Curr. Opin. Immunol., 33: 16-22 (2015); Aleksic et al., Eur. J. Immunol., 42: 3174-3179, (2012)). Various next generation cell-based modifications are being explored including approaches to engineer TCRs and/or T cells to increase TCR affinity, T cell activation efficiency, tumor infiltrating potential or cytolytic potency (Morotti et al., Br. J. Cancer, 124: 1759-1776 (2021)). It is shown here that TCRs that contain 6F-CD3ζ exhibit a markedly enhanced capacity for signal transduction in response to low affinity TCR/pMHC interactions and that CTLs that express 6F-CD3ζ can better control the growth of solid tumors expressing low affinity antigens. This effect appears to occur without increasing T cell self-reactivity or predisposition to autoimmunity.

### Example 10

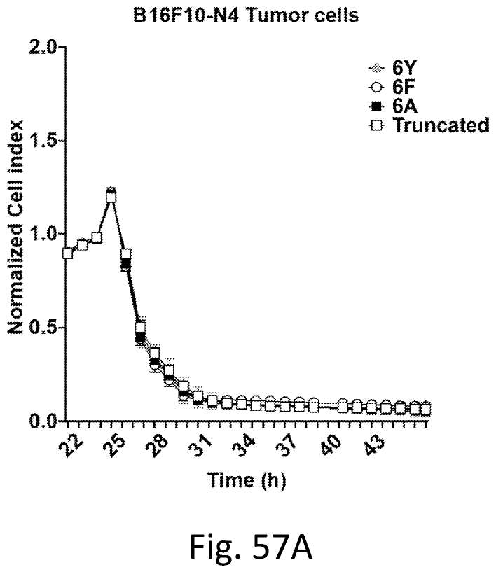

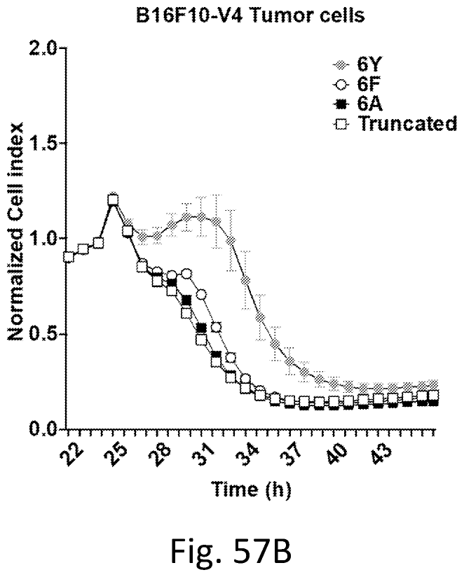

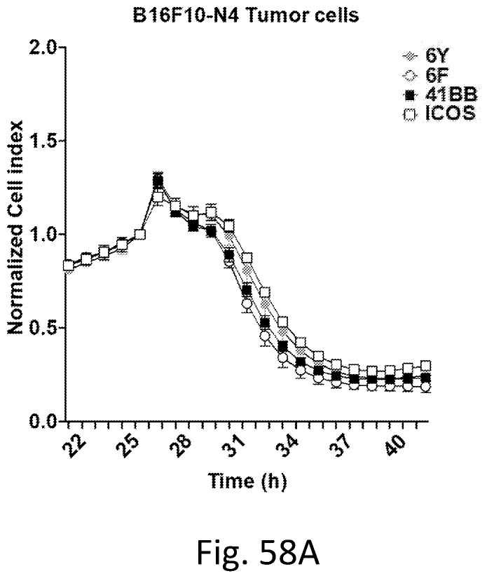

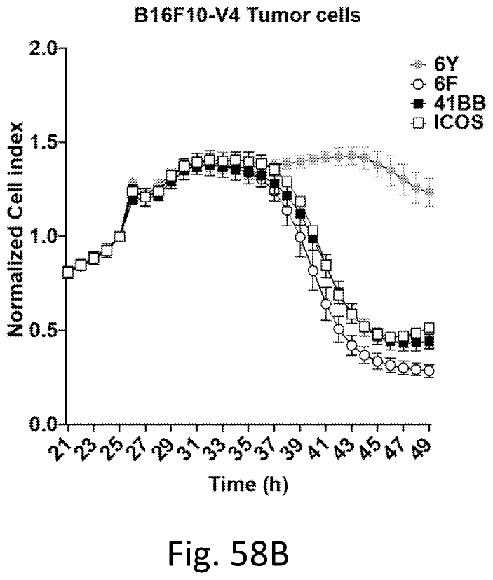

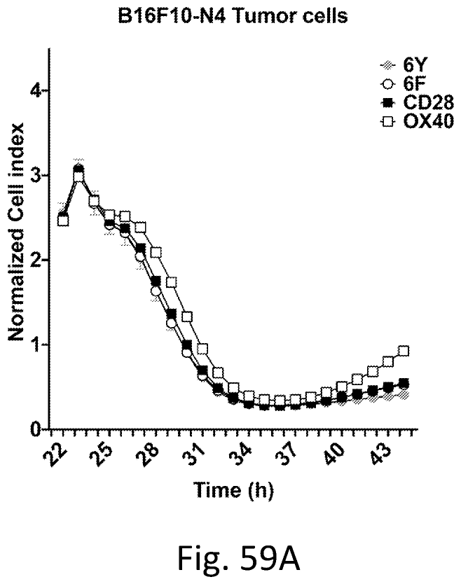

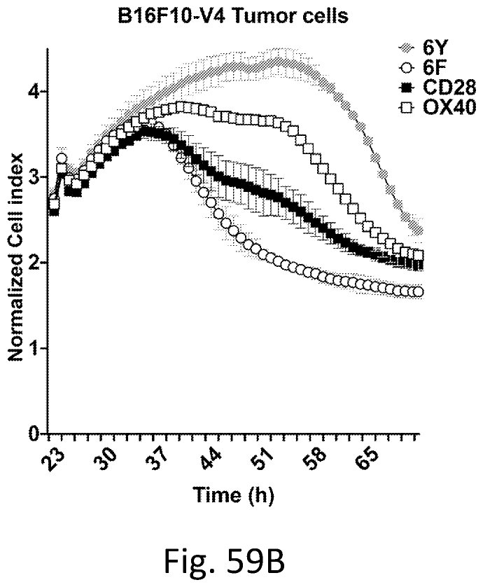

This example demonstrates that CD8 OT-1 T cells transduced with a vector expressing a CD3zeta chain in which all of the ITAM tyrosines (Y) were mutated to phenylalanine (F) provide higher in vitro killing potency toward tumor cells expressing low affinity antigen (V4) but not high affinity antigen (N4) compared to cells transduced with the vector expressing 6Y CD3zeta control (FIGS. 57A57B, 58A-58B, and 59A-59B). This example also demonstrates that CD8 OT-1 T cells transduced with a vector expressing a CD3zeta chain where all three ITAMs were deleted (truncated) or a CD3zeta chain dimer in which all of the ITAM tyrosines (Y) were mutated to Alanine (A) (FIGS. 57A-57B) provide higher in vitro killing potency toward tumor cells expressing low affinity antigens (V4) compared to cells transduced with the vector expressing 6Y CD3zeta control. This example further demonstrates that CD8 OT-1 T cells transduced with a CD3zeta chain in which all of the ITAMs were deleted and replaced by an activation motif from another (non-TCR) T cell activating receptor (e.g 4-1BB/ICOS, FIGS. 58A-58B; CD28/OX40, FIGS. 59A-59B) provide higher in vitro killing potency toward tumor cells expressing low affinity antigens (V4) compared to cells transduced with the vector expressing 6Y CD3zeta control.

In vitro T-cell mediated cytolysis of melanoma tumor cell lines by OT-1 TCR transgenic CD8⁺ T cells transduced with a vector expressing 6Y CD3zeta or one of the aforementioned modified CD3zeta chains was determined by an AGILENT XCELLIGENCE Real Time Cell Analyzer instrument (Agilent Technologies, Inc., Santa Clara, CA). B16F10 melanoma cells that express the high affinity ovalbumin peptide N4 or the low affinity ovalbumin altered peptide V4 were co-cultured with CD8⁺ T cells from OT-1 TCR transgenic mice transduced with the indicated CD3zeta construct.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A cell expressing a modified CD3 subunit chain comprising one or more of:
(a) at least one Immuno-receptor Tyrosine-based Activation Motif (ITAM) deletion;
(b) at least one exogenous intracellular hematopoietic cell signaling domain; and
(c) at least one modified ITAM comprising an amino acid sequence of Formula I:
X₁X₂X₃X₄(X₅)ₘX₆X₇X₈X₉ (Formula I),
wherein:
each of X₁ and X₆ is, independently, any amino acid residue, with the proviso that at least one of X₁ and X₆ is not tyrosine;
each of X₂, X₃, X₄, X₇, X₈ and X₉, is, independently, any amino acid residue;
each one of X₅ is, independently, any amino acid residue;
m is 6, 7, 8, 9, 10, 11, or 12; and
wherein the cell expresses an antigen-specific receptor, wherein the antigen is a cancer antigen, autoimmune disease self-antigen, or infectious disease antigen,
wherein the cell is not an immortalized cell line, and
wherein the modified CD3 subunit chain is not comprised in a chimeric antigen receptor (CAR).

2-3. (canceled)

4. The cell of claim 1, wherein the antigen-specific receptor is:
(i) a T cell receptor (TCR);
(ii) a CAR which does not comprise the modified CD3 subunit chain;
(iii) a T cell receptor Fusion Construct;
(iv) an endogenous receptor; or
(v) an exogenous receptor.

5-6. (canceled)

7. The cell of claim 1, wherein the cell is a T Cell, a regulatory T cell (Treg), a tumor infiltrating lymphocyte (TIL), a natural killer T (NKT) cell, a mucosal-associated invariant T (MAIT) cell, a gamma delta T cell (γδ T cells), or an alpha beta (αβ) T cell.

8-10. (canceled)

11. A T cell receptor (TCR) negative cell expressing one or more of:
(a) a modified CD3 subunit chain or non-CD3 subunit chain comprising at least one ITAM deletion;
(b) a modified CD3 subunit chain or non-CD3 subunit chain comprising at least one exogenous intracellular hematopoietic cell signaling domain; and
(c) a modified CD3 subunit chain or a non-CD3 modified ITAM containing subunit chain comprising at least one modified Immuno-receptor Tyrosine-based Activation Motif (ITAM) comprising an amino acid sequence of Formula I:
X₁X₂X₃X₄(X₅)ₘX₆X₇X₈X₉ (Formula I),
wherein:
each of X₁ and X₆ is, independently, any amino acid residue, with the proviso that at least one of X₁ and X₆ is not tyrosine;
each of X₂, X₃, X₄, X₇, X₈ and X₉ is, independently, any amino acid residue;
each one of X₅ is, independently, any amino acid residue; and
m is 6, 7, 8, 9, 10, 11, or 12.

12. The TCR negative cell of claim 11, wherein the TCR negative cell is:
(i) a natural killer (NK) cell;
(ii) an innate lymphoid cell (ILCs);
(iii) derived from a hematopoietic cell; or
(iv) derived from an embryonic stem cell.

13. (canceled)

14. The cell of claim 1, wherein the modified CD3 subunit chain is a CD3gamma, CD3delta, CD3epsilon chain, or CD3zeta chain.

15. (canceled)

16. The cell of claim 14, wherein the modified CD3zeta chain comprises one, two or three modified ITAMs, wherein each modified ITAM comprises an amino acid sequence of Formula I.

17. The cell of claim 1, wherein each of X₁ and X₆ is, independently, any amino acid residue except tyrosine.

18. The cell of claim 1, wherein at least one of X₁ and X₆ is, independently, selected from phenylalanine and alanine.

19. The cell of claim 1, wherein each of X₁ and X₆ is, independently, selected from phenylalanine and alanine.

20-21. (canceled)

22. The cell of claim 1, wherein the cell is derived from an induced pluripotent stem cell (iPSC), embryonic stem cell or hematopoietic stem cell.

23. The cell of claim 1, wherein the modified CD3 subunit chain or non-CD3 subunit chain comprising at least one ITAM deletion comprises an exogenous intracellular hematopoietic cell signaling domain,
optionally wherein the exogenous intracellular hematopoietic cell signaling domain comprises an intracellular T-cell signaling domain of any one of the following proteins: a 4-1BB protein, a CD27 protein, a CD28 protein, a CD8-alpha protein, a CD40 protein, a CD40L protein, an Icos protein, an OX40 protein, or any combination of the foregoing.

24. The cell of claim 1, wherein the modified CD3 subunit chain is:
(i) a CD3zeta chain comprising an ITAM deletion and an intracellular T-cell signaling domain of any one of the following proteins: a CD3gamma chain, CD3delta chain, CD3epsilon chain, or any combination of the foregoing;
(ii) a CD3gamma chain comprising an ITAM deletion and an intracellular T-cell signaling domain of any one of the following proteins: a CD3zeta chain, CD3delta chain, CD3epsilon chain, or any combination of the foregoing;
(iii) a CD3delta chain comprising an ITAM deletion and an intracellular T-cell signaling domain of any one of the following proteins: a CD3gamma chain, CD3zeta chain, CD3epsilon chain, or any combination of the foregoing;
(iv) a CD3epsilon chain comprising an ITAM deletion and an intracellular T-cell signaling domain of any one of the following proteins: a CD3gamma chain, CD3delta chain, CD3zeta chain, or any combination of the foregoing;
(v) a CD3zeta chain comprising an intracellular T-cell signaling domain of any one of the following proteins: a CD3gamma chain, CD3delta chain, CD3epsilon chain, or any combination of the foregoing;
(vi) a CD3gamma chain comprising an intracellular T-cell signaling domain of any one of the following proteins: a CD3zeta chain, CD3delta chain, CD3epsilon chain, or any combination of the foregoing;
(vii) a CD3delta chain comprising an intracellular T-cell signaling domain of any one of the following proteins: a CD3gamma chain, CD3zeta chain, CD3epsilon chain, or any combination of the foregoing; or
(viii) a CD3epsilon chain comprising an intracellular T-cell signaling domain of any one of the following proteins: a CD3gamma chain, CD3delta chain, CD3zeta chain, or any combination of the foregoing.

25. The cell of claim 1, wherein the modified CD3 subunit chain or non-CD3 subunit chain comprising at least one ITAM deletion has a truncated intracellular domain lacking any intracellular T-cell signaling domains.

26. A population of cells comprising at least one cell of claim 1.

27. A pharmaceutical composition comprising the cell of claim 1 and a pharmaceutically acceptable carrier.

28. An in vitro method of making the cell of claim 1, the method comprising modifying a cell to comprise a nucleotide sequence encoding the modified CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain.

29. The method of claim 28, wherein modifying the cell comprises introducing the nucleotide sequence encoding the modified CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain into a CD3 subunit positive cell or non-CD3 ITAM containing subunit positive cell, respectively.

30. The method of claim 28, wherein modifying the cell comprises introducing the nucleotide sequence encoding the modified CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain into a pluripotent stem cell or multipotent stem cell and the method further comprises differentiating the pluripotent stem cell or multipotent stem cell with the introduced nucleotide sequence into a cell which expresses the CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain, respectively.

31. The method of claim 28, wherein modifying the cell comprises introducing the nucleotide sequence encoding the modified CD3 subunit chain, non-CD3 subunit chain comprising at least one ITAM deletion, non-CD3 subunit chain comprising at least one exogenous intracellular hematopoietic cell signaling domain, or non-CD3 modified ITAM containing chain into the cell using transfection, transformation, transduction, electroporation, a transposon, or a genome editing technique.

32. The method of claim 31, wherein the genome editing technique uses a zinc finger nuclease, transcription activator-like effector nuclease (TALENs), a CRISPR/Cas system, or engineered meganuclease.

33. The method of claim 28, further comprising assembling the modified CD3 subunit chain with further CD3 subunit chains, assembling the non-CD3 subunit chain comprising at least one ITAM deletion with further non-CD3 ITAM containing subunit chains, assembling the non-CD3 modified ITAM containing chain with further non-CD3 ITAM containing subunit chains, assembling the non-CD3 subunit chain comprising at least one exogenous intracellular hematopoietic cell signaling domain with further non-CD3 subunit chains, or assembling the modified CD3 subunit or non-CD3 modified ITAM containing chain with further CD3 ITAM containing subunit chains.

34. The method of claim 28, further comprising suppressing expression of an endogenous, wild-type CD3 subunit chain corresponding to the modified CD3 subunit chain, suppressing expression of an endogenous, wild-type non-CD3 ITAM containing subunit chain corresponding to the non-CD3 modified ITAM containing subunit chain, suppressing expression of an endogenous, wild-type non-CD3 ITAM containing subunit chain corresponding to the non-CD3 subunit chain comprising at least one ITAM deletion, or suppressing expression of an endogenous, wild-type non-CD3 subunit chain corresponding to the non-CD3 subunit chain comprising at least one exogenous intracellular hematopoietic cell signaling domain.

35. The method of claim 34, comprising suppressing expression of the endogenous, wild-type CD3 subunit chain or endogenous wild-type non-CD3 ITAM containing chain using a zinc finger nuclease, transcription activator-like effector nuclease (TALENs), a CRISPR/Cas system, engineered meganuclease, or RNA interference.

36-40. (canceled)

41. A method of treating or preventing a condition in a subject, the method comprising:
administering a cell, or a population thereof, to the subject, in an amount effective to treat or prevent the condition in the subject,
wherein the cell expresses one or more of:
(a) a modified CD3 subunit chain or non-CD3 subunit chain comprising at least one ITAM deletion;
(b) a modified CD3 subunit chain or non-CD3 subunit chain comprising at least one exogenous intracellular hematopoietic cell signaling domain; and
(c) a modified CD3 subunit chain or non-CD3 modified ITAM containing subunit chain comprising at least one modified Immuno-receptor Tyrosine-based Activation Motif (ITAM) comprising an amino acid sequence of Formula I:
X₁X₂X₃X₄(X₅)ₘX₆X₇X₈X₉ (Formula I),
wherein:
each of X₁ and X₆ is, independently, any amino acid residue, with the proviso that at least one of X₁ and X₆ is not tyrosine;
each of X₂, X₃, X₄, X₇, X₈, and X₉ is, independently, any amino acid residue;
each one of X₅ is, independently, any amino acid residue;
m is 6, 7, 8, 9, 10, 11, or 12; and
wherein the modified CD3 subunit chain is not comprised in a chimeric antigen receptor (CAR).

42. The method of claim 41, wherein the condition is an autoimmune disease, cancer or an infectious disease.

43. A method of enhancing an antigen-specific immune response in a subject, the method comprising:
administering a cell, or a population thereof, to the subject, in an amount effective to enhance the antigen-specific immune response in the subject,
wherein the cell expresses one or more of:
(a) a modified CD3 subunit chain or non-CD3 subunit chain comprising at least one ITAM deletion;
(b) a modified CD3 subunit chain or non-CD3 subunit chain comprising at least one exogenous intracellular hematopoietic cell signaling domain; and
(c) a modified CD3 subunit chain or non-CD3 modified ITAM containing subunit chain comprising at least one modified Immuno-receptor Tyrosine-based Activation Motif (ITAM) comprising an amino acid sequence of Formula I:
X₁X₂X₃X₄(X₅)ₘX₆X₇X₈X₉ (Formula I),
wherein:
each of X₁ and X₆ is, independently, any amino acid residue, with the proviso that at least one of X₁ and X₆ is not tyrosine;
each of X₂, X₃, X₄, X₇, X₈ and X₉ is, independently, any amino acid residue;
each one of X₅ is, independently, any amino acid residue;
m is 6, 7, 8, 9, 10, 11, or 12; and
wherein the modified CD3 subunit chain is not comprised in a chimeric antigen receptor (CAR).

44. The method of claim 43, wherein the cell expresses an antigen-specific receptor.

45. The method of claim 43, wherein the antigen is a cancer antigen, autoimmune disease self-antigen, or infectious disease antigen.
